SEQUENCE LISTING (163 sequences)

SEQ ID NO: 1 - protein, 127 aa, synthetic construct
QVQLVESGGGLVKPGGSLRLSCAASGFTFSSYSMNWVRQAPGKGLEWVSYISSSSSTIYYADSVKGRFTISRDNAKNSLYLQMNSLRAEDTAVYYCARGGQYYYDSSRYGEVAFDIWGQGTMVTVSS

SEQ ID NO: 2 - protein, 9 aa, synthetic construct
FTFSSYSMN

SEQ ID NO: 3 - protein, 17 aa, synthetic construct
YISSSSSTIYYADSVKG

SEQ ID NO: 4 - protein, 20 aa, synthetic construct
ARGGQYYYDSSRYGEVAFDI

SEQ ID NO: 5 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQRRRTPPFTFGGGTKVEIK

SEQ ID NO: 6 - protein, 16 aa, synthetic construct
RSSQSLLHSNGYNYLD

SEQ ID NO: 7 - protein, 7 aa, Synthetic construct
LGSNRAS

SEQ ID NO: 8 - protein, 10 aa, Synthetic construct
MQRRRTPPFT

SEQ ID NO: 9 - protein, 457 aa, Synthetic construct
QVQLVESGGGLVKPGGSLRLSCAASGFTFSSYSMNWVRQAPGKGLEWVSYISSSSSTIYYADSVKGRFTISRDNAKNSLYLQMNSLRAEDTAVYYCARGGQYYYDSSRYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 10 - protein, 220 aa, Synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQRRRTPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 127 aa, Synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYDSSDYGEVAFDIWGQGTMVTVSS

SEQ ID NO: 12 - protein, 9 aa, Synthetic construct
FTFEAYSMN

SEQ ID NO: 13 - protein, 17 aa, Synthetic construct
YISLSGATIHYADSVKG

SEQ ID NO: 14 - protein, 20 aa, Synthetic construct
ARGGQYYYDSSDYGEVAFDI

SEQ ID NO: 15 - protein, 111 aa, Synthetic construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPWSFGGGTKVEIK

SEQ ID NO: 16 - protein, 15 aa, Synthetic Construct
RSSQSLVSNGYNYLD

SEQ ID NO: 17 - protein, 7 aa, Synthetic Construct
FGSSRAS

SEQ ID NO: 18 - protein, 9 aa, Synthetic Construct
MQRRRTPWS

SEQ ID NO: 19 - protein, 457 aa, Synthetic Construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYDSSDYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 20 - protein, 218 aa, Synthetic Construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 127 aa, Synthetic Construct
QVQLVESGGGLVKPGGSLRLSCAASGFTFGGYSMNWVRQAPGKGLEWVSYISISGATITYADSVKGRFTISRDNAKNSLYLQMNSLRAEDTAVYYCARGGQYYYDSSDYGEVAFDIWGQGTMVTVSS

SEQ ID NO: 22 - protein, 9 aa, Synthetic Construct
FTFGGYSMN

SEQ ID NO: 23 - protein, 17 aa, Synthetic Construct
YISISGATITYADSVKG

SEQ ID NO: 24 - protein, 20 aa, Synthetic Construct
ARGGQYYYDSSDYGEVAFDI

SEQ ID NO: 25 - protein, 112 aa, Synthetic Construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPPFTFGGGTKVEIK

SEQ ID NO: 26 - protein, 15 aa, Synthetic Construct
RSSQSLVSNGYNYLD

SEQ ID NO: 27 - protein, 7 aa, Synthetic Construct
FGSSRAS

SEQ ID NO: 28 - protein, 10 aa, Synthetic Construct
MQRRRTPPFT

SEQ ID NO: 29 - protein, 457 aa, Synthetic Construct
QVQLVESGGGLVKPGGSLRLSCAASGFTFGGYSMNWVRQAPGKGLEWVSYISISGATITYADSVKGRFTISRDNAKNSLYLQMNSLRAEDTAVYYCARGGQYYYDSSDYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 30 - protein, 219 aa, Synthetic Construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 127 aa, Synthetic Construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTMVTVSS

SEQ ID NO: 32 - protein, 9 aa, Synthetic Construct
FTFEAYSMN

SEQ ID NO: 33 - protein, 17 aa, Synthetic Construct
YISLSGATIHYADSVKG

SEQ ID NO: 34 - protein, 20 aa, Synthetic Construct
ARGGQYYYTSSDYGEVAFDI

SEQ ID NO: 35 - protein, 111 aa, Synthetic Construct; identical to SEQ ID NO: 15

SEQ ID NO: 36 - protein, 15 aa, Synthetic Construct
RSSQSLVSNGYNYLD

SEQ ID NO: 37 - protein, 7 aa, Synthetic Construct
FGSSRAS

SEQ ID NO: 38 - protein, 9 aa, Synthetic Construct
MQRRRTPWS

SEQ ID NO: 39 - protein, 457 aa, Synthetic Construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 40 - protein, 218 aa, Synthetic Construct; identical to SEQ ID NO: 20

SEQ ID NO: 41 - protein, 127 aa, Synthetic Construct; identical to SEQ ID NO: 31

SEQ ID NO: 42 - protein, 9 aa, Synthetic Construct
FTFEAYSMN

SEQ ID NO: 43 - protein, 17 aa, Synthetic Construct
YISLSGATIHYADSVKG

SEQ ID NO: 44 - protein, 20 aa, Synthetic Construct
ARGGQYYYTSSDYGEVAFDI

SEQ ID NO: 45 - protein, 111 aa, Synthetic Construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIK

SEQ ID NO: 46 - protein, 15 aa, Synthetic Construct
RSSQSLVSSGYNYLD

SEQ ID NO: 47 - protein, 7 aa, Synthetic Construct
FGSSRAS

SEQ ID NO: 48 - protein, 9 aa, Synthetic Construct
MQRRRTPWS

SEQ ID NO: 49 - protein, 457 aa, Synthetic Construct; identical to SEQ ID NO: 39

SEQ ID NO: 50 - protein, 218 aa, Synthetic Construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 127 aa, Synthetic Construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTLVTVSS

SEQ ID NO: 52 - protein, 9 aa, Synthetic Construct
FTFEAYSMN

SEQ ID NO: 53 - protein, 17 aa, Synthetic Construct
YISLSGATIHYADSVKG

SEQ ID NO: 54 - protein, 20 aa, Synthetic Construct
ARGGQYYYTSSDYGEVAFDI

SEQ ID NO: 55 - protein, 111 aa, Synthetic Construct
DIVLTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIK

SEQ ID NO: 56 - protein, 15 aa, Synthetic Construct
RSSQSLVSSGYNYLD

SEQ ID NO: 57 - protein, 7 aa, Synthetic Construct
FGSSRAS

SEQ ID NO: 58 - protein, 9 aa, Synthetic Construct
MQRRRTPWS

SEQ ID NO: 59 - protein, 457 aa, Synthetic Construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 60 - protein, 218 aa, Synthetic Construct
DIVLTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 127 aa, Synthetic Construct; identical to SEQ ID NO: 21

SEQ ID NO: 62 - protein, 9 aa, Synthetic Construct
FTFGGYSMN

SEQ ID NO: 63 - protein, 17 aa, Synthetic Construct
YISISGATITYADSVKG

SEQ ID NO: 64 - protein, 20 aa, Synthetic Construct
ARGGQYYYDSSDYGEVAFDI

SEQ ID NO: 65 - protein, 112 aa, Synthetic Construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPPFTFGGGTKVEIK

SEQ ID NO: 66 - protein, 15 aa, Synthetic Construct
RSSQSLVSSGYNYLD

SEQ ID NO: 67 - protein, 7 aa, Synthetic Construct
FGSSRAS

SEQ ID NO: 68 - protein, 10 aa, Synthetic Construct
MQRRRTPPFT

SEQ ID NO: 69 - protein, 457 aa, Synthetic Construct; identical to SEQ ID NO: 29

SEQ ID NO: 70 - protein, 219 aa, Synthetic Construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 119 aa, Synthetic Construct
EVQLLESGGGLVQPGGSLRLSCAASGFTFNNYWMTWVRQAPGKGLEWVSSISSSGGSIYYPDRVKGRFTISRDNSKNTLYLQMNSLRAEDTAVYYCARDERWAGAMDAWGQGTTVTVSS

SEQ ID NO: 72 - protein, 9 aa, Synthetic Construct
FTFNNYWMT

SEQ ID NO: 73 - protein, 17 aa, Synthetic Construct
SISSSGGSIYYPDRVKG

SEQ ID NO: 74 - protein, 12 aa, Synthetic Construct
ARDERWAGAMDA

SEQ ID NO: 75 - protein, 106 aa, Synthetic Construct
DIQMTQSPSSLSASVGDRVTITCKASQNINKNLDWYQQKPGKAPKLLIYETNNLQTGVPSRFSGSGSGTDFTLTISSLQPEDFATYYCYQHNSRFTFGQGTKLEIK

SEQ ID NO: 76 - protein, 11 aa, Synthetic Construct
KASQNINKNLD

SEQ ID NO: 77 - protein, 7 aa, Synthetic Construct
ETNNLQT

SEQ ID NO: 78 - protein, 8 aa, Synthetic Construct
YQHNSRFT

SEQ ID NO: 79 - protein, 449 aa, Synthetic Construct
EVQLLESGGGLVQPGGSLRLSCAASGFTFNNYWMTWVRQAPGKGLEWVSSISSSGGSIYYPDRVKGRFTISRDNSKNTLYLQMNSLRAEDTAVYYCARDERWAGAMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 80 - protein, 213 aa, Synthetic Construct
DIQMTQSPSSLSASVGDRVTITCKASQNINKNLDWYQQKPGKAPKLLIYETNNLQTGVPSRFSGSGSGTDFTLTISSLQPEDFATYYCYQHNSRFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 119 aa, Synthetic Construct
EVQLVESGGGLVQPGGSLRLSCAASGFTFTNFWMAWIRQAPGKGLEWVASISSSGGSIYYPDSVKDRFTISRDNSKNTLYLQMNSLRAEDTAVYYCVKFHHYSGGGDAWGQGTLVTVSS

SEQ ID NO: 82 - protein, 9 aa, Synthetic Construct
FTFTNFWMA

SEQ ID NO: 83 - protein, 17 aa, Synthetic Construct
SISSSGGSIYYPDSVKD

SEQ ID NO: 84 - protein, 12 aa, Synthetic Construct
VKFHHYSGGGDA

SEQ ID NO: 85 - protein, 106 aa, Synthetic Construct
DIQMTQSPSSLSASVGDRVTITCKASQNINKYLDWYQQKPGKAPKLLIHYTNNLHTGIPSRFSGSGSGTDYTLTISSLQPEDFATYYCLQHSSRWTFGGGTKVEIK

SEQ ID NO: 86 - protein, 11 aa, Synthetic Construct
KASQNINKYLD

SEQ ID NO: 87 - protein, 7 aa, Synthetic Construct
YTNNLHT

SEQ ID NO: 88 - protein, 8 aa, Synthetic Construct
LQHSSRWT

SEQ ID NO: 89 - protein, 449 aa, Synthetic Construct
EVQLVESGGGLVQPGGSLRLSCAASGFTFTNFWMAWIRQAPGKGLEWVASISSSGGSIYYPDSVKDRFTISRDNSKNTLYLQMNSLRAEDTAVYYCVKFHHYSGGGDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 90 - protein, 213 aa, Synthetic Construct
DIQMTQSPSSLSASVGDRVTITCKASQNINKYLDWYQQKPGKAPKLLIHYTNNLHTGIPSRFSGSGSGTDYTLTISSLQPEDFATYYCLQHSSRWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 119 aa, Synthetic Construct
EVQLVESGGGLVQPGGSLRLSCAASGFTFNNYWMTWVRQAPGKGLEWVSSISSSGGSIYYPDSVKDRFTISRDNAKNSLYLQMNSLRAEDMAVYYCARLYYYDGGGDAWGQGTLVTVSS

SEQ ID NO: 92 - protein, 9 aa, Synthetic Construct
FTFNNYWMT

SEQ ID NO: 93 - protein, 17 aa, Synthetic Construct
SISSSGGSIYYPDSVKD

SEQ ID NO: 94 - protein, 12 aa, Synthetic Construct
ARLYYYDGGGDA

SEQ ID NO: 95 - protein, 106 aa, Synthetic Construct
GIQMTQSPSSLSASVGDRVTITCKASQDINKYLDWYQQKPGKAPKLLIYNTNNLHTGIPSRFSGSGSGTDYTLTISSLQPEDFATYYCLQHISRWTFGGGTKVEIK

SEQ ID NO: 96 - protein, 11 aa, Synthetic Construct
KASQDINKYLD

SEQ ID NO: 97 - protein, 7 aa, Synthetic Construct
NTNNLHT

SEQ ID NO: 98 - protein, 8 aa, Synthetic Construct
LQHISRWT

SEQ ID NO: 99 - protein, 449 aa, Synthetic Construct
EVQLVESGGGLVQPGGSLRLSCAASGFTFNNYWMTWVRQAPGKGLEWVSSISSSGGSIYYPDSVKDRFTISRDNAKNSLYLQMNSLRAEDMAVYYCARLYYYDGGGDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 100 - protein, 213 aa, Synthetic Construct
GIQMTQSPSSLSASVGDRVTITCKASQDINKYLDWYQQKPGKAPKLLIYNTNNLHTGIPSRFSGSGSGTDYTLTISSLQPEDFATYYCLQHISRWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 107 aa, Synthetic Construct [sequence omitted]

SEQ ID NO: 102 - protein, 330 aa, Synthetic Construct [sequence omitted]

SEQ ID NO: 103 - protein, 330 aa, Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 330 aa, Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 106 - protein, 330 aa, Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 107 - protein, 1211 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 108 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 109 - protein, 1192 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 110 - protein, 1258 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 111 - protein, 1240 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 112 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 113 - protein, 552 aa, Homo sapiens
QSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSK

SEQ ID NO: 114 - protein, 21 aa, Homo sapiens
TEKDCLNLDKNLIKYDLQNLK

SEQ ID NO: 115 - protein, 31 aa, Homo sapiens
CYIKETEKDCLNLDKNLIKYDLQNLKPYTKY

SEQ ID NO: 116 - protein, 29 aa, Homo sapiens
RPPRDRNGPHERYHLEVEAGNTLVRNESH

SEQ ID NO: 117 - protein, 31 aa, Homo sapiens
CRPPRDRNGPHERYHLEVEAGNTLVRNESHK

SEQ ID NO: 118 - protein, 17 aa, Homo sapiens
RNGPHERYHLEVEAGNT

SEQ ID NO: 119 - protein, 9 aa, synthetic construct
  VARIANT 4: note = X is S, E, or G
  VARIANT 5: note = X is S, A, or G
FTFXXYSMN

SEQ ID NO: 120 - protein, 18 aa, synthetic construct
  VARIANT 4: note = X is S, L, or I
  VARIANT 6: note = X is S or G
  VARIANT 7: note = X is S or A
  VARIANT 10: note = X is Y, H, or T
YISXSXXTIXYYADSVKG

SEQ ID NO: 121 - protein, 20 aa, synthetic construct
  VARIANT 9: note = X is D or T
  VARIANT 12: note = X is R or D
ARGGQYYYXSSXYGEVAFDI

SEQ ID NO: 122 - protein, 16 aa, synthetic construct
  VARIANT 8: note = X is H or absent
RSSQSLLXSNGYNYLD

SEQ ID NO: 123 - protein, 7 aa, synthetic construct
  VARIANT 1: note = X is L or F
  VARIANT 4: note = X is N or S
XGSXRAS

SEQ ID NO: 124 - protein, 10 aa, synthetic construct
  VARIANT 8: note = X is P or W
  VARIANT 9: note = X is F or S
  VARIANT 10: note = X is T or F
MQRRRTPXXX

SEQ ID NO: 125 - DNA, 381 bp, synthetic construct
  source 1..381: mol_type = other DNA
caggtgcagctggtggagtctgggggaggcttggtcaagcctggagggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagctgaggacacggcggtgtactactgcgccagaggtggacaatactactacgacagcagcagatacggtgaggtagcattcgacatatggggtcagggtacaatggtcaccgtctcctca

SEQ ID NO: 126 - DNA, 339 bp, synthetic construct
  source 1..339: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcagagaagacgcactcctcctttcacttttggcggagggaccaaggttgagatcaaa

SEQ ID NO: 127 - DNA, 381 bp, synthetic construct
  source 1..381: mol_type = other DNA
gaagtgcagcttgtggagtccggtggcggactggtccagccgggcggatctctgagactttcgtgtgccgcctcgggattcaccttcgaagcgtattccatgaactgggtcagacaggcccccggaaagggcctggaatgggtgtcgtacattagcctgtcgggggccaccatccattacgccgatagcgtgaagggccggttcacaatctcccgggacaacgccaagaactccctctacctccaaatgaacagcctgcgcgctgaggacactgctgtgtactattgcgcgaggggtggccagtactactacgactcaagcgactacggcgaagtggcattcgatatctggggacaggggaccatggtcaccgtcagctcc

SEQ ID NO: 128 - DNA, 333 bp, synthetic construct
  source 1..333: mol_type = other DNA
gatatcgtgatgacacagtcccctctgtccctccctgtgacccccggagaaccagcctctatttcctgccggtcctcccaatccctggtgtccaacggttataactacctggattggtacttgcaaaagcccggacagagcccccagctgctcatctacttcggaagctcacgcgcgagcggggtgccggataggttttcgggatccggaagcggcaccgacttcacgctgaagatctcgagagtcgaggccgaggacgtgggcgtgtactactgtatgcagcggcggcgcaccccctggtccttcggcggcggaactaaggtcgagatcaag

SEQ ID NO: 129 - DNA, 381 bp, synthetic construct
  source 1..381: mol_type = other DNA
caagtgcagcttgtggagtccggtggcggactggtcaagccgggcggatctctgagactttcgtgtgccgcctcgggattcaccttcggcggatattccatgaactgggtcagacaggcccccggaaagggcctggaatgggtgtcgtacattagcatctcgggggccaccatcacttacgccgatagcgtgaagggccggttcacaatctcccgggacaacgccaagaactccctctacctccaaatgaacagcctgcgcgctgaggacactgctgtgtactattgcgcgaggggtggccagtactactacgactcaagcgactacggcgaagtggcattcgatatctggggacaggggaccatggtcaccgtcagctcc

SEQ ID NO: 130 - DNA, 336 bp, synthetic construct
  source 1..336: mol_type = other DNA
gatatcgtgatgacacagtcccctctgtccctccctgtgacccccggagaaccagcctctatttcctgccggtcctcccaatccctggtgtccaacggttataactacctggattggtacttgcaaaagcccggacagagcccccagctgctcatctacttcggaagctcacgcgcgagcggggtgccggataggttttcgggatccggaagcggcaccgacttcacgctgaagatctcgagagtcgaggccgaggacgtgggcgtgtactactgtatgcagcggcggcgcaccccgcccttcaccttcggcggcggaactaaggtcgagatcaag

SEQ ID NO: 131 - DNA, 381 bp, synthetic construct
  source 1..381: mol_type = other DNA
gaagtgcagcttgtggagtccggtggcggactggtccagccgggcggatctctgagactttcgtgtgccgcctcgggattcaccttcgaagcgtattccatgaactgggtcagacaggcccccggaaagggcctggaatgggtgtcgtacattagcctgtcgggggccaccatccattacgccgatagcgtgaagggccggttcacaatctcccgggacaacgccaagaactccctctacctccaaatgaacagcctgcgcgctgaggacactgctgtgtactattgcgcgaggggtggccagtactactacacctcaagcgactacggcgaagtggcattcgatatctggggacaggggaccatggtcaccgtcagctcc

SEQ ID NO: 132 - DNA, 333 bp, synthetic construct; identical to SEQ ID NO: 128
  source 1..333: mol_type = other DNA

SEQ ID NO: 133 - DNA, 381 bp, synthetic construct; identical to SEQ ID NO: 131
  source 1..381: mol_type = other DNA

SEQ ID NO: 134 - DNA, 333 bp, synthetic construct
  source 1..333: mol_type = other DNA
gatatcgtgatgacacagtcccctctgtccctccctgtgacccccggagaaccagcctctatttcctgccggtcctcccaatccctggtgtcctcgggttataactacctggattggtacttgcaaaagcccggacagagcccccagctgctcatctacttcggaagctcacgcgcgagcggggtgccggataggttttcgggatccggaagcggcaccgacttcacgctgaagatctcgagagtcgaggccgaggacgtgggcgtgtactactgtatgcagcggcggcgcaccccctggtccttcggcggcggaactaaggtcgagatcaag

SEQ ID NO: 135 - DNA, 381 bp, synthetic construct
  source 1..381: mol_type = other DNA
gaggtgcagctggtcgaaagcggaggagggctggtgcagcctggaggatccctgcggctctcatgtgccgcctccggctttaccttcgaagcctactccatgaactgggtcagacaggctcccgggaagggactggaatgggtcagctacatttcgctgtccggagccaccatccactacgctgactcagttaagggacgcttcaccatctcccgggataatgcaaagaactccctgtacctccaaatgaattcactgagggccgaggacactgccgtgtactactgcgcccggggaggtcaatactattacacctcctccgactacggcgaagtggccttcgatatctggggccaaggaaccctcgtgactgtctcctcc

SEQ ID NO: 136 - DNA, 333 bp, synthetic construct
  source 1..333: mol_type = other DNA
gacatcgtgctgacccagtcaccgctttccttgcccgtgactcctggggaaccggcctccatttcgtgccggtccagccagtccctggtgtcctccggctacaattacctggattggtacctccaaaagcccggacagtccccacaactgctcatctacttcgggagctcaagggcctcaggagtgccggatcgcttctcgggttccggaagcgggactgacttcactctgaaaatcagccgcgtggaagcagaggacgtgggcgtgtactactgcatgcagcgcaggagaaccccctggtcctttggcggtggaacgaaggtcgaaatcaag

SEQ ID NO: 137 - DNA, 381 bp, synthetic construct; identical to SEQ ID NO: 129
  source 1..381: mol_type = other DNA

SEQ ID NO: 138 - DNA, 336 bp, synthetic construct
  source 1..336: mol_type = other DNA
gatatcgtgatgacacagtcccctctgtccctccctgtgacccccggagaaccagcctctatttcctgccggtcctcccaatccctggtgtcctccggttataactacctggattggtacttgcaaaagcccggacagagcccccagctgctcatctacttcggaagctcacgcgcgagcggggtgccggataggttttcgggatccggaagcggcaccgacttcacgctgaagatctcgagagtcgaggccgaggacgtgggcgtgtactactgtatgcagcggcggcgcaccccgcccttcaccttcggcggcggaactaaggtcgagatcaag

SEQ ID NO: 139 - DNA, 357 bp, synthetic construct
  source 1..357: mol_type = other DNA
gaagtgcagcttctggagtccggtggcggactggtccagccgggcggatctctgagactttcgtgtgccgcctcgggattcaccttcaacaactattggatgacctgggtcagacaggcccccggaaagggcctggaatgggtgtcgtcaattagctcctcggggggatccatctactaccctgatcgcgtgaagggccggttcacaatctcccgggacaacagcaagaacaccctctacctccaaatgaacagcctgcgcgctgaggacactgctgtgtactattgcgcgagggacgagagatgggccggcgcaatggatgcctggggacaggggaccaccgtcaccgtcagctcc

SEQ ID NO: 140 - DNA, 318 bp, synthetic construct
  source 1..318: mol_type = other DNA
gatattcagatgacccagtccccatcatccctgtccgcctccgtgggcgaccgcgtgacgatcacttgcaaagccagccagaatatcaacaagaacctggattggtaccaacagaagccggggaaggcccctaagctgctgatctacgaaaccaacaacttgcaaactggcgtgccgtcaaggttcagcggttccgggtcgggcaccgacttcaccctgaccatttcctcgctgcaacccgaggacttcgcgacctactactgctatcagcacaacagccggttcaccttcggacagggcaccaagctcgagatcaag

SEQ ID NO: 141 - DNA, 357 bp, synthetic construct
  source 1..357: mol_type = other DNA
gaagtgcagctcgtggagtcgggtggaggccttgtgcaaccgggaggatccctgcggctctcctgcgccgcatcaggcttcacgttcaccaacttttggatggcctggattagacaggcaccggggaagggactggaatgggtggcgtccattagctcgtccggaggatccatctactatcctgactcagtgaaggacaggtttaccatctcccgggacaacagcaagaacactctgtacctccaaatgaactcgctgcgcgccgaggacaccgccgtgtactactgcgtgaagttccatcactactccggcggaggagatgcctggggacagggtactctcgtgactgtgtcgtcc

SEQ ID NO: 142 - DNA, 318 bp, synthetic construct
  source 1..318: mol_type = other DNA
gacatccagatgacccagagcccctcctccctgtccgcgtctgtgggcgaccgcgtgaccattacgtgcaaagcttcccagaacattaacaagtacctggattggtaccagcagaagcctggaaaggcccccaagctgttgatccactacacaaacaacctccacactggtatcccgtcccggttctcggggtccggatcgggaactgactacaccctgaccatcagcagcctgcagcctgaagatttcgccacctattactgcctgcaacactcctcgcgctggaccttcggcgggggtactaaggtcgagatcaag

SEQ ID NO: 143 - DNA, 357 bp, synthetic construct
  source 1..357: mol_type = other DNA
gaagtgcagctcgtggagtcgggtggaggccttgtgcaaccgggaggatccctgcggctctcctgcgccgcatcaggcttcacgttcaacaactactggatgacttgggtcagacaggcaccggggaagggactggaatgggtgtccagcattagctcgtccggaggatccatctactatccggactcagtgaaggacaggtttaccatctcccgggacaacgcaaagaactccctgtacctccaaatgaactcgctgcgcgccgaggacatggccgtgtactactgcgcgaggctgtactactacgatggggggggcgatgcctggggacagggaaccctagtgactgtgtcgtcc

SEQ ID NO: 144 - DNA, 318 bp, synthetic construct
  source 1..318: mol_type = other DNA
ggaatccagatgacacagagcccgtctagcctgtcagcatccgtgggggacagggtcaccatcacctgtaaagccagccaggatattaacaagtacctggactggtaccagcagaagcccgggaaggccccgaagctcctgatctacaacaccaacaacttgcacaccggaattccgtcccgcttttcgggatcgggatccgggaccgattacaccctgactatctcctccctgcaacccgaggacttcgccacttactattgcctccaacacatttcccggtggactttcggcggcggcaccaaggtcgagatcaag

SEQ ID NO: 145 - protein, 1310 aa, Macaca fascicularis
MTMCLWLKLLAFVFAFLDTEVFVTGQGSTLSPTGRRTTKMPSVPLSSDPLPTHTTAFSPASISERENDFSETTPSLSSDNTSTQVSPDSLDNASAFNTTGVSSALTPHLPTHADSQTPSTGTDTQTPSGSAANTTLSPTPRSNDISDVPGERSTASTFPTDPISPLATTLIPARNSSAALPARTSNTTITANTSVSYLNASETTTPSPSGSTVISTPTIATTTSKPTCAEKYATIPVDYLYNNKTKLFTAKLNVNENVECTNNNHTHNICTNNEVLNLPECKEMNVFVSHNSCTDRHKELKLDVPPEVEKFQLDDCTPDVEANTTICLKWKIIETFACDKSKITYRFQCGNKTYNKEGIYLENLEPEYEYKCDSEILYNNHKYINITKLIKTDFGIPGQPQNVVCRHEDAHQGVITWNPPQRSFHNFTLCYVNKPAKKCLILDKHLTTYHLQNLKPYTNYSLSLHAYIIAKVQRNGTAATCNFTTESAPPSQVQNMIVSTSDNSMHVKCEVPRDVNGPTGLYHLEVEAGNTLVRNLSQSKCDFSVNNLQYSTYYNLKAYYHNGKYSGEPVILRESTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDIVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKNKNRNSNVIPYDYNRVPLKHELEMSKESDHDSDESSDDDSDSEEPSKYINASFIMSYWKPEVMIAAQGPLKETIGDFWQMIFQRKVKVIVMLTELKHGDQEICAQYWGEGKQTYGDIEVDMKDTNKSSTYTLRVFELRHSKRKDSRTVYQYQYTNWSVEQLPAEPKELVSLIQVLKEKLPQKNFSEGNKHHKSTPLLIHCRDGSQQTGIFCALLNLLESAETEEVVDIFQVVKALRKARPGMVSTFEQYQFLYDIIASTYPAQNGQVKKNNHQEDKIEFDNEVDKVKQDANCVNPLGATEKLPEAKEQATGSEPTSGTEGPEHSVNGPASPALNQGS

SEQ ID NO: 146 - protein, 1310 aa, Macaca mullata
MTMCLWLKLLAFVFAFLDTEVFVTGQGSTLSPTGRRTTKMPSVPLSSDPLPTHTTAFSPASISERENDFSETTPSLSSDNTSTHVSPDSLDNASAFNTTGVSSALTPHLPTHADSQTPSTGTDTQTPSGSAANTTLSPTPRSNDISDVPGERSTASTFPTDPISPLATTLIPARNSSAALPARTSNTTITANTSVSYLNASETTTPSPSGSTVISTPTIATTTSKPTCAEKYATIPVDYLYNNKTKLFTAKLNVNENVECTNNNHTHNICTNNEVLNLPECKEMNVFVSHNSCTDRHKELKLDVPPEVEKFQLDDCTPDVEANTTICLKWKIIETFACDKSKITYRFQCGNKTYNKEGIYLENLEPEYEYKCDSEILYNNHKYINITKLIKTDFGIPGQPQNVVCRHEDAHQGVITWNPPQRSFHNFTLCYVSKTAKKCLSLDKHLTTYHLQNLKPYTNYSLSLHAYIIAKVQRNGTAATCNFTTESAPPSQVQNMIVSTSDNSMRVKCEAPRDVNGPTELYLLEVEAGNTLVRNLSQSECDFSVNNLQYSTYYNLKAYYHNGKYSGEPVILRESTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKNKNRNSNVIPYDYNRVPLKHELEMSKESDHDSDESSDDDSDSEEPSKYINASFIMSYWKPEVMIAAQGPLKETIGDFWQMIFQRKVKVIVMLTELKHGDQEICAQYWGEGKQTYGDIEVDMKDTNKSSTYTLRVFELRHSKRKDSRTVYQYQYTNWSVEQLPAEPKELVSLIQVLKEKLPQKNSSEGNKHHKSTPLLIHCRDGSQQTGIFCALLNLLESAETEEVVDIFQVVKALRKARPGMVSTFEQYQFLYDIIASTYPAQNGQVKKNNHQEDKIEFDNEVDKVKQDANCVNPLGATEKLPEAKEQATGSEPTSGTEGPEHSVNGPASPALNQGS

SEQ ID NO: 147 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 148 - protein, 293 aa, Shigella dysenteriae
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 149 - protein, 297 aa, Escherichia coli
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 150 - DNA, 381 bp, synthetic construct
  source 1..381: mol_type = other DNA
gaggtgcagctggtggagtctgggggaggcttggtacagcctggggggtccctgagactctcctgtgcagcctctggattcaccttcgaagcatatagcatgaactgggtccgccaggctccagggaaggggctggagtgggtttcatacattagtctcagtggtgccaccatacactacgcagactctgtgaagggccgattcaccatctccagggacaacgccaagaactcactgtatctgcaaatgaacagcctgagagccgaggacacggcggtgtattactgcgccagaggtggacaatactactacgacagcagtgattacggtgaggtagcattcgacatatggggtcagggtacaatggtcaccgtctcctca

SEQ ID NO: 151 - DNA, 333 bp, synthetic construct
  source 1..333: mol_type = other DNA
gatattgtgatgactcagtctccactctccctgcccgtcacccctggagagccggcctccatctcctgcaggtctagtcagagcctggtcagtaatggatacaactatttggattggtacctgcagaagccagggcagtctccacagctcctgatctatttcggttcttcccgggcctccggggtccctgacaggttcagtggcagtggatcaggcacagattttacactgaaaatcagcagagtggaggctgaggatgttggggtttattactgcatgcagagaagacgcactccttggtcttttggcggagggaccaaggttgagatcaaa

SEQ ID NO: 152 - DNA, 381 bp, synthetic construct
  source 1..381: mol_type = other DNA
caggtgcagctggtggagtctgggggaggcttggtcaagcctggagggtccctgagactctcctgtgcagcctctggattcaccttcggaggatatagcatgaactgggtccgccaggctccagggaaggggctggagtgggtttcatacattagtatcagtggtgccaccataacctacgcagactctgtgaagggccgattcaccatctccagggacaacgccaagaactcactgtatctgcaaatgaacagcctgagagccgaggacacggcggtgtactactgcgccagaggtggacaatactactacgacagcagcgattatggtgaggtagcattcgacatatggggtcagggtacaatggtcaccgtctcctca

SEQ ID NO: 153 - DNA, 336 bp, synthetic construct
  source 1..336: mol_type = other DNA
gatattgtgatgactcagtctccactctccctgcccgtcacccctggagagccggcctccatctcctgcaggtctagtcagagcctggtcagtaatggatacaactatttggattggtacctgcagaagccagggcagtctccacagctcctgatctatttcggttcttcccgggcctccggggtccctgacaggttcagtggcagtggatcaggcacagattttacactgaaaatcagcagagtggaggctgaggatgttggggtttattactgcatgcagagaagacgcactcctcctttcacttttggcggagggaccaaggttgagatcaaa

SEQ ID NO: 154 - DNA, 381 bp, synthetic construct
  source 1..381: mol_type = other DNA
gaggtgcagctggtggagtctgggggaggcttggtacagcctggggggtccctgagactctcctgtgcagcctctggattcaccttcgaagcatatagcatgaactgggtccgccaggctccagggaaggggctggagtgggtttcatacattagtctcagtggtgccaccatacactacgcagactctgtgaagggccgattcaccatctccagggacaacgccaagaactcactgtatctgcaaatgaacagcctgagagccgaggacacggcggtgtattactgcgccagaggtggacaatactactacacgagcagtgattacggtgaggtagcattcgacatatggggtcagggtacaatggtcaccgtctcctca

SEQ ID NO: 155 - DNA, 333 bp, synthetic construct; identical to SEQ ID NO: 151
  source 1..333: mol_type = other DNA

SEQ ID NO: 156 - DNA, 357 bp, synthetic construct
  source 1..357: mol_type = other DNA
gaggtgcagctgttggagtctgggggaggcttggtacagcctggcgggtccctgagactctcctgtgcagcctctggattcacctttaataattattggatgacatgggtccgccaggctccagggaaggggctggagtgggtctcatctattagttccagtggtggtagcatttactaccccgacagggtgaagggccggttcaccatctccagagacaattccaagaacacgctgtatctgcaaatgaacagcctgagagccgaggacacggcggtgtactactgcgcaagagacgagagatgggcaggtgctatggatgcctgggggcaagggaccacggtcaccgtctcctca

SEQ ID NO: 157 - DNA, 318 bp, synthetic construct
  source 1..318: mol_type = other DNA
gacatccagatgacccagtctccatcctccctgtctgcatctgtaggagacagagtcaccatcacttgcaaggcaagtcagaatattaacaagaatttagactggtatcagcagaaaccagggaaagcccctaagctcctgatctatgagacgaataacttgcaaacaggggtcccatcaaggttcagtggcagtggatctgggacagatttcactctcaccatcagcagtctgcaacctgaagattttgcaacttactactgttatcagcataattctagatttacttttggccaggggaccaagctggagatcaaa

SEQ ID NO: 158 - DNA, 357 bp, synthetic construct
  source 1..357: mol_type = other DNA
gaggtgcagctggtggagtctgggggaggcttggtacagcctggagggtccctgagactctcctgtgcagcctctggattcacctttaccaatttttggatggcgtggatccgccaggctccagggaaggggctggagtgggtcgcaagtattagttcaagtggtggtagcatctactaccctgactccgtgaaggaccggttcaccatctccagagacaattccaagaacacgctgtatctgcaaatgaacagcctgagagccgaggacacggcggtgtactactgcgtcaagtttcaccactattcaggcggcggcgatgcttggggccaagggaccctggtcaccgtctcctca

SEQ ID NO: 159 - DNA, 318 bp, synthetic construct
  source 1..318: mol_type = other DNA
gacatccagatgacccagtctccatcctccctgtctgcatctgtaggagacagagtcaccatcacttgcaaagcaagtcagaatattaacaagtatttagattggtatcagcagaaaccagggaaagcccctaagctcctgatccattacactaacaacttgcacaccgggattccatcaaggttcagtggcagtggatctgggacagattatactctcaccatcagcagtctgcaacctgaagattttgcaacttactactgtctgcagcacagttccagatggacattcggcggagggaccaaggtggagatcaaa

SEQ ID NO: 160 - DNA, 357 bp, synthetic construct
  source 1..357: mol_type = other DNA
gaggtgcagctggtggagtctgggggaggcttggtacagcctggagggtccctgagactctcctgtgcagcctctggattcaccttcaataactattggatgacgtgggtccgccaggctccagggaaggggctggagtgggtttcatccattagtagtagtggcggtagtatatactaccctgactctgtgaaggatcgattcaccatctccagagacaatgccaagaactcactgtatctgcaaatgaacagcctgagagctgaggacatggcggtgtactactgcgccaggttgtactactacgacgggggaggggatgcgtggggccaaggaaccctggtcaccgtctcctca

SEQ ID NO: 161 - DNA, 318 bp, synthetic construct
  source 1..318: mol_type = other DNA
ggcatccagatgacccagtctccatcctccctgtctgcatctgtaggagacagagtcaccatcacttgcaaggcgagtcaggacattaataagtatttagattggtatcagcagaaaccagggaaagcccctaagctcctgatctacaatacaaacaatttgcatacagggatcccatcaaggttcagtggaagtggatctgggacagattatactcttaccatcagcagcctgcagcctgaagattttgcaacatattactgtcttcaacacatatctagatggacgttcggcggagggaccaaggtggagatcaaa

SEQ ID NO: 162 - DNA, 1473 bp, Synthetic construct
  source 1..1473: mol_type = other DNA
ggtaccgccaccatggatctgctgcacaagaacatgaagcacctgtggttctttctgctgctggtggccgctcctagatgggtgctgtctgaagtgcagctggtggaatctggcggaggattggttcagcctggcggctctctgagactgtcttgtgctgcctctggcttcaccttcgaggcctactccatgaactgggtccgacaggctcctggcaaaggactggaatgggtgtcctacatctctctgtctggcgccaccatccactacgccgattctgtgaagggcagattcaccatcagccgggacaacgccaagaactccctgtacctgcagatgaacagcctgagagccgaggacaccgccgtgtactattgtgctagaggcggccagtactactacacctcctctgattacggcgaggtggccttcgatatctggggccagggaacaatggtcaccgtgtcctctgcttccaccaagggaccttccgtgtttcctctggctccttccagcaagtctacctctggtggaaccgctgctctgggctgcctggtcaaggattactttcctgagcctgtgaccgtgtcttggaactctggtgctctgacctccggcgtgcacacatttccagctgtgctgcagtcctccggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcggaccctctgtgttcctgtttccacctaagcctaaggacaccctgatgatctctcggacccctgaagtgacctgcgtggtcgtgtgtgtgtctcacgaggacccagaagtgaagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaggtttacacactgcctccatctcgggacgagctgaccaagaatcaggtgtccctgacctgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaacgcctacacacagaagtccctgtctctgtcccctggcaagtgatagaagctt

SEQ ID NO: 163 - DNA, 756 bp, synthetic construct
  source 1..756: mol_type = other DNA
ggatccgccaccatggatctgctgcacaagaacatgaagcacctgtggttctttctgctgctggtggccgctcctagatgggtgctgtctgacatcgtgatgacccagtctccactgagcctgcctgttacacctggcgagcctgccagcatctcctgcagatcttcccagtctctggtgtcctccggctacaactacctggactggtatctgcagaagcccggccagtctcctcagctgctgatctactttggctcctccagagcctctggcgtgcccgatagattttctggctctggctccggcaccgacttcaccctgaagatctctagagtggaagccgaggacgtgggcgtgtactactgtatgcagcggagaagaaccccttggtctttcggcggaggcaccaaggtggaaatcaagagaaccgtggctgccccttccgtgttcatcttcccaccatctgacgagcagctgaagtctggcaccgcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgctgatagctcgag

(19) United States
(12) Patent Application Publication
(10) Pub. No.: US 2026/0102505 A1
(43) Pub. Date: Apr. 16, 2026

(54) ANTI-CD45-IGN ANTIBODY DRUG CONJUGATES AND USES THEREOF

(71) Applicant: Vor Biopharma Inc., Cambridge, MA (US)

(72) Inventors: Rahul Palchaudhuri, Somerville, MA (US); Lisa Olson, Hopkinton, MA (US); Kirk Bertelsen, Doylestown, PA (US); Leanne Lanieri, Concord, MA (US); Bindu M. Bennet, Hopkinton, MA (US)

(21) Appl. No.: 19/085,767

(22) Filed: Mar. 20, 2025

Related U.S. Application Data
(63) Continuation of application No. PCT/US2023/074713, filed on Sep. 20, 2023.
(60) Provisional application No. 63/376,435, filed on Sep. 20, 2022.
(60) Provisional application No. 63/483,923, filed on Feb. 8, 2023.
(60) Provisional application No. 63/485,200, filed on Feb. 15, 2023.
(60) Provisional application No. 63/499,340, filed on May 1, 2023.

(51) Int. Cl. A61K 47/68; A61P 35/02

(52) U.S. Cl. CPC A61K 47/6803; A61K 47/6849; A61P 35/02

(57) ABSTRACT

Anti-CD45-IGN Antibody Drug Conjugates and Uses Thereof Disclosed are anti-CD45 antibodies and anti-CD45 antibodies conjugated to IGN cytotoxins that specifically bind to human CD45. Such antibodies and ADCs are useful in therapeutic methods, including methods of depleting CD45+ cells from a patient. The compositions and methods described herein can be used to treat a disorder directly, for instance, by depleting a population of CD45+ cancer cells or autoimmune cells. The compositions and methods described herein can also be used to prepare a patient for hematopoietic stem cell transplant therapy, and to improve the engraftment of hematopoietic stem cell transplants, by selectively depleting endogenous CD45+ cells prior to the transplant procedure.

### CROSS REFERENCE TO RELATED APPLICATIONS

This application is a continuation of International Application No. PCT/US2023/074713, filed Sep. 20, 2023 which claims priority to U.S. Provisional Application No. 63/376,435, filed on Sep. 20, 2022, U.S. Provisional Application No. 63/483,923, filed on Feb. 8, 2023, U.S. Provisional Application No. 63/485,200, filed on Feb. 15, 2023, and U.S. Provisional Application No. 63/499,340, filed on May 1, 2023, the entireties of which are incorporated by reference herein.

### FIELD OF THE INVENTION

Described herein are anti-CD45 antibody drug conjugates (ADCs) for use in the treatment of patients suffering from various pathologies, such as blood diseases and hemoglobinopathies.

### SEQUENCE LISTING

The instant application contains a Sequence Listing which has been submitted electronically in XML format and is hereby incorporated by reference in its entirety. Said XML copy, created on Sep. 19, 2023, is named V118216_2540WO_SL.XML and is 182,724 bytes in size.

### BACKGROUND OF THE INVENTION

CD45, also known as protein tyrosine phosphatase, receptor type C (PTPRC), is an enzyme that, in humans, is encoded by the PTPRC gene (Kaplan et al., PNAS 87:7000-7004 (1990)). CD45 is a member of the protein tyrosine phosphatase (PTP) family, which includes signaling molecules that regulate a variety of cellular processes including cell growth, differentiation, mitotic cycle, and oncogenic transformation. CD45 contains an extracellular domain, a single transmembrane segment, and two tandem intracytoplasmic catalytic domains, and thus belongs to the receptor type PTP family. CD45 is a type I transmembrane protein that is present in various isoforms on differentiated hematopoietic cells (except erythrocytes and plasma cells) (Holmes, Immunology 117:145-55 (2006)). CD45 has been shown to be a regulator of T- and B-cell antigen receptor signaling. It functions through either direct interaction with components of the antigen receptor complexes via its extracellular domain (a form of co-stimulation), or by activating various Src family kinases required for the antigen receptor signaling via its cytoplasmic domain. CD45 also suppresses JAK kinases, and thus functions as a negative regulator of cytokine receptor signaling.

CD45 is present on the surface of hematopoietic cells, including HSCs, leukocytes, and osteoclasts, which are of hematopoietic origin (Shivtiel et al., J Exp Med 205:2381 (2008)). Deletion mutations within CD45 in humans are associated with severe immunodeficiency. This is primarily due to the absence of CD45 on T cells, where it is typically abundant and required to modulate SFK activity during antigen responses. CD45-deficient (CD45^{-/-}) mouse bone marrow contains normal numbers of hematopoietic cells, but the most primitive HSCs are reduced in number, and their mobilization in response to G-CSF is impaired. In part, this defect is intrinsic to the HSC; without CD45-mediated downregulation of SFK activity, integrin-mediated adhesion is high and HSCs are more likely to remain in the stem cell niche. CD45^{-/-} HSCs are also deficient in G-CSF-stimulated mobilization and homing to the chemokine CXCL12/SDF-1, which negatively affects cell engraftment following transplantation. These deficiencies can be restored by supplementation with SFK inhibitors, indicating that this role is usually performed by CD45. Likewise, CD45^{-/-} recipients also show deficient engraftment and subsequent mobilization of normal HSCs, indicating a role for CD45 in the stem cell niche, as well as in the HSC (Shivtiel et al., J Exp Med 205:2381 (2008)).

Despite advances in the medicinal arts, there remains a demand for treating pathologies of the hematopoietic system, such as diseases of a particular blood cell, metabolic disorders, cancers, and autoimmune conditions, among others. While hematopoietic stem cells (HSCs) have significant therapeutic potential, a limitation that has hindered their use in the clinic has been the difficulty associated with ensuring engraftment of HSC transplants in a host. In particular, hematopoietic stem cell therapies involving antibodies that target cell surface antigens on endogenous HSCs can trigger unwanted immunostimulatory and effector functions that impede engraftment of an exogenous HSC transplant. As CD45 is expressed, for example, on HSCs and leukocytes, it presents a target for therapies including conditioning therapies, immune reset, and treatment of diseases.

### SUMMARY OF THE INVENTION

Given the important role of CD45 in cell biology, there is a need for anti-CD45 antibodies, and fragments thereof. Described herein are anti-CD45 antibodies, antigen binding fragments thereof, and antibody drug conjugates (ADCs) thereof. The anti-CD45 antibodies, antigen binding fragments thereof, and ADCs thereof may bind to hematopoietic stem cells (HSCs) and are useful, for example, as conditioning agents for HSC transplantation. In particular, the anti-CD45 antibodies, antigen binding fragments thereof, and ADCs thereof described herein can be used to specifically deplete, for example, host HSCs, immune cells (e.g., leukocytes), or disease-causing cells. Further, the anti-CD45 antibodies, antigen binding fragments thereof, and ADCs thereof described herein may be used to treat patients with a leukemia or a lymphoma, or to treat patients with hemoglobinopathy disorders, including, without limitation, sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S-C disease, Fanconi anemia, aplastic anemia, or Wiskott-Aldrich syndrome. The anti-CD45 antibodies, antigen binding fragments thereof, and ADCs described herein satisfy a need for compositions and methods for promoting the engraftment of exogenous hematopoietic stem cell grafts such that the multi-potency and hematopoietic functionality of these cells is preserved following transplantation.

In a first aspect, the present disclosure provides a method of depleting CD45+ cells in a human subject in need of a HSC transplant, the method comprising administering to the human subject, wherein the human subject has relapsed and/or refractory acute myeloid leukemia (AML) or recurrent T-cell lymphoma, a single dose of at least 0.15 mg/kg of an ADC, comprising an anti-CD45 antibody conjugated to an indolinobenzodiazepine (IGN) cytotoxin via a linker. In some embodiments, the anti-CD45 antibody comprises: a heavy chain variable region comprising a CDR1 domain comprising the amino acid sequence as set forth in SEQ ID NO:42, a CDR2 domain comprising the amino acid sequence as set forth in SEQ ID NO:43, and a CDR3 domain comprising the amino acid sequence as set forth in SEQ ID NO:44, and a light chain variable region comprising a CDR1 domain comprising the amino acid sequence as set forth in SEQ ID NO:46, a CDR2 domain comprising the amino acid sequence as set forth in SEQ ID NO:47, and a CDR3 domain comprising the amino acid sequence as set forth in SEQ ID NO:48.

In another aspect, the present disclosure provides a method of conditioning a human subject in need of a hematopoietic stem cell (HSC) transplant, the method comprising administering to a human subject a single dose of at least 0.15 mg/kg of an ADC, comprising an anti-CD45 antibody conjugated to an IGN cytotoxin via a linker. In some embodiments, the anti-CD45 antibody comprises: a heavy chain variable region comprising a CDR1 domain comprising the amino acid sequence as set forth in SEQ ID NO:42, a CDR2 domain comprising the amino acid sequence as set forth in SEQ ID NO:43, and a CDR3 domain comprising the amino acid sequence as set forth in SEQ ID NO:44, and a light chain variable region comprising a CDR1 domain comprising the amino acid sequence as set forth in SEQ ID NO:46, a CDR2 domain comprising the amino acid sequence as set forth in SEQ ID NO:47, and a CDR3 domain comprising the amino acid sequence as set forth in SEQ ID NO:48.

In some embodiments, the method further comprises administering an HSCT to the human subject.

In yet another aspect, the present disclosure provides a method of conditioning a human subject having a disease or disorder, the method comprising administering to the human subject a single dose of at least 0.15 mg/kg of an ADC, wherein the human subject has a disorder treatable with the transplant of genetically-modified cells, comprising an anti-CD45 antibody conjugated to an IGN cytotoxin via a linker. In some embodiments, the anti-CD45 antibody or antigen binding portion thereof comprises: a heavy chain variable region comprising a CDR1 domain comprising the amino acid sequence as set forth in SEQ ID NO:42, a CDR2 domain comprising the amino acid sequence as set forth in SEQ ID NO:43, and a CDR3 domain comprising the amino acid sequence as set forth in SEQ ID NO:44, and a light chain variable region comprising a CDR1 domain comprising the amino acid sequence as set forth in SEQ ID NO:46, a CDR2 domain comprising the amino acid sequence as set forth in SEQ ID NO:47, and a CDR3 domain comprising the amino acid sequence as set forth in SEQ ID NO:48.

In some embodiments, the disorder treatable with the transplant of genetically-modified cells is a hemoglobinopathy disorder. In some embodiments, the hemoglobinopathy disorder is selected from the group of sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S-C disease, Fanconi anemia, aplastic anemia, or Wiskott-Aldrich syndrome.

In some embodiments, the genetically-modified cells are modified to express wildtype hemoglobin.

In some embodiments of the aforementioned aspects, the ADC is administered to a human subject at a single dose of 0.15-0.4 mg/kg. In some embodiments, the ADC is administered to the human subject at a single dose of about 0.15 mg/kg. In some embodiments, the ADC is administered to the human subject at a single dose of about 0.2 mg/kg. In some embodiments, the ADC is administered to the human subject at a single dose of about 0.3 mg/kg.

In one embodiment, the transplant (e.g., HSCs or genetically-modified cells) is administered to the human subject about 10 days after the ADC is administered.

In some embodiments of the aforementioned aspects, the present disclosure provides an anti-CD45 antibody comprising: a heavy chain variable region comprising the amino acid sequence as set forth in SEQ ID NO:41, and a light chain variable region comprising the amino acid sequence as set forth in SEQ ID NO:45.

In some embodiments of the aforementioned aspects, the isolated anti-CD45 antibody comprises an Fc region. In certain embodiments, the Fc region is a human IgG1 Fc region of a human IgG4 Fc region.

In some embodiments, the antibody is a monoclonal antibody.

In some embodiments, the antibody is an intact antibody comprising a constant region.

In some embodiments, the antibody is an IgG. In particular embodiments, the IgG is an IgG1 or an IgG4.

In some embodiments of the aforementioned aspects, the antibody comprises a constant region, wherein the constant region comprises at least one, at least two, at least three, at least four, or at least five amino acid substitutions selected from the group consisting of L234A, L235A, D265C, H310A, and H435A (numbering according to the EU index). In particular embodiments, the constant region comprises amino acid substitutions L234A, L235A and D265C (numbering according to the EU index).

In some embodiments, the isolated anti-CD45 antibody of the present disclosure comprises a constant region, wherein the constant region comprises (a) a heavy chain amino acid sequence as set forth in SEQ ID NO:102, SEQ ID NO:103, SEQ ID NO:104, SEQ ID NO:105, or SEQ ID NO:106; and (b) a light chain amino acid sequence as set forth in SEQ ID NO:101.

In some embodiments of the aforementioned aspects, the present disclosure provides an anti-CD45 antibody, comprising: a heavy chain amino acid sequence as set forth in SEQ ID NO:49, and a light chain amino acid sequence as set forth in SEQ ID NO:50.

In some embodiments of the ADC disclosed herein, the cytotoxin is a DNA alkylating agent. In some embodiments the cytotoxin is indolinobenzodiazepine (IGN), an IGN dimer, or an IGN pseudo dimer. In some embodiments, the IGN is DGN549.

In some embodiments, the cytotoxin is an IGN pseudo dimer represented by:

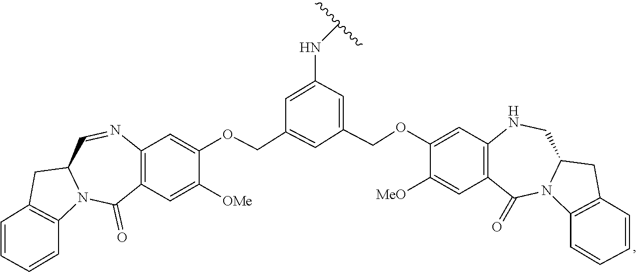 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the point of covalent attachment to the linker of the ADC.

In some embodiments, the linker comprises a dipeptide, a disulfide, C1-C12 alkyl, C═O, or combinations thereof.

In some embodiments, the linker comprises

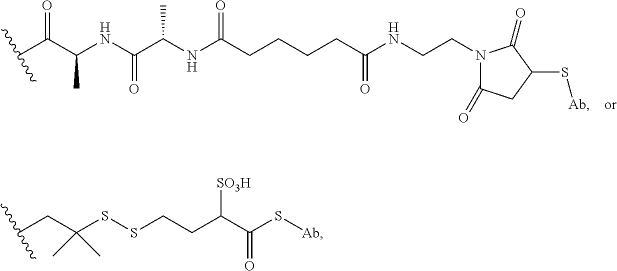

wherein the wavy line indicates the point of attachment to a cytotoxin and Ab is an anti-CD45 antibody.

In some embodiments, the cytotoxin-linker conjugate, prior to conjugation to the anti-CD45 antibody or antigen binding portion thereof, and including the reactive substituent Z′, taken together as Cy-L-Z′, has a structure of:

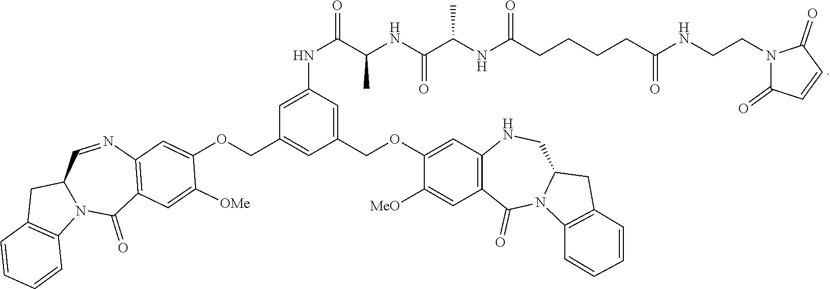 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

In one aspect, the ADC has a formula:

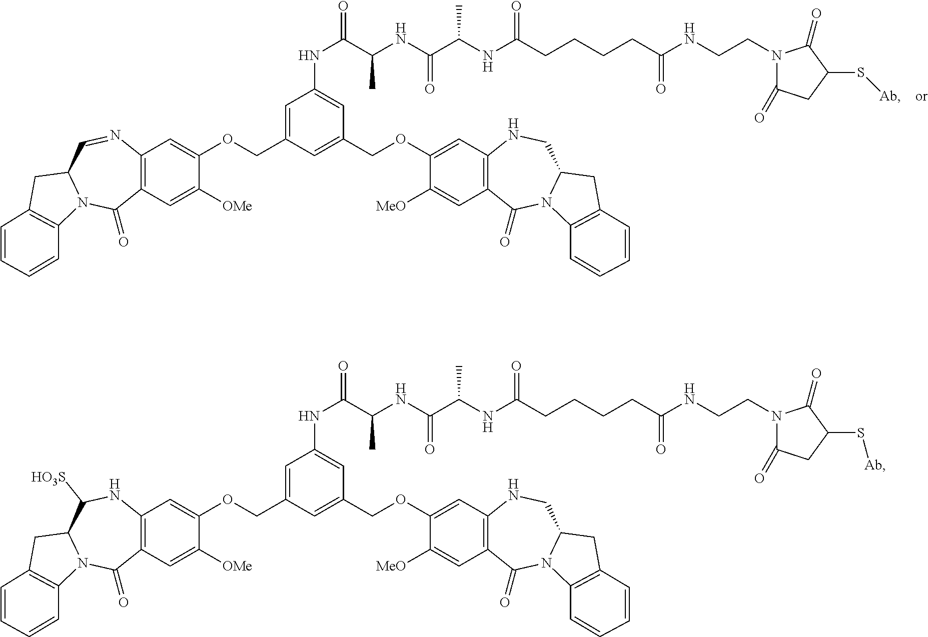

wherein Ab is the anti-CD45 antibody.

Also provided herein is a method of conditioning a human subject in need thereof, the method comprising administering to the human subject a dose of 0.15 mg/kg to 0.4 mg/kg of an antibody-drug conjugate (ADC) comprising an indolinobenzodiazepine pseudo dimer conjugated to an anti-CD45 antibody (Ab) via a linker, such that the human subject is conditioned for a stem cell transplant, wherein the ADC has a formula

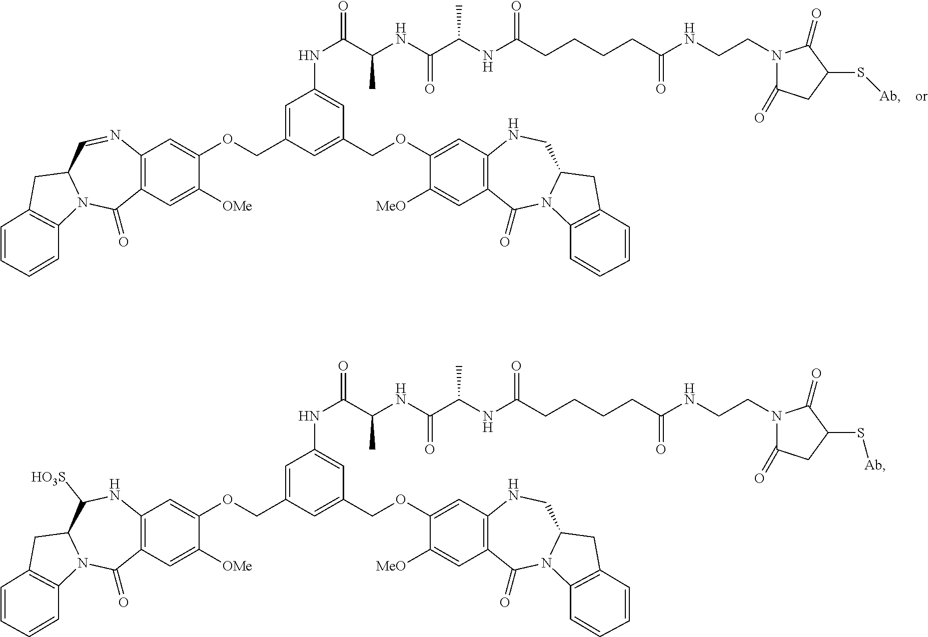

wherein the anti-CD45 antibody comprises a heavy chain variable region comprising a CDR1 domain comprising the amino acid sequence as set forth in SEQ ID NO:42, a CDR2 domain comprising the amino acid sequence as set forth in SEQ ID NO:43, and a CDR3 domain comprising the amino acid sequence as set forth in SEQ ID NO:44; comprises a light chain variable region comprising a CDR1 domain comprising the amino acid sequence as set forth in SEQ ID NO:46, a CDR2 domain comprising the amino acid sequence as set forth in SEQ ID NO:47; and a CDR3 domain comprising the amino acid sequence as set forth in SEQ ID NO:48; and is an IgG1 isotype.

Further provided is a method of conditioning a human subject in need thereof, the method comprising administering to the human subject a dose of 0.15 mg/kg to 0.4 mg/kg of an antibody-drug conjugate (ADC) comprising an indolinobenzodiazepine pseudo dimer conjugated to an anti-CD45 antibody (Ab) via a linker, such that the human subject is conditioned for a stem cell transplant, wherein the ADC has a formula

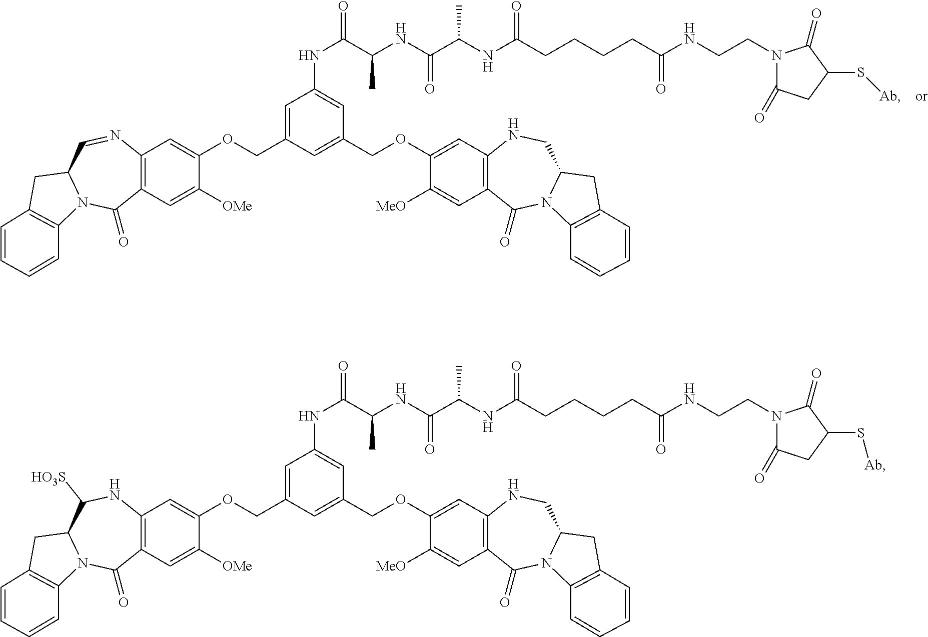

wherein the anti-CD45 antibody comprises a heavy chain variable region comprising an amino acid sequence as set forth in SEQ ID NO: 41, and a light chain variable region comprising an amino acid sequence as set forth in SEQ ID NO: 45, and is an IgG1 isotype.

In addition, provided herein is a method of conditioning a human subject in need thereof, the method comprising administering to the human subject a dose of 0.15 mg/kg to 0.4 mg/kg of an antibody-drug conjugate (ADC) comprising an indolinobenzodiazepine pseudo dimer conjugated to an anti-CD45 antibody (Ab) via a linker, such that the human subject is conditioned for a stem cell transplant, wherein the ADC has a formula

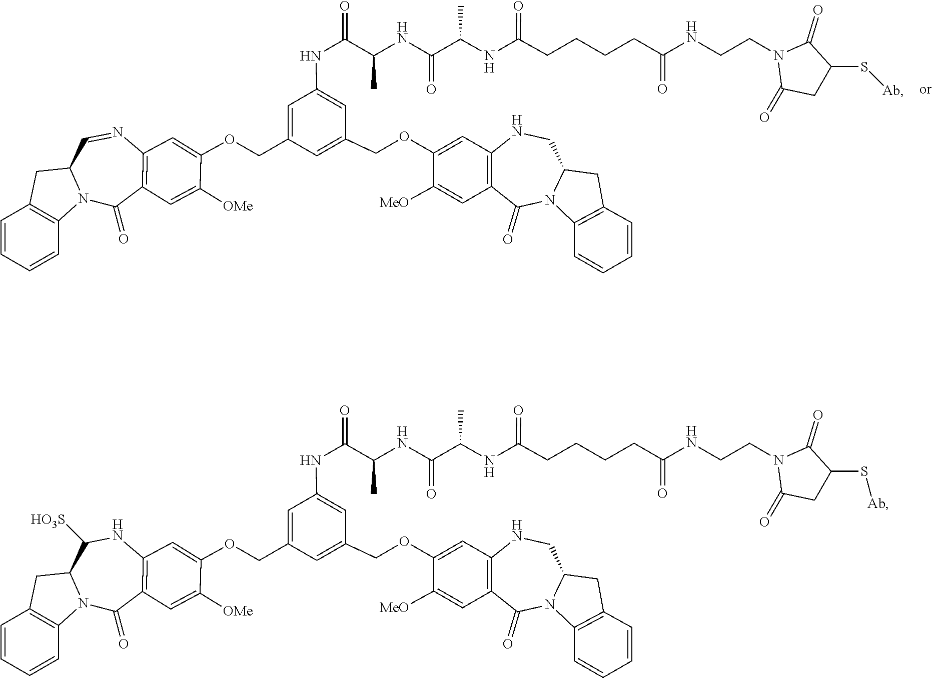

wherein the anti-CD45 antibody comprises a heavy chain amino acid sequence as set forth in SEQ ID NO: 49, and a light chain amino acid sequence as set forth in SEQ ID NO: 50, and is an IgG1 isotype.

In certain embodiments, the ADC is administered to the human subject at a dose of 0.15 mg/kg.

In other embodiments, the ADC is administered to the human subject at a dose of 0.2 mg/kg.

In still other embodiments, the ADC is administered to the human subject at a dose of 0.3 mg/kg.

In further embodiments, the ADC is administered to the human subject at a dose of about 0.17 mg/kg.

In yet other embodiments, the ADC is administered to the human subject at a dose of about 0.22 mg/kg.

In one embodiment, the ADC is administered to the human subject at a dose of about 0.37 mg/kg.

In certain embodiments, a single dose of the ADC is administered to the human subject.

In other embodiments, the ADC is a monotherapy conditioning agent.

In some embodiments, the human patient has a hematological cancer.

In other embodiments, the human patient has acute myeloid leukemia (AML), myeloproliferative neoplasm (MPN), or myelodysplastic syndrome (MDS).

In certain embodiment, the method further comprises administering an HSC transplant to the human patient. In certain embodiments, the HSC transplant is administered at least 14 days after administration of the ADC to the human patient.

In another aspect, the present disclosure provides a pharmaceutical composition comprising an ADC described hereinabove, and a pharmaceutically acceptable carrier.

In one embodiment, the methods disclosed herein can be used with ADCs comprising any of the antibodies disclosed in Table 3 and the sequence listing.

### BRIEF DESCRIPTION OF THE FIGURES

FIG. 1 depicts a multiple sequence alignment of the heavy chain variable regions and the light chain variable regions of anti-CD45 antibodies Ab1, Ab2, Ab3, Ab4, Ab5, Ab6, and Ab7. The CDRs of each variable region are indicated in bold type.

FIG. 2 shows the cytotoxicity of an anti-CD45-ADC on human stem cells and human peripheral blood mononuclear cells in vitro as compared to an isotype-ADC control, a naked CD45 antibody, and free payload.

FIG. 3A-C demonstrates the effect of a single anti-CD45-ADC treatment on HSC and peripheral blood cell depletion in a humanized mouse model. FIG. 3A presents an overview of the experimental design. FIG. 3B shows the depletion of both human stem cells and human peripheral cells following treatment with an anti-CD45-ADC as compared to isotype-ADC and PBS controls. FIG. 3C shows the depletion of immune cell types, (T, B, and Monocytes) following treatment with an anti-CD45-ADC as compared to an isotype-ADC control.

FIG. 4A-C shows the long-term, disease-free survival in a patient-derived xenograft (PDX) model for AML across multiple treatment cohorts. Mice were treated with various doses of either an anti-CD45-ADC or controls, including isotype-ADC, CD45 mAb, PBS, or the standard of care (ARA-C). FIG. 4A shows the percent survival over time post-inoculation in days. FIG. 4B depicts the percentage of tumor burden over time post-inoculation in days. FIG. 4C provides the median survival in days for each treatment group.

FIG. 5A-C shows the long-term, disease-free survival in a PDX model for recurrent angioimmunoblastic T-cell lymphoma across multiple treatment cohorts. Mice were treated with various doses of either an anti-CD45-ADC, or controls, including isotype-ADC, CD45 mAb, PBS, ARA-C, or Dexamethasone. FIG. 5A depicts the percent tumor growth over time. FIG. 5B shows the percent survival over time. FIG. 5C provides the median survival across treatment groups.

FIG. 6A-D demonstrate the effects of a single anti-CD45-ADC treatment in a Cynomolgus monkey model. FIG. 6A presents an overview of the experimental design. FIG. 6B shows the effects of the anti-CD45-ADC on depleting HSCs. FIG. 6C shows the effects of the anti-CD45-ADC on depleting immune cells. FIG. 6D shows the effects of the anti-CD45-ADC on immune cell function.

FIG. 7 summarizes the pharmacokinetics of the anti-CD45-ADC in a Cynomolgus monkey model at various doses administered via either infusion or bolus.

FIG. 8A-E demonstrate the effects of a single anti-CD45-ADC treatment in a Cynomolgus monkey model. FIG. 8A presents an overview of the experimental design. FIG. 8B shows the depletion of the anti-CD45-ADC on bone marrow HSCs at days 6 or 7 post-dose as compared with a Busulfan control. FIG. 8C shows the level of peripheral B cells after treatment relative to pre-dose over time. FIG. 8D shows the level of peripheral T cells after treatment relative to pre-dose over time. FIG. 8E monitors T cell expansion ex vivo both pre- and post-dose.

FIG. 9A-C demonstrates the effects of an anti-CD45-ADC in a Rhesus Macaque model. FIG. 9A presents an overview of the experimental design. FIG. 9B-C show the effects of anti-CD45-ADC treatment on HSC depletion (FIG. 9B) and immune cell function (FIG. 9C) at various doses.

FIG. 10A-D shows various clinical pathology parameters assessed in Rhesus Macaques following treatment with an anti-CD45-ADC. FIG. 10A depicts the levels of aspartate aminotransferase (AST). FIG. 10B depicts the levels of alanine aminotransferase (ALT). FIG. 10C depicts the levels of bilirubin. FIG. 10D depicts the levels of alkaline phosphatase (ALP).

FIG. 11A-B demonstrates the ability for anti-CD45-ADC to enable edited cell engraftment (FIG. 11A) and induce F-cells (FIG. 11B) in a Rhesus Macaque model as compared to the myeloablative Busulfan.

FIG. 12 depicts the chimerism of GFP+ labeled cells following conditioning and engraftment segmented by myeloid and lymphoid.

FIGS. 13A-C graphically depict the results of an assay evaluating the Minimum Efficacious Dose (MED) for HSC+Immune Cell Depletion in NHP using an anti-CD45-DGN549 ADC.

FIGS. 14A-D. An anti-CD45-ADC demonstrated robust depletion of bone marrow HSCs in cynomolgus macaques (FIG. 14A) and allows engraftment of erythroid-specific BCL11A enhancer-edited CD34+ cells (FIG. 14B). Engrafted cells showed robust HbF induction as measured by HbF positive cells (FIGS. 14C, 14D).

FIGS. 15A-F shows that an anti-CD45-ADC enables engraftment of edited CD34+ hematopoietic stem and progenitor cells. FIG. 15A provides an overview of the experimental design. FIGS. 15B and 15E show the overall percentage of engrafted edited cells in peripheral granulocytes over the course of 80 weeks (FIG. 15B) and 200 weeks (FIG. 15E) after transplantation. FIG. 15C-D depict robust induction of fetal hemoglobin levels following anti-CD45-ADC treatment as measured by the level of F-cells (FIG. 15C) and γ-globin levels (FIG. 15D). FIG. 15F shows the overall percentage of engrafted edited cells in peripheral blood mononuclear cells over 200 weeks.

FIG. 16 graphically depicts indel frequencies in (expressed as overall editing %) in the indicated bone marrow cell subsets in rhesus macaques treated with a single dose of anti-CD45-ADC (Ab5-DGN549) at either 0.2 or 0.3 mg/kg followed by engraftment of erythroid-specific BCL11A enhancer-edited CD34+ cells.

FIG. 17 graphically depicts Shannon diversity indices of peripheral blood granulocytes (PB Gr) compared across anti-CD45-ADC, busulfan, and TBI conditioned macaques.

FIG. 18 provides a reaction scheme for chemical conjugation of an anti-CD45 antibody via a succinimide linkage to DGN549-C (DGN549 with a protease-cleavable linker).

FIG. 19 depicts sulfonated DGN549-C (DGN549 with a protease-cleavable linker) conjugated to an anti-CD45 antibody. The anti-CD45 ADC in FIG. 19 has a drug-to-antibody ratio of 2.

### DETAILED DESCRIPTION

Described herein are antibody-drug conjugates (ADCs) capable of binding CD45 that can be used as therapeutic agents to, for example, condition and promote the engraftment of transplanted HSCs in a patient in need of transplant therapy and/or treat cancers and autoimmune diseases characterized by CD45+ cells. These therapeutic activities can be caused, for instance, by the binding of isolated anti-CD45 antibodies that bind to CD45 expressed on the surface of a cell, such as a cancer cell, or hematopoietic stem cell, and subsequently inducing cell death via the toxin, e.g., DGN549, to which the antibody is conjugated via a linker.

Disclosed herein are anti-CD45 antibody-drug conjugates (ADCs) that can be used to treat patients with conditions for which depletion of CD45+ cells is beneficial, including, but not limited to, leukemias and lymphomas, as well as patients with autoimmune diseases such as multiple sclerosis and scleroderma. In addition, the anti-hematopoietic cell antibodies (anti-CD45 antibodies) included herein are useful in hematopoietic stem cell therapies. For example, the ADCs described herein are useful in conditioning procedures, in which a patient is prepared for receipt of a transplant including hematopoietic stem cells and/or genetically-modified cells. Such conditioning methods promote the engraftment of the stem cell transplant (e.g., a hematopoietic stem cell transplant). According to the methods described herein, a patient may be conditioned for a stem cell transplant therapy (e.g., a hematopoietic stem cell transplant therapy and/or a transplant with genetically-modified cells) by administration to the patient of an ADC capable of binding CD45 (e.g., CD45 expressed by hematopoietic cells (e.g., hematopoietic stem cells) or mature immune cells (e.g., T cells). As described herein, an anti-CD45 antibody may be covalently conjugated to a cytotoxin so as to form an antibody drug conjugate (ADC). Administration of an ADC capable of binding CD45 to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

The sections that follow provide methods for administering certain doses of an anti-CD45 ADC comprising an anti-CD45 conjugated via a linker to an IGN, e.g., DGN549, to a human patient, such as a patient suffering from a cancer or autoimmune disease, or a patient in need of a stem cell transplant therapy (e.g., a hematopoietic stem cell transplant therapy and/or a transplant therapy with genetically-modified cells) in order to promote engraftment of the transplant (e.g., hematopoietic stem cell grafts and/or genetically-modified cells), as well as methods of administering such therapeutics to a patient (e.g., prior to hematopoietic stem cell transplantation and/or a transplantation with genetically-modified cells).

### Definitions

As used herein, the term “about” refers to a value that is within 5% above or below the value being described. For example, the term “about 100 nM” indicates a range of 95-105 nM.

As used herein, the term “allogeneic”, in the context of transplantation, is used to define a transplant (e.g., cells, tissue or an organ transplant) that is transplanted from a donor to a recipient, wherein the recipient is a different individual of the same species, relative to the donor.

As used herein, the term “autologous”, in the context of transplantation, refers to a transplant where the donor and recipient are the same individual, i.e., the same subject.

As used herein, the term “xenogeneic”, in the context of transplantation, refers to a transplant where the donor and recipient are of different species.

As used herein, the term “immune cell” is intended to include, but is not limited to, a cell that is of hematopoietic origin and that plays a role in the immune response.

Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), and antibody fragments so long as they exhibit the desired antigen-binding activity.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding fragment,” or “antigen binding portion” of an antibody, as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)2, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment containing two Fab fragments linked by a disulfide bridge at the hinge region; (iii) a Fv fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

An “intact” or “full length” antibody, as used herein, refers to an antibody having two heavy (H) chain polypeptides and two light (L) chain polypeptides interconnected by disulfide bonds. In certain embodiments, a toxin can be conjugated to an intact anti-CD45 antibody having heavy and/or light chain amino acid sequences described herein.

The term “monoclonal antibody” as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof.

The terms “Fc region,” “Fc domain,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the entire second constant domain CH2 (residues at EU positions 231-340 of IgG1) and the third constant domain CH3 (residues at EU positions 341-447 of human IgG1). As used herein, the Fc domain includes the “lower hinge region” (residues at EU positions 233-239 of IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or Clq as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. It is noted that the order in which substitutions are provided is arbitrary.

The terms “Fc gamma receptor” or “Fc gamma R” as used herein refer to any member of the family of proteins that bind the IgG antibody Fc region and are encoded by the FcgammaR genes. In humans this family includes but is not limited to FcgammaRI (CD64), including isoforms FcgammaRIa, FcgammaRIb, and FcgammaRIc; FcgammaRII (CD32), including isoforms FcgammaRIIa (including allotypes H131 and R131), FcgammaRIIb (including FcgammaRIIb-1 and FcgammaRIIb-2), and FcgammaRIIc; and FcgammaRIII (CD16), including isoforms FcgammaRIIIa (including allotypes V158 and F158) and FcgammaRIIIb (including allotypes FcgammaRIIIb-NA1 and FcgammaRIIIb-NA2). An FcgammaR can be from any organism, including but not limited to humans, mice, rats, rabbits, and monkeys. Mouse FcgammaRs include but are not limited to FcgammaRI (CD64), FcgammaRII (CD32), FcgammaRIII (CD16), and FcgammaRIII-2 (CD16-2).

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma-delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, H435A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

As used herein, the terms “condition” and “conditioning” refer to processes by which a patient is prepared for receipt of a transplant, e.g., a transplant containing hematopoietic stem cells and/or genetically-modified cells. Such procedures promote the engraftment of, e.g., a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC comprising an antibody or antigen-binding fragment thereof capable of binding CD45 expressed by hematopoietic stem cells. As described herein, the antibody may be covalently conjugated to a cytotoxin so as to form a drug-antibody conjugate. Administration of an ADC capable of binding the foregoing antigen to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “effective amount” or “therapeutically effective amount” refers to an amount of a therapeutic agent, e.g., an anti-CD45 ADC, that is sufficient to achieve the desired result in the context of treating, preventing, ameliorating, or reducing the symptoms of a disease or disorder in a patient. For example, in some embodiments, a therapeutically effective amount of an anti-CD45 antibody or ADC is an amount sufficient to reduce or deplete a population of CD45+ cells in a patient. In other embodiments, a therapeutically effective amount of an anti-CD45 antibody or ADC is an amount sufficient to condition a patient for receipt of a hematopoietic stem cell transplant. In such embodiments, the therapeutically effective amount can be, for example, an amount sufficient to selectively deplete endogenous hematopoietic stem cells from the patient, and/or an amount sufficient to promote the engraftment of a hematopoietic stem cell transplant in the patient. In other embodiments, a therapeutically effective amount of an anti-CD45 antibody or ADC is an amount sufficient to have an effect on an autoimmune disease or cancer in a human patient.

As used herein, the term “half-life” refers to the time it takes for the plasma concentration of the antibody drug in the body to be reduced by one half or 50% in a subject, e.g., a human subject. This 50% reduction in serum concentration reflects the amount of drug circulating.

As used herein, the phrase “substantially cleared from the blood” refers to a point in time following administration of a therapeutic agent (such as an anti-CD45 antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, or antibody fragments, such as ELISA-based detection assays known in the art or described herein. Additional assays that can be used to detect antibodies, or antibody fragments, include immunoprecipitation techniques and immunoblot assays, among others known in the art.

The terms “specific binding” or “specifically binding”, as used herein, refers to the ability of an antibody to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD45, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD45” or “specifically binds to CD45,” as used herein, refers to an antibody or that binds to CD45 and has a dissociation constant (K_{D}) of 1.0×10⁻⁷ M or less, as determined by surface plasmon resonance. In one embodiment, K_{D} (M) is determined according to standard bio-layer interferometry (BLI). In one embodiment, K_{off} (1/s) is determined according to standard bio-layer interferometry (BLI). It shall be understood, however, that the antibody may be capable of specifically binding to two or more antigens which are related in sequence. For example, in one embodiment, an antibody can specifically bind to both human and a non-human (e.g., mouse, cynomolgus or non-human primate) orthologs of CD45. Thus, as used herein, an antibody that “specifically binds to human CD45” is intended to refer to an antibody that binds to human CD45 (and possibly CD45 from one or more non-human species, such as cynomolgus) but does not substantially bind to non-CD45 proteins. Preferably, the antibody binds to human CD45 with a K_{D} of 1×10⁻⁷ M or less, a K_{D} of 5×10⁻⁸ M or less, a K_{D} of 3×10⁻⁸ M or less, a K_{D} of 1×10⁻⁸ M or less, or a K_{D} of 5×10⁻⁹ M or less.

As used herein, the term “human antibody” is intended to include antibodies having variable regions derived from human germline immunoglobulin sequences. In embodiments in which a human antibody contains a constant region, the constant region can likewise be derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Human antibodies can be made by a variety of methods known in the art including phage display methods or yeast display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

“Humanized” forms of non-human (e.g., murine or rat) antibodies are immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise all or a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

Also provided are “conservative sequence modifications” of the sequences set forth in SEQ ID NOs described herein. Conservative sequence modifications include nucleotide and amino acid sequence modifications which do not abrogate the binding of an antibody or antigen binding portion thereof containing an amino acid sequence, encoded by a nucleotide sequence, provided herein to its cognate antigen (e.g., CD45). Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD73 antibody is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “hematopoietic stem cells” (“HSCs”) refers to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B cells and T cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, T cells and B cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the terms “subject” and “patient” refer to an organism, such as a human, that receives treatment for a particular disease or condition as described herein. In some embodiments, the subject or patient referenced in the methods provided herein is a human subject.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein “to treat” or “treatment”, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. For example, treatment can refer to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease. Beneficial or desired clinical results include, but are not limited to, promoting the engraftment of exogenous hematopoietic cells in a patient following antibody conditioning therapy as described herein and subsequent hematopoietic stem cell transplant therapy Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD45+ leukemic cells) or autoimmune cells (e.g., CD45+ autoimmune lymphocytes, such as a CD45+ T-cell that expresses a T-cell receptor that cross-reacts with a self antigen). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S-C disease, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome (MDS). In some embodiments, the subject has a myeloproliferative neoplasm (MPN). In some embodiments, the subject has blastic plasmacytoid dendritic cell neoplasm (BPDCN). In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias (e.g., beta thalassemia), Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, B-cell acute lymphoblastic leukemia (B-ALL), acute lymphoid leukemia, chronic myeloid leukemia, chronic lymphoid leukemia, multiple myeloma, diffuse large B-cell lymphoma, non-Hodgkin's lymphoma, multiple myeloma (MM), myelodysplastic syndrome (MDS), or blastic plasmacytoid dendritic cell neoplasm (BPDCN). In some embodiments, the hematopoietic malignancy is acute myeloid leukemia (AML). In some embodiments, the hematopoietic malignancy is multiple myeloma (MM). In some embodiments, the hematopoietic malignancy is myelodysplastic syndrome (MDS). In some embodiments, the hematopoietic malignancy is a myeloproliferative neoplasm (MPN). Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the disclosure include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “conjugate”, “cytotoxin-linker conjugate”, “antibody drug conjugate” or “ADC” refers to an antibody which is linked to a cytotoxin or toxin via a linker. The terms are used interchangeably throughout in reference to such a molecule. In one embodiment, an ADC is formed by the chemical bonding of a reactive functional group of one molecule, such as an antibody or antigen-binding fragment thereof, with an appropriately reactive functional group of another molecule, such as a cytotoxin described herein. Non-limiting examples of cytotoxins that can, in some embodiments, be used in a conjugate provided herein include a small organic molecule (e.g., MW 1500 Da or less), a biomolecule (e.g., a protein), a drug filled nanoparticle, or a radionuclide. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

The term “conjugate”, “conjugate to” or “conjugate with”, when used in the sense of at least two molecules being conjugated together, refers to one molecule, e.g., an antibody, being linked to or combined with a second molecule, e.g., a toxin. Anti-CD45 antibodies, and fragments thereof, can be conjugated to other molecules, including toxins, labelling agents (e.g., fluorescein or biotin), drug-loaded nanoparticles. Conjugated molecules may be conjugated via covalent or non-covalent interactions. In certain embodiments, an anti-CD45 antibody, or fragment thereof, is conjugated to a protein toxin to form a protein fusion, e.g., an scFv-toxin chimera. In some embodiments, the conjugated molecules can be coupled via the non-covalent interaction of a first interacting moiety (e.g., biotin) and a second interacting moiety (e.g., streptavidin) associated with the conjugated molecules.

### Anti-CD45 Antibody Drug Conjugates (ADCs)

### Anti-CD45 Antibodies

Contemplated herein are antibodies, or antigen-binding fragments thereof, capable of binding CD45, that can be used as therapeutic agents as conjugates (ADCs) to, for example, (i) treat cancers and autoimmune diseases characterized by CD45+ cells and (ii) promote the engraftment of transplanted hematopoietic stem cells in a patient in need of transplant therapy. These therapeutic activities can be caused, for instance, by ADCs that bind to CD45 expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein. Additionally, depletion of leukocytes in a patient in need thereof in combination with HSC transplant can reset the patient's immune system, thereby, for example, curing the patient of an autoimmune disease.

CD45, also known as leukocyte common antigen and receptor-type tyrosine-protein phosphatase C, is a hematopoietic cell-specific transmembrane protein tyrosine phosphatase essential for T and B cell antigen receptor-mediated signaling. CD45 includes a large extracellular domain, and a phosphatase containing cytosolic domain. CD45 may act as both a positive and negative regulator depending on the nature of the stimulus and the cell type involved. Although there are a large number of permutations possible in the CD45 gene, only six isoforms are traditionally identified in humans. The isoforms are RA, RO, RB, RAB, RBC and RABC (Hermiston et al. 2003 “CD45: a critical regulator of signaling thresholds in immune cells.” Annu Rev Immunol. 2:107-137.). CD45RA is expressed on naïve T cells, and CD45RO is expressed on activated and memory T cells, some B cell subsets, activated monocytes/macrophages, and granulocytes. CD45RB is expressed on peripheral B cells, naïve T cells, thymocytes, weakly on macrophages, and dendritic cells. An amino acid sequence of CD45RABC is provided herein as SEQ ID NO:112. An amino acid sequence of CD45RA is provided herein as SEQ ID NO:107. An amino acid sequence of CD45RO is provided herein as SEQ ID NO:108. An amino acid sequence of CD45RB is provided herein as SEQ ID NO:109. An amino acid sequence of CD45RAB is provided herein as SEQ ID NO:110. An amino acid sequence of CD45RBC is provided herein as SEQ ID NO:111.

Certain human antibodies (designated Antibody 1 (Ab1), Antibody 2 (Ab2), Antibody 3 (Ab3), Antibody 4 (Ab4), Antibody 5 (Ab5), Antibody 6 (Ab6), and Antibody 7 (Ab7)) disclosed herein bind to human CD45 (all isoforms), and can cross-react with CD45 from non-human primates (e.g., cynomolgus CD45 and/or rhesus CD45). Other humanized and affinity matured (designated Antibody A (AbA), Antibody B (AbB), and Antibody C (AbC) bind to human CD45 (all isoforms) and cross-react with CD45 from non-human primates (e.g., cynomolgus CD45 and/or rhesus CD45). These antibodies have diagnostic and therapeutic characteristics useful as ADCs and the therapeutics methods described herein.

In one embodiment, an ADC comprises an antibody, or antigen binding portion thereof, that binds to human CD45 (SEQ ID NO:112) and to cynomolgus CD45 (SEQ ID NO:145) and/or to rhesus CD45 (SEQ ID NO:146). In some embodiments, the antibody, of antigen-binding portion thereof, can bind to human CD45 with a K_{D} of about 100 nM or less, e.g., about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 10 nM or less, or about 0.1 nM or less, as determined by Bio-Layer Interferometry (BLI). In some embodiments, the antibody, of antigen-binding portion thereof, can bind to cynomolgus CD45 with a K_{D} of about 100 nM or less, e.g., about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 10 nM or less, or about 0.1 nM or less, as determined by Bio-Layer Interferometry (BLI). In some embodiments, the antibody, of antigen-binding portion thereof, can bind to rhesus CD45 with a K_{D} of about 100 nM or less, e.g., about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 10 nM or less, or about 0.1 nM or less, as determined by Bio-Layer Interferometry (BLI). In some embodiments, the antibody is a fully human antibody, or antigen-binding portion thereof. In other embodiments, the antibody is a humanized antibody, or antigen-binding portion thereof. In some embodiments, the antibody is a chimeric antibody, or antigen-binding portion thereof. In some embodiments, the antibody is a deimmunized antibody, or antigen-binding portion thereof.

The extracellular region of human CD45 includes a mucin-like domain, and four fibronectin-like domains (d1, d2, d3, and d4). Without wishing to be bound by any theory, it is believed that antibodies Ab1, Ab2, Ab3, Ab4, Ab5, Ab6, and Ab7 interact with residues of human CD45 located within the d3 and d4 fibronectin-like domains. In particular, these antibodies may interact with a fragment of human CD45 set forth in SEQ ID NO:115, and a fragment of human CD45 set forth in SEQ ID NO:117. Crosslinking studies described herein suggest that the antibodies can specifically interact with one or more CD45 amino acid residues, which are conserved between human CD45, cynomolgus CD45, and rhesus CD45. These residues include 405T, 407K, 419Y, 425K, and 505R (numbered with reference to the fragment of hCD45 set forth in SEQ ID NO:113). In addition, these antibodies may interact with residues 481R and/or 509H in human CD45 (numbered with reference to the fragment of hCD45 set forth in SEQ ID NO:113). Accordingly, in some embodiments, provided herein is an antibody, or antigen-binding portion thereof, that binds to human CD45 at an epitope located in the d3 and/or d4 fibronectin-like domains. In some embodiments, provided herein is an antibody, or antigen-binding portion thereof, that binds to CD45 at an epitope of human CD45 located within CD45 fragment 2 (SEQ ID NO:115 and/or CD45 fragment 4 (SEQ ID NO:117). In some embodiments, provided herein is an antibody, or antigen-binding portion thereof, that binds to CD45 at an epitope of human CD45 located within CD45 fragment 1 (SEQ ID NO:114 and/or CD45 fragment 3 (SEQ ID NO:116). In some embodiments, provided herein is an antibody, or antigen-binding portion thereof, that binds to CD45 at an epitope comprising at least one, at least two, at least three, at least four, or least five amino acid residues that are conserved among human CD45, cynomolgus CD45, and/or rhesus CD45. For example, in some embodiments, the antibody, or antigen-binding portion thereof, can bind to at least one, at least two, at least three, at least four, or all five of the following amino acid residues in human CD45: 405T, 407K, 419Y, 425K, and 505R (numbered with reference to the fragment of hCD45 set forth in SEQ ID NO:113). In some embodiments, the antibody, or antigen-binding portion thereof, can bind to one or more, two or more, three or more, four or more, five or more, six or more, or seven of the following amino acid residues in human CD45: 405T, 407K, 419Y, 425K, 481R, and 505R, 509H (numbered with reference to the fragment of hCD45 set forth in SEQ ID NO:113). Also provided herein is an antibody, or antigen-binding portion thereof, that competes with Ab1, Ab2, Ab3, Ab4, Ab5, Ab6, and/or Ab7 for binding to human CD45 (SEQ ID NO:112). In some embodiments, the antibody, or antigen-binding portion thereof, can also compete with Ab1, Ab2, Ab3, Ab4, Ab5, Ab6, and/or Ab7 for binding to cynomolgus CD45 (SEQ ID NO:145), and/or rhesus CD45 (SEQ ID NO:146). In some embodiments, the antibody is a fully human antibody, or antigen-binding portion thereof. In other embodiments, the antibody is a humanized antibody, or antigen-binding portion thereof. In some embodiments, the antibody is a chimeric antibody, or antigen-binding portion thereof. In some embodiments, the antibody is a deimmunized antibody, or antigen-binding portion thereof.

Without wishing to be bound by any theory, it is believed that antibody AbA, described herein, also binds to the d4 fibronectin-like domain of CD45, but does not compete with any of Ab1-Ab7 for binding to CD45. Epitope mapping experiments described herein suggest that AbA binds to the d4 fibronectin-like domain at the opposite face of the molecule, relative to Ab1-Ab7. In particular, this antibody is believed to interact with a fragment of human CD45 set forth in SEQ ID NO:118. Crosslinking studies described herein suggest that AbA can specifically interact with one or more CD45 amino acid residues, which are conserved between human CD45, cynomolgus CD45, and rhesus CD45. These residues include 493Y and 502T (numbered with reference to the fragment of hCD45 set forth in SEQ ID NO:113). In addition, this antibody may interact with residue 486R in human CD45 (numbered with reference to the fragment of hCD45 set forth in SEQ ID NO:113). Accordingly, in some embodiments, provided herein is an antibody, or antigen-binding portion thereof, that binds to human CD45 at an epitope located in the d4 fibronectin-like domain. In some embodiments, provided herein is an antibody, or antigen-binding portion thereof, that binds to CD45 at an epitope of human CD45 located within CD45 fragment 5 (SEQ ID NO:118). In some embodiments, provided herein is an antibody, or antigen-binding portion thereof, that binds to CD45 at an epitope comprising at least one or at least two amino acid residues that are conserved among human CD45, cynomolgus CD45, and/or rhesus CD45. For example, in some embodiments, the antibody, or antigen-binding portion thereof, can bind to one or both of the following amino acid residues in human CD45: 493Y and 502T (numbered with reference to the fragment of hCD45 set forth in SEQ ID NO:113). In some embodiments, the antibody, or antigen-binding portion thereof, can bind to one or more, two or more, or three of the following amino acid residues in human CD45: 486R, 493Y and 502T (numbered with reference to the fragment of hCD45 set forth in SEQ ID NO:113). Also provided herein is an antibody, or antigen-binding portion thereof, that competes with AbA for binding to human CD45 (SEQ ID NO:112). In some embodiments, the antibody, or antigen-binding portion thereof, can also compete with AbA for binding to cynomolgus CD45 (SEQ ID NO:145), and/or rhesus CD45 (SEQ ID NO:146). In some embodiments, the antibody is a fully human antibody, or antigen-binding portion thereof. In other embodiments, the antibody is a humanized antibody, or antigen-binding portion thereof. In some embodiments, the antibody is a chimeric antibody, or antigen-binding portion thereof. In some embodiments, the antibody is a deimmunized antibody, or antigen-binding portion thereof.

In other embodiments, provided herein is an antibody, or antigen-binding portion thereof, that binds to the same epitope of human CD45 as AbB. In some embodiments, the antibody, or antigen-binding portion thereof, cross-reacts with cynomolgus CD45 and/or rhesus CD45. Also provided herein is an antibody, or antigen-binding portion thereof, that competes with AbB for binding to human CD45 (SEQ ID NO:112). In some embodiments, the antibody, or antigen-binding portion thereof, can also compete with AbB for binding to cynomolgus CD45 (SEQ ID NO:145), and/or rhesus CD45 (SEQ ID NO:146). In some embodiments, the antibody is a fully human antibody, or antigen-binding portion thereof. In other embodiments, the antibody is a humanized antibody, or antigen-binding portion thereof. In some embodiments, the antibody is a chimeric antibody, or antigen-binding portion thereof. In some embodiments, the antibody is a deimmunized antibody, or antigen-binding portion thereof.

In other embodiments, provided herein is an antibody, or antigen-binding portion thereof, that binds to the same epitope of human CD45 as AbC. In some embodiments, the antibody, or antigen-binding portion thereof, cross-reacts with cynomolgus CD45 and/or rhesus CD45. Also provided herein is an antibody, or antigen-binding portion thereof, that competes with AbC for binding to human CD45 (SEQ ID NO:112). In some embodiments, the antibody, or antigen-binding portion thereof, can also compete with AbC for binding to cynomolgus CD45 (SEQ ID NO:145), and/or rhesus CD45 (SEQ ID NO:146). In some embodiments, the antibody is a fully human antibody, or antigen-binding portion thereof. In other embodiments, the antibody is a humanized antibody, or antigen-binding portion thereof. In some embodiments, the antibody is a chimeric antibody, or antigen-binding portion thereof. In some embodiments, the antibody is a deimmunized antibody, or antigen-binding portion thereof.

The amino acid sequences of the various binding regions of anti-CD45 antibodies Ab1, Ab2, Ab3, Ab4, Ab5, Ab6, Ab7, AbA, AbB, and AbC are described in Table 3. In various aspect, the disclosure provides an antibody comprising the heavy chain and/or light chain CDR sequences of an antibody described in Table 3. In some aspects, the disclosure provides an antibody comprising the heavy chain variable region and/or the light chain variable region of an antibody described in Table 3. In some aspects, the disclosure provides an antibody comprising the heavy chain and/or the light chain of an antibody described in Table 3. Additional features of the antibodies, and antigen-binding portions thereof, provided herein are described below.

### Ab1

Antibody 1 (Ab1) cross reacts with human CD45, cyno CD45 and rhesus CD45, and can bind the various isoforms of human CD45.

The amino acid sequences for the various binding regions of anti-CD45 Ab1 are described in Table 3. Included in the disclosure are anti-CD45 antibodies based on Ab1, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab1. The heavy chain variable region (VH) amino acid sequence of Ab1 is set forth in SEQ ID NO:1 (see Table 3). The VH CDR domain amino acid sequences of Ab1 are set forth in SEQ ID NO:2 (VH CDR1); SEQ ID NO:3 (VH CDR2), and SEQ ID NO:4 (VH CDR3). The light chain variable region (VL) amino acid sequence of Ab1 is described in SEQ ID NO:5 (see Table 3). The VL CDR domain amino acid sequences of Ab1 are set forth in SEQ ID NO:6 (VL CDR1); SEQ ID NO:7 (VL CDR2), and SEQ ID NO:8 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:2, a CDR2 domain comprising the amino acid sequence as set forth in SEQ ID NO:3, and a CDR3 domain comprising the amino acid sequence as set forth in SEQ ID NO:4; and/or a light chain variable region comprising a CDR1 domain comprising the amino acid sequence as set forth in SEQ ID NO:6, a CDR2 domain comprising the amino acid sequence as set forth in SEQ ID NO:7; and a CDR3 domain comprising the amino acid sequence as set forth in SEQ ID NO:8. In certain embodiments, an anti-CD45 antibody comprises the CDRs described herein (SEQ ID Nos: 2 to 4 and 6 to 8) wherein the CDR comprises a conservative amino acid substitution (or 2, 3, 4, or 5 amino acid substitutions) while retaining the CD45 specificity of the antibody (i.e., specificity similar to Ab1).

In some embodiments, provided herein is an anti-CD45 antibody, or antigen-binding portion thereof, comprising a heavy chain variable region comprising the amino acid sequence as set forth in SEQ ID NO:1, and/or a light chain variable region comprising the amino acid sequence as set forth in SEQ ID NO:5. In certain embodiments, an antibody can comprise a modified heavy chain (HC) variable region comprising an HC variable domain comprising SEQ ID NO:1, or a variant of SEQ ID NO:1, which variant (i) differs from SEQ ID NO:1 in 1, 2, 3, 4 or 5 amino acids substitutions, additions or deletions; (ii) differs from SEQ ID NO:1 in at most 5, 4, 3, 2, or 1 amino acids substitutions, additions or deletions; (iii) differs from SEQ ID NO:1 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:1, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab1), or has an enhanced biological activity relative to that of another Ab1 heavy chain variable region. In certain embodiments, an antibody comprises a modified light chain (LC) variable region comprising an LC variable domain comprising SEQ ID NO:5, or a variant of SEQ ID NO:5, which variant (i) differs from SEQ ID NO:5 in 1, 2, 3, 4 or 5 amino acids substitutions, additions or deletions; (ii) differs from SEQ ID NO:5 in at most 5, 4, 3, 2, or 1 amino acids substitutions, additions or deletions; (iii) differs from SEQ ID NO:5 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:5, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab1), or has an enhanced biological activity relative to that of another Ab1 light chain variable region.

Antibodies comprising the CDR and/or variable region sequences of Ab1 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab1 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab1 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab1 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:9. In certain embodiments, an antibody comprises a modified heavy chain (HC) region comprising an HC domain comprising SEQ ID NO:9, or a variant of SEQ ID NO:9, which variant (i) differs from SEQ ID NO:9 in 1, 2, 3, 4 or 5 amino acids substitutions, additions or deletions; (ii) differs from SEQ ID NO:9 in at most 5, 4, 3, 2, or 1 amino acids substitutions, additions or deletions; (iii) differs from SEQ ID NO:9 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:9, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab1), or has an enhanced biological activity relative to that of another Ab1 heavy chain region.

In certain embodiments, the anti-CD45 antibody, or antigen binding portion thereof, comprises a light chain comprising the amino acid sequence as set forth in SEQ ID NO:10. In certain embodiments, an antibody comprises a modified light chain (LC) region comprising an LC domain comprising SEQ ID NO:10, or a variant of SEQ ID NO:10, which variant (i) differs from SEQ ID NO:10 in 1, 2, 3, 4 or 5 amino acids substitutions, additions or deletions; (ii) differs from SEQ ID NO:10 in at most 5, 4, 3, 2, or 1 amino acids substitutions, additions or deletions; (iii) differs from SEQ ID NO:10 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:10, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab1), or has an enhanced biological activity relative to that of another Ab1 light chain region.

In some embodiments, antibodies comprising the CDR regions and/or variable regions of Ab1 can be incorporated into antibody-drug conjugates, as described herein. In addition, antibodies comprising the CDR regions and/or variable regions of Ab1 can be used in the methods described herein, e.g., for depletion of CD45+ cells in a subject.

### Ab2

Antibody 2 (Ab2) cross reacts with human CD45, cyno CD45 and rhesus CD45, and can bind the various isoforms of human CD45.

The amino acid sequences for the various binding regions of anti-CD45 Ab2 are described in Table 3. Included in the disclosure are anti-CD45 antibodies based on Ab2, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab2. The heavy chain variable region (VH) amino acid sequence of Ab2 is set forth in SEQ ID NO:11 (see Table 3). The VH CDR domain amino acid sequences of Ab2 are set forth in SEQ ID NO:12 (VH CDR1); SEQ ID NO:13 (VH CDR2), and SEQ ID NO:14 (VH CDR3). The light chain variable region (VL) amino acid sequence of Ab2 is described in SEQ ID NO:15 (see Table 3). The VL CDR domain amino acid sequences of Ab2 are set forth in SEQ ID NO:16 (VL CDR1); SEQ ID NO:17 (VL CDR2), and SEQ ID NO:18 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:12, a CDR2 domain comprising the amino acid sequence as set forth in SEQ ID NO:13, and a CDR3 domain comprising the amino acid sequence as set forth in SEQ ID NO:14; and/or a light chain variable region comprising a CDR1 domain comprising the amino acid sequence as set forth in SEQ ID NO:16, a CDR2 domain comprising the amino acid sequence as set forth in SEQ ID NO:17; and a CDR3 domain comprising the amino acid sequence as set forth in SEQ ID NO:18. In certain embodiments, an anti-CD45 antibody comprises the CDRs described herein (SEQ ID NOs: 12 to 14 and 16 to 18) wherein the CDR comprises a conservative amino acid substitution (or 2, 3, 4, or 5 amino acid substitutions) while retaining the CD45 specificity of the antibody (i.e., specificity similar to Ab2).

In some embodiments, provided herein is an anti-CD45 antibody, or antigen-binding portion thereof, comprising a heavy chain variable region comprising the amino acid sequence as set forth in SEQ ID NO:11, and/or a light chain variable region comprising the amino acid sequence as set forth in SEQ ID NO:15. In certain embodiments, an antibody can comprise a modified heavy chain (HC) variable region comprising an HC variable domain comprising SEQ ID NO:11, or a variant of SEQ ID NO:11, which variant (i) differs from SEQ ID NO:11 in 1, 2, 3, 4 or 5 amino acids substitutions, additions or deletions; (ii) differs from SEQ ID NO:11 in at most 5, 4, 3, 2, or 1 amino acids substitutions, additions or deletions; (iii) differs from SEQ ID NO:11 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:11, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab2), or has an enhanced biological activity relative to that of another Ab2 heavy chain variable region. In certain embodiments, an antibody comprises a modified light chain (LC) variable region comprising an LC variable domain comprising SEQ ID NO:15, or a variant of SEQ ID NO:15, which variant (i) differs from SEQ ID NO:15 in 1, 2, 3, 4 or 5 amino acids substitutions, additions or deletions; (ii) differs from SEQ ID NO:15 in at most 5, 4, 3, 2, or 1 amino acids substitutions, additions or deletions; (iii) differs from SEQ ID NO:15 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:15, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab2), or has an enhanced biological activity relative to that of another Ab2 light chain variable region.

Antibodies comprising the CDR and/or variable region sequences of Ab2 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab2 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab2 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab2 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:19. In certain embodiments, an antibody comprises a modified heavy chain (HC) region comprising an HC domain comprising SEQ ID NO:19, or a variant of SEQ ID NO:19, which variant (i) differs from SEQ ID NO:19 in 1, 2, 3, 4 or 5 amino acids substitutions, additions or deletions; (ii) differs from SEQ ID NO:19 in at most 5, 4, 3, 2, or 1 amino acids substitutions, additions or deletions; (iii) differs from SEQ ID NO:19 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:19, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab2), or has an enhanced biological activity relative to that of another Ab2 heavy chain region.

In certain embodiments, the anti-CD45 antibody, or antigen binding portion thereof, comprises a light chain comprising the amino acid sequence as set forth in SEQ ID NO:20. In certain embodiments, an antibody comprises a modified light chain (LC) region comprising an LC domain comprising SEQ ID NO:20, or a variant of SEQ ID NO:20, which variant (i) differs from SEQ ID NO:20 in 1, 2, 3, 4 or 5 amino acids substitutions, additions or deletions; (ii) differs from SEQ ID NO:20 in at most 5, 4, 3, 2, or 1 amino acids substitutions, additions or deletions; (iii) differs from SEQ ID NO:20 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:20, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab2), or has an enhanced biological activity relative to that of another Ab2 light chain region.

In some embodiments, antibodies comprising the CDR regions and/or variable regions of Ab2 can be incorporated into antibody-drug conjugates, as described herein. In addition, antibodies comprising the CDR regions and/or variable regions of Ab2 can be used in the methods described herein, e.g., for depletion of CD45+ cells in a subject.

### Ab3

Antibody 3 (Ab3) cross reacts with human CD45, cyno CD45 and rhesus CD45, and can bind the various isoforms of human CD45.

The amino acid sequences for the various binding regions of anti-CD45 Ab3 are described in Table 3. Included in the disclosure are anti-CD45 antibodies based on Ab3, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab3. The heavy chain variable region (VH) amino acid sequence of Ab3 is set forth in SEQ ID NO:21 (see Table 3). The VH CDR domain amino acid sequences of Ab3 are set forth in SEQ ID NO:22 (VH CDR1); SEQ ID NO:23 (VH CDR2), and SEQ ID NO:24 (VH CDR3). The light chain variable region (VL) amino acid sequence of Ab3 is described in SEQ ID NO:25 (see Table 3). The VL CDR domain amino acid sequences of Ab3 are set forth in SEQ ID NO:26 (VL CDR1); SEQ ID NO:27 (VL CDR2), and SEQ ID NO:28 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:22, a CDR2 domain comprising the amino acid sequence as set forth in SEQ ID NO:23, and a CDR3 domain comprising the amino acid sequence as set forth in SEQ ID NO:24; and/or a light chain variable region comprising a CDR1 domain comprising the amino acid sequence as set forth in SEQ ID NO:26, a CDR2 domain comprising the amino acid sequence as set forth in SEQ ID NO:27; and a CDR3 domain comprising the amino acid sequence as set forth in SEQ ID NO:28. In certain embodiments, an anti-CD45 antibody comprises the CDRs described herein (SEQ ID NOs: 22 to 24 and 26 to 28) wherein the CDR comprises a conservative amino acid substitution (or 2, 3, 4, or 5 amino acid substitutions) while retaining the CD45 specificity of the antibody (i.e., specificity similar to Ab3).

In some embodiments, provided herein is an anti-CD45 antibody, or antigen-binding portion thereof, comprising a heavy chain variable region comprising the amino acid sequence as set forth in SEQ ID NO:21, and/or a light chain variable region comprising the amino acid sequence as set forth in SEQ ID NO:25. In certain embodiments, an antibody can comprise a modified heavy chain (HC) variable region comprising an HC variable domain comprising SEQ ID NO:21, or a variant of SEQ ID NO:21, which variant (i) differs from SEQ ID NO:21 in 1, 2, 3, 4 or 5 amino acids substitutions, additions or deletions; (ii) differs from SEQ ID NO:21 in at most 5, 4, 3, 2, or 1 amino acids substitutions, additions or deletions; (iii) differs from SEQ ID NO:21 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:21, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab3), or has an enhanced biological activity relative to that of another Ab3 heavy chain variable region. In certain embodiments, an antibody comprises a modified light chain (LC) variable region comprising an LC variable domain comprising SEQ ID NO:25, or a variant of SEQ ID NO:25, which variant (i) differs from SEQ ID NO:25 in 1, 2, 3, 4 or 5 amino acids substitutions, additions or deletions; (ii) differs from SEQ ID NO:25 in at most 5, 4, 3, 2, or 1 amino acids substitutions, additions or deletions; (iii) differs from SEQ ID NO:25 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:25, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab3), or has an enhanced biological activity relative to that of another Ab3 light chain variable region.

Antibodies comprising the CDR and/or variable region sequences of Ab3 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab3 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab3 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab3 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:29. In certain embodiments, an antibody comprises a modified heavy chain (HC) region comprising an HC domain comprising SEQ ID NO:29, or a variant of SEQ ID NO:29, which variant (i) differs from SEQ ID NO:29 in 1, 2, 3, 4 or 5 amino acids substitutions, additions or deletions; (ii) differs from SEQ ID NO:29 in at most 5, 4, 3, 2, or 1 amino acids substitutions, additions or deletions; (iii) differs from SEQ ID NO:29 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:29, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab3), or has an enhanced biological activity relative to that of another Ab3 heavy chain region.

In certain embodiments, the anti-CD45 antibody, or antigen binding portion thereof, comprises a light chain comprising the amino acid sequence as set forth in SEQ ID NO:30. In certain embodiments, an antibody comprises a modified light chain (LC) region comprising an LC domain comprising SEQ ID NO:30, or a variant of SEQ ID NO:30, which variant (i) differs from SEQ ID NO:30 in 1, 2, 3, 4 or 5 amino acids substitutions, additions or deletions; (ii) differs from SEQ ID NO:30 in at most 5, 4, 3, 2, or 1 amino acids substitutions, additions or deletions; (iii) differs from SEQ ID NO:30 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:30, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab3), or has an enhanced biological activity relative to that of another Ab3 light chain region.

In some embodiments, antibodies comprising the CDR regions and/or variable regions of Ab3 can be incorporated into antibody-drug conjugates, as described herein. In addition, antibodies comprising the CDR regions and/or variable regions of Ab3 can be used in the methods described herein, e.g., for depletion of CD45+ cells in a subject.

### Ab4

Antibody 4 (Ab4) cross reacts with human CD45, cyno CD45 and rhesus CD45, and can bind the various isoforms of human CD45.

The amino acid sequences for the various binding regions of anti-CD45 Ab4 are described in Table 3. Included in the disclosure are anti-CD45 antibodies based on Ab4, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab4. The heavy chain variable region (VH) amino acid sequence of Ab4 is set forth in SEQ ID NO:31 (see Table 3). The VH CDR domain amino acid sequences of Ab4 are set forth in SEQ ID NO:32 (VH CDR1); SEQ ID NO:33 (VH CDR2), and SEQ ID NO:34 (VH CDR3). The light chain variable region (VL) amino acid sequence of Ab4 is described in SEQ ID NO:35 (see Table 3). The VL CDR domain amino acid sequences of Ab4 are set forth in SEQ ID NO:36 (VL CDR1); SEQ ID NO:37 (VL CDR2), and SEQ ID NO:38 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:32, a CDR2 domain comprising the amino acid sequence as set forth in SEQ ID NO:33, and a CDR3 domain comprising the amino acid sequence as set forth in SEQ ID NO:34; and/or a light chain variable region comprising a CDR1 domain comprising the amino acid sequence as set forth in SEQ ID NO:36, a CDR2 domain comprising the amino acid sequence as set forth in SEQ ID NO:37; and a CDR3 domain comprising the amino acid sequence as set forth in SEQ ID NO:38. In certain embodiments, an anti-CD45 antibody comprises the CDRs described herein (SEQ ID NOs: 32 to 34 and 36 to 38) wherein the CDR comprises a conservative amino acid substitution (or 2, 3, 4, or 5 amino acid substitutions) while retaining the CD45 specificity of the antibody (i.e., specificity similar to Ab4).

In some embodiments, provided herein is an anti-CD45 antibody, or antigen-binding portion thereof, comprising a heavy chain variable region comprising the amino acid sequence as set forth in SEQ ID NO:31, and/or a light chain variable region comprising the amino acid sequence as set forth in SEQ ID NO:35. In certain embodiments, an antibody can comprise a modified heavy chain (HC) variable region comprising an HC variable domain comprising SEQ ID NO:31, or a variant of SEQ ID NO:31, which variant (i) differs from SEQ ID NO:31 in 1, 2, 3, 4 or 5 amino acids substitutions, additions or deletions; (ii) differs from SEQ ID NO:31 in at most 5, 4, 3, 2, or 1 amino acids substitutions, additions or deletions; (iii) differs from SEQ ID NO:31 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:31, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab4), or has an enhanced biological activity relative to that of another Ab4 heavy chain variable region. In certain embodiments, an antibody comprises a modified light chain (LC) variable region comprising an LC variable domain comprising SEQ ID NO:35, or a variant of SEQ ID NO:35, which variant (i) differs from SEQ ID NO:35 in 1, 2, 3, 4 or 5 amino acids substitutions, additions or deletions; (ii) differs from SEQ ID NO:35 in at most 5, 4, 3, 2, or 1 amino acids substitutions, additions or deletions; (iii) differs from SEQ ID NO:35 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:35, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab4), or has an enhanced biological activity relative to that of another Ab4 light chain variable region.

Antibodies comprising the CDR and/or variable region sequences of Ab4 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab4 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab4 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab4 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:39. In certain embodiments, an antibody comprises a modified heavy chain (HC) region comprising an HC domain comprising SEQ ID NO:39, or a variant of SEQ ID NO:39, which variant (i) differs from SEQ ID NO:39 in 1, 2, 3, 4 or 5 amino acids substitutions, additions or deletions; (ii) differs from SEQ ID NO:39 in at most 5, 4, 3, 2, or 1 amino acids substitutions, additions or deletions; (iii) differs from SEQ ID NO:39 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:39, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab4), or has an enhanced biological activity relative to that of another Ab4 heavy chain region.

In certain embodiments, the anti-CD45 antibody, or antigen binding portion thereof, comprises a light chain comprising the amino acid sequence as set forth in SEQ ID NO:40. In certain embodiments, an antibody comprises a modified light chain (LC) region comprising an LC domain comprising SEQ ID NO:40, or a variant of SEQ ID NO:40, which variant (i) differs from SEQ ID NO:40 in 1, 2, 3, 4 or 5 amino acids substitutions, additions or deletions; (ii) differs from SEQ ID NO:40 in at most 5, 4, 3, 2, or 1 amino acids substitutions, additions or deletions; (iii) differs from SEQ ID NO:40 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:40, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab4), or has an enhanced biological activity relative to that of another Ab4 light chain region.

In some embodiments, antibodies comprising the CDR regions and/or variable regions of Ab4 can be incorporated into antibody-drug conjugates, as described herein. In addition, antibodies comprising the CDR regions and/or variable regions of Ab4 can be used in the methods described herein, e.g., for depletion of CD45+ cells in a subject.

### Ab5

Antibody 5 (Ab5) cross reacts with human CD45, cyno CD45 and rhesus CD45, and can bind the various isoforms of human CD45.

The amino acid sequences for the various binding regions of anti-CD45 Ab5 are described in Table 3. Included in the disclosure are anti-CD45 antibodies based on Ab5, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab5. The heavy chain variable region (VH) amino acid sequence of Ab5 is set forth in SEQ ID NO:41 (see Table 3). The VH CDR domain amino acid sequences of Ab5 are set forth in SEQ ID NO:42 (VH CDR1); SEQ ID NO:43 (VH CDR2), and SEQ ID NO:44 (VH CDR3). The light chain variable region (VL) amino acid sequence of Ab5 is described in SEQ ID NO:45 (see Table 3). The VL CDR domain amino acid sequences of Ab5 are set forth in SEQ ID NO:46 (VL CDR1); SEQ ID NO:47 (VL CDR2), and SEQ ID NO:48 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:42, a CDR2 domain comprising the amino acid sequence as set forth in SEQ ID NO:43, and a CDR3 domain comprising the amino acid sequence as set forth in SEQ ID NO:44; and/or a light chain variable region comprising a CDR1 domain comprising the amino acid sequence as set forth in SEQ ID NO:46, a CDR2 domain comprising the amino acid sequence as set forth in SEQ ID NO:47; and a CDR3 domain comprising the amino acid sequence as set forth in SEQ ID NO:48. In certain embodiments, an anti-CD45 antibody comprises the CDRs described herein (SEQ ID NOs: 42 to 44 and 46 to 48) wherein the CDR comprises a conservative amino acid substitution (or 2, 3, 4, or 5 amino acid substitutions) while retaining the CD45 specificity of the antibody (i.e., specificity similar to Ab5).

In some embodiments, provided herein is an anti-CD45 antibody, or antigen-binding portion thereof, comprising a heavy chain variable region comprising the amino acid sequence as set forth in SEQ ID NO:41, and/or a light chain variable region comprising the amino acid sequence as set forth in SEQ ID NO:45. In certain embodiments, an antibody can comprise a modified heavy chain (HC) variable region comprising an HC variable domain comprising SEQ ID NO:41, or a variant of SEQ ID NO:41, which variant (i) differs from SEQ ID NO:41 in 1, 2, 3, 4 or 5 amino acids substitutions, additions or deletions; (ii) differs from SEQ ID NO:41 in at most 5, 4, 3, 2, or 1 amino acids substitutions, additions or deletions; (iii) differs from SEQ ID NO:41 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:41, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab5), or has an enhanced biological activity relative to that of another Ab5 heavy chain variable region. In certain embodiments, an antibody comprises a modified light chain (LC) variable region comprising an LC variable domain comprising SEQ ID NO:45, or a variant of SEQ ID NO:45, which variant (i) differs from SEQ ID NO:45 in 1, 2, 3, 4 or 5 amino acids substitutions, additions or deletions; (ii) differs from SEQ ID NO:45 in at most 5, 4, 3, 2, or 1 amino acids substitutions, additions or deletions; (iii) differs from SEQ ID NO:45 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:45, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab5), or has an enhanced biological activity relative to that of another Ab5 light chain variable region.

Antibodies comprising the CDR and/or variable region sequences of Ab5 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab5 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab5 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab5 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:49. In certain embodiments, an antibody comprises a modified heavy chain (HC) region comprising an HC domain comprising SEQ ID NO:49, or a variant of SEQ ID NO:9, which variant (i) differs from SEQ ID NO:49 in 1, 2, 3, 4 or 5 amino acids substitutions, additions or deletions; (ii) differs from SEQ ID NO:49 in at most 5, 4, 3, 2, or 1 amino acids substitutions, additions or deletions; (iii) differs from SEQ ID NO:49 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:49, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab5), or has an enhanced biological activity relative to that of another Ab5 heavy chain region.

In certain embodiments, the anti-CD45 antibody, or antigen binding portion thereof, comprises a light chain comprising the amino acid sequence as set forth in SEQ ID NO:50. In certain embodiments, an antibody comprises a modified light chain (LC) region comprising an LC domain comprising SEQ ID NO:50, or a variant of SEQ ID NO:50, which variant (i) differs from SEQ ID NO:50 in 1, 2, 3, 4 or 5 amino acids substitutions, additions or deletions; (ii) differs from SEQ ID NO:50 in at most 5, 4, 3, 2, or 1 amino acids substitutions, additions or deletions; (iii) differs from SEQ ID NO:50 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:50, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab5), or has an enhanced biological activity relative to that of another Ab5 light chain region.

In some embodiments, antibodies comprising the CDR regions and/or variable regions of Ab5 can be incorporated into antibody-drug conjugates, as described herein. In addition, antibodies comprising the CDR regions and/or variable regions of Ab5 can be used in the methods described herein, e.g., for depletion of CD45+ cells in a subject.

### Ab6

Antibody 6 (Ab6) cross reacts with human CD45, cyno CD45 and rhesus CD45, and can bind the various isoforms of human CD45.

The amino acid sequences for the various binding regions of anti-CD45 Ab6 are described in Table 3. Included in the disclosure are anti-CD45 antibodies based on Ab6, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab6. The heavy chain variable region (VH) amino acid sequence of Ab6 is set forth in SEQ ID NO:51 (see Table 3). The VH CDR domain amino acid sequences of Ab6 are set forth in SEQ ID NO:52 (VH CDR1); SEQ ID NO:53 (VH CDR2), and SEQ ID NO:54 (VH CDR3). The light chain variable region (VL) amino acid sequence of Ab6 is described in SEQ ID NO:55 (see Table 3). The VL CDR domain amino acid sequences of Ab6 are set forth in SEQ ID NO:56 (VL CDR1); SEQ ID NO:57 (VL CDR2), and SEQ ID NO:58 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:52, a CDR2 domain comprising the amino acid sequence as set forth in SEQ ID NO:53, and a CDR3 domain comprising the amino acid sequence as set forth in SEQ ID NO:54; and/or a light chain variable region comprising a CDR1 domain comprising the amino acid sequence as set forth in SEQ ID NO:56, a CDR2 domain comprising the amino acid sequence as set forth in SEQ ID NO:57; and a CDR3 domain comprising the amino acid sequence as set forth in SEQ ID NO:58. In certain embodiments, an anti-CD45 antibody comprises the CDRs described herein (SEQ ID NOs: 52 to 54 and 56 to 58) wherein the CDR comprises a conservative amino acid substitution (or 2, 3, 4, or 5 amino acid substitutions) while retaining the CD45 specificity of the antibody (i.e., specificity similar to Ab6).

In some embodiments, provided herein is an anti-CD45 antibody, or antigen-binding portion thereof, comprising a heavy chain variable region comprising the amino acid sequence as set forth in SEQ ID NO:51, and/or a light chain variable region comprising the amino acid sequence as set forth in SEQ ID NO:55. In certain embodiments, an antibody can comprise a modified heavy chain (HC) variable region comprising an HC variable domain comprising SEQ ID NO:51, or a variant of SEQ ID NO:51, which variant (i) differs from SEQ ID NO:51 in 1, 2, 3, 4 or 5 amino acids substitutions, additions or deletions; (ii) differs from SEQ ID NO:51 in at most 5, 4, 3, 2, or 1 amino acids substitutions, additions or deletions; (iii) differs from SEQ ID NO:51 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:51, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab6), or has an enhanced biological activity relative to that of another Ab6 heavy chain variable region. In certain embodiments, an antibody comprises a modified light chain (LC) variable region comprising an LC variable domain comprising SEQ ID NO:55, or a variant of SEQ ID NO:55, which variant (i) differs from SEQ ID NO:55 in 1, 2, 3, 4 or 5 amino acids substitutions, additions or deletions; (ii) differs from SEQ ID NO:55 in at most 5, 4, 3, 2, or 1 amino acids substitutions, additions or deletions; (iii) differs from SEQ ID NO:55 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:55, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab6), or has an enhanced biological activity relative to that of another Ab6 light chain variable region.

Antibodies comprising the CDR and/or variable region sequences of Ab6 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab6 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab6 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab6 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:59. In certain embodiments, an antibody comprises a modified heavy chain (HC) region comprising an HC domain comprising SEQ ID NO:59, or a variant of SEQ ID NO:59, which variant (i) differs from SEQ ID NO:59 in 1, 2, 3, 4 or 5 amino acids substitutions, additions or deletions; (ii) differs from SEQ ID NO:59 in at most 5, 4, 3, 2, or 1 amino acids substitutions, additions or deletions; (iii) differs from SEQ ID NO:59 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:59, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab6), or has an enhanced biological activity relative to that of another Ab6 heavy chain region.

In certain embodiments, the anti-CD45 antibody, or antigen binding portion thereof, comprises a light chain comprising the amino acid sequence as set forth in SEQ ID NO:60. In certain embodiments, an antibody comprises a modified light chain (LC) region comprising an LC domain comprising SEQ ID NO:60, or a variant of SEQ ID NO:60, which variant (i) differs from SEQ ID NO:60 in 1, 2, 3, 4 or 5 amino acids substitutions, additions or deletions; (ii) differs from SEQ ID NO:60 in at most 5, 4, 3, 2, or 1 amino acids substitutions, additions or deletions; (iii) differs from SEQ ID NO:60 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:60, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab6), or has an enhanced biological activity relative to that of another Ab6 light chain region.

In some embodiments, antibodies comprising the CDR regions and/or variable regions of Ab6 can be incorporated into antibody-drug conjugates, as described herein. In addition, antibodies comprising the CDR regions and/or variable regions of Ab6 can be used in the methods described herein, e.g., for depletion of CD45+ cells in a subject.

### Ab7

Antibody 7 (Ab7) cross reacts with human CD45, cyno CD45 and rhesus CD45, and can bind the various isoforms of human CD45.

The amino acid sequences for the various binding regions of anti-CD45 Ab7 are described in Table 3. Included in the disclosure are anti-CD45 antibodies based on Ab7, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of Ab7. The heavy chain variable region (VH) amino acid sequence of Ab7 is set forth in SEQ ID NO:61 (see Table 3). The VH CDR domain amino acid sequences of Ab7 are set forth in SEQ ID NO:62 (VH CDR1); SEQ ID NO:63 (VH CDR2), and SEQ ID NO:64 (VH CDR3). The light chain variable region (VL) amino acid sequence of Ab7 is described in SEQ ID NO:65 (see Table 3). The VL CDR domain amino acid sequences of Ab7 are set forth in SEQ ID NO:66 (VL CDR1); SEQ ID NO:67 (VL CDR2), and SEQ ID NO:68 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:62, a CDR2 domain comprising the amino acid sequence as set forth in SEQ ID NO:63, and a CDR3 domain comprising the amino acid sequence as set forth in SEQ ID NO:64; and/or a light chain variable region comprising a CDR1 domain comprising the amino acid sequence as set forth in SEQ ID NO:66, a CDR2 domain comprising the amino acid sequence as set forth in SEQ ID NO:67; and a CDR3 domain comprising the amino acid sequence as set forth in SEQ ID NO:68. In certain embodiments, an anti-CD45 antibody comprises the CDRs described herein (SEQ ID NOs: 62 to 64 and 66 to 68) wherein the CDR comprises a conservative amino acid substitution (or 2, 3, 4, or 5 amino acid substitutions) while retaining the CD45 specificity of the antibody (i.e., specificity similar to Ab7).

In some embodiments, provided herein is an anti-CD45 antibody, or antigen-binding portion thereof, comprising a heavy chain variable region comprising the amino acid sequence as set forth in SEQ ID NO:61, and/or a light chain variable region comprising the amino acid sequence as set forth in SEQ ID NO:65. In certain embodiments, an antibody can comprise a modified heavy chain (HC) variable region comprising an HC variable domain comprising SEQ ID NO:61, or a variant of SEQ ID NO:61, which variant (i) differs from SEQ ID NO:61 in 1, 2, 3, 4 or 5 amino acids substitutions, additions or deletions; (ii) differs from SEQ ID NO:61 in at most 5, 4, 3, 2, or 1 amino acids substitutions, additions or deletions; (iii) differs from SEQ ID NO:61 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:61, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to Ab7), or has an enhanced biological activity relative to that of another Ab7 heavy chain variable region. In certain embodiments, an antibody comprises a modified light chain (LC) variable region comprising an LC variable domain comprising SEQ ID NO:65, or a variant of SEQ ID NO:65, which variant (i) differs from SEQ ID NO:65 in 1, 2, 3, 4 or 5 amino acids substitutions, additions or deletions; (ii) differs from SEQ ID NO:65 in at most 5, 4, 3, 2, or 1 amino acids substitutions, additions or deletions; (iii) differs from SEQ ID NO:65 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:65, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to Ab7), or has an enhanced biological activity relative to that of another Ab7 light chain variable region.

Antibodies comprising the CDR and/or variable region sequences of Ab7 can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of Ab7 can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of Ab7 can further comprise an IgG1 heavy chain constant region containing one or more of the following substitutions: L234A, L235A, D265C, and H435A (EU index according to Kabat). In some embodiments, an antibody comprising the CDR and/or variable region sequences of Ab7 can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:69. In certain embodiments, an antibody comprises a modified heavy chain (HC) region comprising an HC domain comprising SEQ ID NO:69, or a variant of SEQ ID NO:69, which variant (i) differs from SEQ ID NO:9 in 1, 2, 3, 4 or 5 amino acids substitutions, additions or deletions; (ii) differs from SEQ ID NO:69 in at most 5, 4, 3, 2, or 1 amino acids substitutions, additions or deletions; (iii) differs from SEQ ID NO:69 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:69, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to Ab7), or has an enhanced biological activity relative to that of another Ab7 heavy chain region.

In certain embodiments, the anti-CD45 antibody, or antigen binding portion thereof, comprises a light chain comprising the amino acid sequence as set forth in SEQ ID NO:70. In certain embodiments, an antibody comprises a modified light chain (LC) region comprising an LC domain comprising SEQ ID NO:70, or a variant of SEQ ID NO:70, which variant (i) differs from SEQ ID NO:70 in 1, 2, 3, 4 or 5 amino acids substitutions, additions or deletions; (ii) differs from SEQ ID NO:70 in at most 5, 4, 3, 2, or 1 amino acids substitutions, additions or deletions; (iii) differs from SEQ ID NO:70 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:70, wherein in any of (i)-(iv), an amino acid substitution may be a conservative amino acid substitution or a non-conservative amino acid substitution; and wherein the modified light chain region retains the CD45 specificity of the antibody (i.e., specificity similar to Ab7), or has an enhanced biological activity relative to that of another Ab7 light chain region.

In some embodiments, antibodies comprising the CDR regions and/or variable regions of Ab7 can be incorporated into antibody-drug conjugates, as described herein. In addition, antibodies comprising the CDR regions and/or variable regions of Ab7 can be used in the methods described herein, e.g., for depletion of CD45+ cells in a subject.

### AbA

Antibody A (AbA) cross reacts with human CD45, cyno CD45 and rhesus CD45, and can bind the various isoforms of human CD45.

The amino acid sequences for the various binding regions of anti-CD45 AbA are described in Table 3. Included in the disclosure are anti-CD45 antibodies based on AbA, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of AbA. The heavy chain variable region (VH) amino acid sequence of AbA is set forth in SEQ ID NO:71 (see Table 3). The VH CDR domain amino acid sequences of AbA are set forth in SEQ ID NO:72 (VH CDR1); SEQ ID NO:73 (VH CDR2), and SEQ ID NO:74 (VH CDR3). The light chain variable region (VL) amino acid sequence of AbA is described in SEQ ID NO:75 (see Table 3). The VL CDR domain amino acid sequences of AbA are set forth in SEQ ID NO:76 (VL CDR1); SEQ ID NO:77 (VL CDR2), and SEQ ID NO:78 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:72, a CDR2 domain comprising the amino acid sequence as set forth in SEQ ID NO:73, and a CDR3 domain comprising the amino acid sequence as set forth in SEQ ID NO:74; and/or a light chain variable region comprising a CDR1 domain comprising the amino acid sequence as set forth in SEQ ID NO:76, a CDR2 domain comprising the amino acid sequence as set forth in SEQ ID NO:77; and a CDR3 domain comprising the amino acid sequence as set forth in SEQ ID NO:78. In certain embodiments, an anti-CD45 antibody comprises the CDRs described herein (SEQ ID NOs: 72 to 74 and 76 to 78) wherein the CDR comprises a conservative amino acid substitution (or 2, 3, 4, or 5 amino acid substitutions) while retaining the CD45 specificity of the antibody (i.e., specificity similar to AbA).

In some embodiments, provided herein is an anti-CD45 antibody, or antigen-binding portion thereof, comprising a heavy chain variable region comprising the amino acid sequence as set forth in SEQ ID NO:71, and/or a light chain variable region comprising the amino acid sequence as set forth in SEQ ID NO:75. In certain embodiments, an antibody can comprise a modified heavy chain (HC) variable region comprising an HC variable domain comprising SEQ ID NO:71, or a variant of SEQ ID NO:71, which variant (i) differs from SEQ ID NO:71 in 1, 2, 3, 4 or 5 amino acids substitutions, additions or deletions; (ii) differs from SEQ ID NO:71 in at most 5, 4, 3, 2, or 1 amino acids substitutions, additions or deletions; (iii) differs from SEQ ID NO:71 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:71, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to AbA), or has an enhanced biological activity relative to that of another AbA heavy chain variable region. In certain embodiments, an antibody comprises a modified light chain (LC) variable region comprising an LC variable domain comprising SEQ ID NO:75, or a variant of SEQ ID NO:75, which variant (i) differs from SEQ ID NO:75 in 1, 2, 3, 4 or 5 amino acids substitutions, additions or deletions; (ii) differs from SEQ ID NO:75 in at most 5, 4, 3, 2, or 1 amino acids substitutions, additions or deletions; (iii) differs from SEQ ID NO:75 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:75, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to AbA), or has an enhanced biological activity relative to that of another AbA light chain variable region.

Antibodies comprising the CDR and/or variable region sequences of AbA can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of AbA can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of AbA can further comprise an IgG1 heavy chain constant region containing one or more of the following substitutions: L234A, L235A, D265C, and H435A (EU index according to Kabat). In some embodiments, an antibody comprising the CDR and/or variable region sequences of AbA can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:79. In certain embodiments, an antibody comprises a modified heavy chain (HC) region comprising an HC domain comprising SEQ ID NO:79, or a variant of SEQ ID NO:79, which variant (i) differs from SEQ ID NO:79 in 1, 2, 3, 4 or 5 amino acids substitutions, additions or deletions; (ii) differs from SEQ ID NO:79 in at most 5, 4, 3, 2, or 1 amino acids substitutions, additions or deletions; (iii) differs from SEQ ID NO:79 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:79, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to AbA), or has an enhanced biological activity relative to that of another AbA heavy chain region.

In certain embodiments, the anti-CD45 antibody, or antigen binding portion thereof, comprises a light chain comprising the amino acid sequence as set forth in SEQ ID NO:80. In certain embodiments, an antibody comprises a modified light chain (LC) region comprising an LC domain comprising SEQ ID NO:80, or a variant of SEQ ID NO:80, which variant (i) differs from SEQ ID NO:80 in 1, 2, 3, 4 or 5 amino acids substitutions, additions or deletions; (ii) differs from SEQ ID NO:80 in at most 5, 4, 3, 2, or 1 amino acids substitutions, additions or deletions; (iii) differs from SEQ ID NO:80 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:80, wherein in any of (i)-(iv), an amino acid substitution may be a conservative amino acid substitution or a non-conservative amino acid substitution; and wherein the modified light chain region retains the CD45 specificity of the antibody (i.e., specificity similar to AbA), or has an enhanced biological activity relative to that of another AbA light chain region.

In some embodiments, antibodies comprising the CDR regions and/or variable regions of AbA can be incorporated into antibody-drug conjugates, as described herein. In addition, antibodies comprising the CDR regions and/or variable regions of AbA can be used in the methods described herein, e.g., for depletion of CD45+ cells in a subject.

### AbB

Antibody B (AbB) cross reacts with human CD45, cyno CD45 and rhesus CD45, and can bind the various isoforms of human CD45.

The amino acid sequences for the various binding regions of anti-CD45 AbB are described in Table 3. Included in the disclosure are anti-CD45 antibodies based on AbB, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of AbB. The heavy chain variable region (VH) amino acid sequence of AbB is set forth in SEQ ID NO:81 (see Table 3). The VH CDR domain amino acid sequences of AbB are set forth in SEQ ID NO:82 (VH CDR1); SEQ ID NO:83 (VH CDR2), and SEQ ID NO:84 (VH CDR3). The light chain variable region (VL) amino acid sequence of AbB is described in SEQ ID NO:85 (see Table 3). The VL CDR domain amino acid sequences of AbB are set forth in SEQ ID NO:86 (VL CDR1); SEQ ID NO:87 (VL CDR2), and SEQ ID NO:88 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:82, a CDR2 domain comprising the amino acid sequence as set forth in SEQ ID NO:83, and a CDR3 domain comprising the amino acid sequence as set forth in SEQ ID NO:84; and/or a light chain variable region comprising a CDR1 domain comprising the amino acid sequence as set forth in SEQ ID NO:86, a CDR2 domain comprising the amino acid sequence as set forth in SEQ ID NO:87; and a CDR3 domain comprising the amino acid sequence as set forth in SEQ ID NO:88. In certain embodiments, an anti-CD45 antibody comprises the CDRs described herein (SEQ ID NOs: 82 to 84 and 86 to 88) wherein the CDR comprises a conservative amino acid substitution (or 2, 3, 4, or 5 amino acid substitutions) while retaining the CD45 specificity of the antibody (i.e., specificity similar to AbB).

In some embodiments, provided herein is an anti-CD45 antibody, or antigen-binding portion thereof, comprising a heavy chain variable region comprising the amino acid sequence as set forth in SEQ ID NO:81, and/or a light chain variable region comprising the amino acid sequence as set forth in SEQ ID NO:85. In certain embodiments, an antibody can comprise a modified heavy chain (HC) variable region comprising an HC variable domain comprising SEQ ID NO:81, or a variant of SEQ ID NO:81, which variant (i) differs from SEQ ID NO:81 in 1, 2, 3, 4 or 5 amino acids substitutions, additions or deletions; (ii) differs from SEQ ID NO:81 in at most 5, 4, 3, 2, or 1 amino acids substitutions, additions or deletions; (iii) differs from SEQ ID NO:81 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:81, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to AbB), or has an enhanced biological activity relative to that of another AbB heavy chain variable region. In certain embodiments, an antibody comprises a modified light chain (LC) variable region comprising an LC variable domain comprising SEQ ID NO:85, or a variant of SEQ ID NO:85, which variant (i) differs from SEQ ID NO:85 in 1, 2, 3, 4 or 5 amino acids substitutions, additions or deletions; (ii) differs from SEQ ID NO:85 in at most 5, 4, 3, 2, or 1 amino acids substitutions, additions or deletions; (iii) differs from SEQ ID NO:85 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:85, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to AbB), or has an enhanced biological activity relative to that of another AbB light chain variable region.

Antibodies comprising the CDR and/or variable region sequences of AbB can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of AbB can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of AbB can further comprise an IgG1 heavy chain constant region containing one or more of the following substitutions: L234A, L235A, D265C, and H435A (EU index according to Kabat). In some embodiments, an antibody comprising the CDR and/or variable region sequences of AbB can further comprise a heavy chain constant region set forth in SEQ ID NO:102, SEQ ID NO:103, SEQ ID NO:104, SEQ ID NO:105 or SEQ ID NO:106. In some embodiments the antibody comprises a light chain constant region set forth in SEQ ID NO:101.

In certain embodiments, the anti-CD45 antibody, or antigen binding portion thereof, comprises a heavy chain comprising the amino acid sequence as set forth in SEQ ID NO:89. In certain embodiments, an antibody comprises a modified heavy chain (HC) region comprising an HC domain comprising SEQ ID NO:89, or a variant of SEQ ID NO:89, which variant (i) differs from SEQ ID NO:89 in 1, 2, 3, 4 or 5 amino acids substitutions, additions or deletions; (ii) differs from SEQ ID NO:89 in at most 5, 4, 3, 2, or 1 amino acids substitutions, additions or deletions; (iii) differs from SEQ ID NO:89 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:89, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to AbB), or has an enhanced biological activity relative to that of another AbB heavy chain region.

In certain embodiments, the anti-CD45 antibody, or antigen binding portion thereof, comprises a light chain comprising the amino acid sequence as set forth in SEQ ID NO:90. In certain embodiments, an antibody comprises a modified light chain (LC) region comprising an LC domain comprising SEQ ID NO:90, or a variant of SEQ ID NO:90, which variant (i) differs from SEQ ID NO:90 in 1, 2, 3, 4 or 5 amino acids substitutions, additions or deletions; (ii) differs from SEQ ID NO:90 in at most 5, 4, 3, 2, or 1 amino acids substitutions, additions or deletions; (iii) differs from SEQ ID NO:90 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:90, wherein in any of (i)-(iv), an amino acid substitution may be a conservative amino acid substitution or a non-conservative amino acid substitution; and wherein the modified light chain region retains the CD45 specificity of the antibody (i.e., specificity similar to AbB), or has an enhanced biological activity relative to that of another AbB light chain region.

In some embodiments, antibodies comprising the CDR regions and/or variable regions of AbB can be incorporated into antibody-drug conjugates, as described herein. In addition, antibodies comprising the CDR regions and/or variable regions of AbB can be used in the methods described herein, e.g., for depletion of CD45+ cells in a subject.

### AbC

Antibody C (AbC) cross reacts with human CD45, cyno CD45 and rhesus CD45, and can bind the various isoforms of human CD45.

The amino acid sequences for the various binding regions of anti-CD45 AbC are described in Table 3. Included in the disclosure are anti-CD45 antibodies based on AbC, e.g., that comprise the CDRs as set forth in Table 3.

In one embodiment, an anti-CD45 antibody, or antigen-binding fragment thereof, comprising antigen binding regions, e.g., CDRs and/or variable regions, corresponds to those of AbC. The heavy chain variable region (VH) amino acid sequence of AbC is set forth in SEQ ID NO:91 (see Table 3). The VH CDR domain amino acid sequences of AbC are set forth in SEQ ID NO:92 (VH CDR1); SEQ ID NO:93 (VH CDR2), and SEQ ID NO:94 (VH CDR3). The light chain variable region (VL) amino acid sequence of AbC is described in SEQ ID NO:95 (see Table 3). The VL CDR domain amino acid sequences of AbC are set forth in SEQ ID NO:96 (VL CDR1); SEQ ID NO:97 (VL CDR2), and SEQ ID NO:98 (VL CDR3).

Accordingly, in some embodiments, provided herein is an anti-CD45 antibody, or antigen-binding portion thereof, comprising a heavy chain variable region comprising a CDR1 domain comprising the amino acid sequence as set forth in SEQ ID NO:92, a CDR2 domain comprising the amino acid sequence as set forth in SEQ ID NO:93, and a CDR3 domain comprising the amino acid sequence as set forth in SEQ ID NO:94; and/or a light chain variable region comprising a CDR1 domain comprising the amino acid sequence as set forth in SEQ ID NO:96, a CDR2 domain comprising the amino acid sequence as set forth in SEQ ID NO:97; and a CDR3 domain comprising the amino acid sequence as set forth in SEQ ID NO:98. In certain embodiments, an anti-CD45 antibody comprises the CDRs described herein (SEQ ID NOs: 92 to 94 and 96 to 98) wherein the CDR comprises a conservative amino acid substitution (or 2, 3, 4, or 5 amino acid substitutions) while retaining the CD45 specificity of the antibody (i.e., specificity similar to AbC).

In some embodiments, provided herein is an anti-CD45 antibody, or antigen-binding portion thereof, comprising a heavy chain variable region comprising the amino acid sequence as set forth in SEQ ID NO:991, and/or a light chain variable region comprising the amino acid sequence as set forth in SEQ ID NO:95. In certain embodiments, an antibody can comprise a modified heavy chain (HC) variable region comprising an HC variable domain comprising SEQ ID NO:91, or a variant of SEQ ID NO:91, which variant (i) differs from SEQ ID NO:91 in 1, 2, 3, 4 or 5 amino acids substitutions, additions or deletions; (ii) differs from SEQ ID NO:91 in at most 5, 4, 3, 2, or 1 amino acids substitutions, additions or deletions; (iii) differs from SEQ ID NO:91 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:91, wherein in any of (i)-(iv), an amino acid substitution may be a conservative amino acid substitution or a non-conservative amino acid substitution; and wherein the modified heavy chain variable region retains the CD45 specificity of the antibody (i.e., specificity similar to AbC), or has an enhanced biological activity relative to that of another AbC heavy chain variable region. In certain embodiments, an antibody comprises a modified light chain (LC) variable region comprising an LC variable domain comprising SEQ ID NO:95, or a variant of SEQ ID NO:95, which variant (i) differs from SEQ ID NO:95 in 1, 2, 3, 4 or 5 amino acids substitutions, additions or deletions; (ii) differs from SEQ ID NO:95 in at most 5, 4, 3, 2, or 1 amino acids substitutions, additions or deletions; (iii) differs from SEQ ID NO:95 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:95, wherein in any of (i)-(iv), an amino acid substitution may be a conservative amino acid substitution or a non-conservative amino acid substitution; and wherein the modified light chain variable region retains the CD45 specificity of the antibody (i.e., specificity similar to AbC), or has an enhanced biological activity relative to that of another AbC light chain variable region.

Antibodies comprising the CDR and/or variable region sequences of AbC can be in the form of full-length antibodies, bispecific antibodies, dual variable domain antibodies, multiple chain or single chain antibodies, and/or binding fragments that specifically bind human CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-45 antibody is an IgG (e.g. IgG1, IgG2, IgG3 or IgG4).

In some embodiments, an antibody comprising the CDR and/or variable region sequences of AbC can further comprise a heavy chain constant region and/or a light chain constant region. In some embodiments, the constant region is a human IgG1 constant region, a human IgG2 constant region, a human IgG3 constant region, or a human IgG4 constant region. In some embodiments, the heavy chain constant region can be a modified constant region. Exemplary constant regions substitutions and/or modifications are described herein, and include, but are not limited to, substitutions at one or more of the following positions: 234, 235, 265, and 435 (EU index according to Kabat). In some embodiments, an antibody comprising the CDR and/or variable region sequences of AbC can further comprise an IgG1 heavy chain constant region containing one or more of the following substitutions: L234A, L235A, D265C, and H435A (EU index according to Kabat). In some embodiments, an antibody comprising the CDR and/or variable region sequences of AbC can further comprise a heavy chain constant region set forth in SEQ ID NO:102, SEQ ID NO:103, SEQ ID NO:104, SEQ ID NO:105 or SEQ ID NO:106. In some embodiments the antibody comprises alight chain constant region set forth in SEQ ID NO:101.

In certain embodiments, the anti-CD45 antibody, or antigen binding portion thereof, comprises a heavy chain comprising the amino acid sequence as set forth in SEQ ID NO:99. In certain embodiments, an antibody comprises a modified heavy chain (HC) region comprising an HC domain comprising SEQ ID NO:99, or a variant of SEQ ID NO:99, which variant (i) differs from SEQ ID NO:99 in 1, 2, 3, 4 or 5 amino acids substitutions, additions or deletions; (ii) differs from SEQ ID NO:99 in at most 5, 4, 3, 2, or 1 amino acids substitutions, additions or deletions; (iii) differs from SEQ ID NO:99 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:99, wherein in any of (i)-(iv), an amino acid substitution may be a conservative amino acid substitution or a non-conservative amino acid substitution; and wherein the modified heavy chain region retains the CD45 specificity of the antibody (i.e., specificity similar to AbC), or has an enhanced biological activity relative to that of another AbC heavy chain region.

In certain embodiments, the anti-CD45 antibody, or antigen binding portion thereof, comprises a light chain comprising the amino acid sequence as set forth in SEQ ID NO:100. In certain embodiments, an antibody comprises a modified light chain (LC) region comprising an LC domain comprising SEQ ID NO:100, or a variant of SEQ ID NO:100, which variant (i) differs from SEQ ID NO:100 in 1, 2, 3, 4 or 5 amino acids substitutions, additions or deletions; (ii) differs from SEQ ID NO:100 in at most 5, 4, 3, 2, or 1 amino acids substitutions, additions or deletions; (iii) differs from SEQ ID NO:100 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO:100, wherein in any of (i)-(iv), an amino acid substitution may be a conservative amino acid substitution or a non-conservative amino acid substitution; and wherein the modified light chain region retains the CD45 specificity of the antibody (i.e., specificity similar to AbC), or has an enhanced biological activity relative to that of another AbC light chain region.

In some embodiments, antibodies comprising the CDR regions and/or variable regions of AbC can be incorporated into antibody-drug conjugates, as described herein. In addition, antibodies comprising the CDR regions and/or variable regions of AbC can be used in the methods described herein, e.g., for depletion of CD45+ cells in a subject.

Anti-CD45 antibodies disclosed in US patent application publication no. 2022/0267441, incorporated by reference herein, can also be used in the ADCs described herein.

### Consensus CDRs

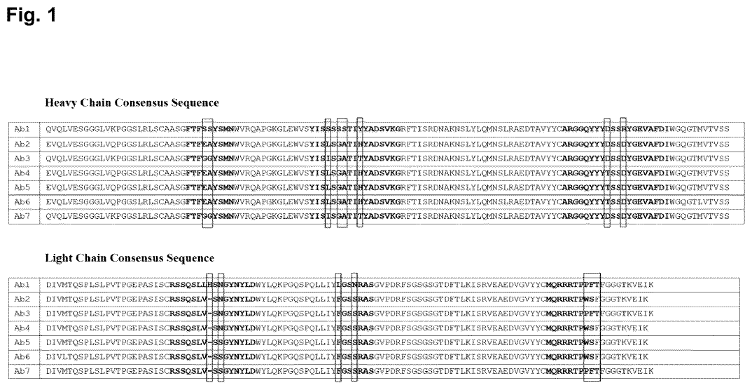

A comparison of the amino acid sequences of the CDRs of Ab1, Ab2, Ab3, Ab4, Ab5, Ab6, and Ab7 is provided as FIG. 1. These antibodies bind to the same epitope on human CD45, and share certain consensus residues in their CDR regions. Consensus heavy chain amino acid CDR sequences are presented in SEQ ID NO:119, SEQ ID NO:120, and SEQ ID NO:121; and consensus light chain amino acid CDR sequences are presented in SEQ ID NO:122, SEQ ID NO:123, and SEQ ID NO:124.

Accordingly, in some embodiments, an isolated anti-CD45 antibody, or antigen-binding portion thereof, comprises a heavy chain variable region comprising a CDR1 domain comprising the amino acid sequence as set forth in SEQ ID NO:119, a CDR2 domain comprising the amino acid sequence as set forth in SEQ ID NO:120, and a CDR3 domain comprising the amino acid sequence as set forth in SEQ ID NO:121; and a light chain variable region comprising a CDR1 domain comprising the amino acid sequence as set forth in SEQ ID NO:122, a CDR2 domain comprising the amino acid sequence as set forth in SEQ ID NO:123; and a CDR3 domain comprising the amino acid sequence as set forth in SEQ ID NO:124. The foregoing antibody can, in some embodiments, further comprise a heavy chain constant region and/or a light chain constant region. For example, in some embodiments, the foregoing antibody can further comprise a heavy chain constant region selected from that set forth in any one of SEQ ID NO:102, SEQ ID NO:103, SEQ ID NO:104, SEQ ID NO:105, or SEQ ID NO:106, and/or a light chain constant region set forth in SEQ ID NO:101.

### Fc-Modified Antibodies

Contemplated herein are antibodies, or antigen-binding fragments thereof, capable of binding CD45 and having Fc modifications that allow Fc silencing, where such antibodies, or antigen-binding fragments thereof, can be used as therapeutic agents alone or as ADCs to deplete cells expressing CD45 in a patient in need thereof. For example, in some embodiments, such antibodies, or antigen-binding fragments thereof, contemplated herein may be used to deplete certain cell types, including HSCs and leukocytes. Thus, in certain embodiments, the antibodies, or antigen-binding fragments thereof, contemplated herein may be used to condition a patient for HSC transplant. In some embodiments, the antibodies, or antigen-binding fragments thereof, contemplated herein may be used to reset the immune system of a patient by, for example, depleting HSCs and leukocytes in the patient and administering an HSC transplant to the patient. In some embodiments, the antibodies, or antigen-binding fragments thereof, contemplated herein may be used to treat a disease associated with CD45 positive cells, including but not limited to cancer and autoimmune disease, by eliminating disease-causing CD45+ cells from the patient.

For example, contemplated herein are antibodies, or antigen-binding fragments thereof, capable of binding an antigen expressed by hematopoietic stem cells, such as CD45, and having Fc modifications that allow Fc silencing, where such antibodies, or antigen-binding fragments thereof, can be used as therapeutic agents alone or as ADCs to (i) treat cancers and autoimmune diseases characterized by CD45+ hematopoietic stem cells; and (ii) promote the engraftment of transplanted hematopoietic stem cells in a patient in need of transplant therapy. These therapeutic activities can be caused, for instance, by the binding of an anti-CD45 antibody, or antigen-binding fragment thereof, that binds to expressed by a hematopoietic cell (e.g., hematopoietic stem cell or mature immune cell (e.g., T cell)), such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein. The Fc-modified antibodies and ADCs contemplated herein not only allow for selective depletion of endogenous hematopoietic stem cells but also have reduced cytotoxic effects on the exogenous hematopoietic stem cell transplant, thereby further promoting engraftment of the hematopoietic stem cell graft.

The antibodies or binding fragments described herein may also include modifications and/or mutations that alter the properties of the antibodies and/or fragments, such as those that increase or decrease half-life, or increase or decrease ADCC. In one embodiment, antibodies comprising one or more radiolabeled amino acids are provided. A radiolabeled antibody may be used for both diagnostic and therapeutic purposes (conjugation to radiolabeled molecules is another possible feature). Non-limiting examples of labels for polypeptides include, but are not limited to 3H, 14C, 15N, 35S, 90Y, 99Tc, and 125I, 131I, and 186Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, 5,102,990 (U.S. Pat. No. RE35,500), U.S. Pat. Nos. 5,648,471 and 5,697,902. For example, a radioisotope may be conjugated by a chloramine T method.

In one embodiment, the anti-CD45 antibody, or binding fragment thereof, comprises a modified Fc region, wherein said modified Fc region comprises at least one amino acid modification relative to a wild-type Fc region, such that said molecule has an altered affinity for or binding to an FcgammaR (FcγR). Certain amino acid positions within the Fc region are known through crystallography studies to make a direct contact with FcγR. Specifically amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). The antibodies described herein may comprise variant Fc regions comprising modification of at least one residue that makes a direct contact with an FcγR based on structural and crystallographic analysis. In one embodiment, the Fc region of the anti-CD45 antibody (or fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” refers to the numbering of the human IgG1 EU antibody. In one embodiment, the Fc region comprises a D265A mutation. In one embodiment, the Fc region comprises a D265C mutation. In some embodiments, the Fc region of the antibody (or fragment thereof) comprises an amino acid substitution at amino acid 234 according to the EU index as in Kabat.

In one embodiment, the Fc region comprises a mutation at an amino acid position of D265, V205, H435, I253, and/or H310. For example, specific mutations at these positions include D265C, V205C, H435A, I253A, and/or H310A (Mutations are according to the EU index as in Kabat).

In one embodiment, the Fc region comprises a L234A mutation. In some embodiments, the Fc region of the anti-CD45 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation. In yet a further embodiment, the Fc region comprises a D265C, L234A, L235A, and H435A mutation. In a further embodiment, the Fc region comprises a D265C and H435A mutation (Mutations are according to the EU index as in Kabat).

In some embodiments, the anti-CD45 antibody herein comprises an Fc region comprising one of the following modifications or combinations of modifications: D265A, D265C, D265C/H435A, D265C/LALA, D265C/LALA/H435A, D265C/N297G, D265C/N297G/H435A, D265C (IgG2*), D265C (IgG2)/H435A, D265C/N297Q/H435A, D265C/N297Q, EPLVLAdelG/H435A, N297A, N297G, or N297Q (EU index according to Kabat).

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4^{th} Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least a 70% decrease, at least a 80% decrease, at least a 90% decrease, at least a 95% decrease, at least a 98% decrease, at least a 99% decrease, or about a 100% decrease in binding to a Fc gamma receptor relative to binding of the identical antibody comprising an unmodified Fc region to the Fc gamma receptor (e.g., as assessed by biolayer interferometry (BLI)).

Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in or for their ability to trigger cytokine release, e.g. by human peripheral blood mononuclear cells.

Thus, in one embodiment, the Fc region comprises a mutation resulting in a decrease in half life (e.g., relative to an antibody having an unmodified Fc region). An antibody having a short half life may be advantageous in certain instances where the antibody is expected to function as a short-lived therapeutic, e.g., the conditioning step described herein where the antibody is administered followed by HSCs. Typically, the antibody would be substantially cleared prior to delivery of the HSCs, which also generally express a target antigen (e.g., CD45) but are not the target of the anti-CD45 antibody unlike the endogenous stem cells. In one embodiment, the Fc regions comprises a mutation at position 435 (EU index according to Kabat). In one embodiment, the mutation is an H435A mutation.

In one embodiment, the anti-CD45 antibody described herein has a half-life (e.g., in humans) equal to or less than 24 hours, equal to or less than 23 hours, equal to or less than 22 hours, equal to or less than 21 hours, equal to or less than 20 hours, equal to or less than 19 hours, equal to or less than 18 hours, equal to or less than 17 hours, equal to or less than 16 hours, equal to or less than 15 hours, equal to or less than 14 hours, equal to or less than 13 hours, equal to or less than 12 hours, or equal to or less than 11 hours.

In one embodiment, the anti-CD45 antibody described herein has a half-life (e.g., in humans) of about 1-2 hours, about 1-3 hours, about 1-5 hours, about 1-10 hours, about 5-10 hours, about 5-15 hours, about 10-15 hours, about 10-20 hours, about 15-20 hours, about 15-25 hours, or about 20-25 hours.

In some aspects, the Fc region comprises two or more mutations that confer reduced half-life and reduce an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD45 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation.

Notably, Fc amino acid positions are in reference to the EU numbering index unless otherwise indicated.

The disclosures of each of the foregoing publications are incorporated herein by reference as they pertain to anti-CD45 antibody. Antibodies and antigen-binding fragments that may be used in conjunction with the compositions and methods described herein include the above-described antibodies and antigen-binding fragments thereof, as well as variants of those non-human antibodies and antigen-binding fragments described above and antibodies or antigen-binding fragments that bind the same epitope as those described above, as assessed, for instance, by way of a competitive antigen binding assay.

Methods of engineering antibodies to include any of the Fc modifications herein are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

In certain embodiments, an anti-CD45 antibody, or binding-fragment thereof, described herein may be conjugated to a label. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, 3-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

The foregoing mutations may be included in the Fc region of an anti-CD45 antibody used in an ADC for the methods disclosed herein.

### Nucleic Acids, Vectors, and Host Cells

Also provided herein are nucleic acid molecules (e.g., DNA or mRNA) that comprise a nucleic acid sequence which encodes an anti-CD45 antibody described herein, or an antigen binding portion thereof.

Accordingly, in some embodiments, provided herein is an isolated nucleic acid molecule that encodes a heavy chain variable region comprising heavy chain CDR1, CDR2, and CDR3 of Ab1, Ab2, Ab3, Ab4, Ab5, Ab6, Ab7, AbA, AbB, or AbC. In other embodiments, provided herein is an isolated nucleic acid molecule that encodes a heavy chain variable region of Ab1, Ab2, Ab3, Ab4, Ab5, Ab6, Ab7, AbA, AbB, or AbC. In other embodiments, provided herein is an isolated nucleic acid molecule that encodes a heavy chain of Ab1, Ab2, Ab3, Ab4, Ab5, Ab6, Ab7, AbA, AbB, or AbC.

In some embodiments, provided herein is an isolated nucleic acid molecule that encodes a light chain variable region comprising light chain CDR1, CDR2, and CDR3 of Ab1, Ab2, Ab3, Ab4, Ab5, Ab6, Ab7, AbA, AbB, or AbC. In other embodiments, provided herein is an isolated nucleic acid molecule that encodes a light chain variable region of Ab1, Ab2, Ab3, Ab4, Ab5, Ab6, Ab7, AbA, AbB, or AbC. In other embodiments, provided herein is an isolated nucleic acid molecule that encodes a light chain of Ab1, Ab2, Ab3, Ab4, Ab5, Ab6, Ab7, AbA, AbB, or AbC.

In some embodiments, provided herein is an isolated nucleic acid molecule that encodes a heavy chain variable region comprising heavy chain CDR1, CDR2, and CDR3, and a light chain variable region comprising light chain CDR1, CDR2, and CDR3 of Ab1, Ab2, Ab3, Ab4, Ab5, Ab6, Ab7, AbA, AbB, or AbC. In other embodiments, provided herein is an isolated nucleic acid molecule that encodes a heavy chain variable region and a light chain variable region of Ab1, Ab2, Ab3, Ab4, Ab5, Ab6, Ab7, AbA, AbB, or AbC. In other embodiments, provided herein is an isolated nucleic acid molecule that encodes a heavy chain and a light chain of Ab1, Ab2, Ab3, Ab4, Ab5, Ab6, Ab7, AbA, AbB, or AbC.

A nucleic acid encoding an antibody heavy chain, or a portion thereof, may be present in the same nucleic acid molecule (e.g., expression vector) as a nucleic acid encoding an antibody light chain, or a portion thereof. Alternatively, the heavy and light chain sequences may be present on separate nucleic acid molecules (e.g., separate expression vectors).

In one embodiment, the disclosure provides an isolated nucleic acid molecule comprising a nucleic acid sequence set forth in any one of SEQ ID NO:125; SEQ ID NO:126; SEQ ID NO:127; SEQ ID NO:128; SEQ ID NO:129; SEQ ID NO:130; SEQ ID NO:131; SEQ ID NO:132; SEQ ID NO:133; SEQ ID NO:134; SEQ ID NO:135; SEQ ID NO:136; SEQ ID NO:137; SEQ ID NO:138; SEQ ID NO:139; SEQ ID NO:140; SEQ ID NO:141; SEQ ID NO:142; SEQ ID NO:143; and/or SEQ ID NO:144; wherein the isolated nucleic acid encodes an anti-CD45 antibody, or a portion thereof.

In another embodiment, the disclosure provides an isolated nucleic acid molecule comprising a nucleic acid sequence set forth in any one of SEQ ID NO:150; SEQ ID NO:151; SEQ ID NO:152; SEQ ID NO:153; SEQ ID NO:154; SEQ ID NO:155; SEQ ID NO:156; SEQ ID NO:157; SEQ ID NO:158; SEQ ID NO:159; SEQ ID NO:160; and/or SEQ ID NO:161; wherein the isolated nucleic acid encodes an anti-CD45 antibody, or a portion thereof.

An expression vector may contain a nucleic acid(s) encoding the heavy chain and/or a light chain of an anti-CD45 antibody. For example, an expression vector may comprise the nucleotide sequence set forth in SEQ ID NO: 162 which encodes an anti-CD45 antibody heavy chain and/or the nucleotide sequence set forth in SEQ ID NO: 163 which encodes an anti-CD45 antibody light chain.

Antibodies may be produced using recombinant methods and compositions, e.g., as described in U.S. Pat. No. 4,816,567. In one embodiment, isolated nucleic acid encoding an anti-CD45 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD45 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-CD45 antibody, or antigen binding fragment thereof, comprises variable regions having an amino acid sequence that is at least 95%, 96%, 97% or 99% identical to the SEQ ID Nos disclosed herein. Alternatively, the anti-CD45 antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

In one embodiment, the anti-CD45 antibody, or antigen binding fragment thereof, comprises a heavy chain variable region and a heavy chain constant region having an amino acid sequence that is disclosed herein. In another embodiment, the anti-CD45 antibody, or antigen binding fragment thereof, comprises a light chain variable region and a light chain constant region having an amino acid sequence that is disclosed herein. In yet another embodiment, the anti-CD45 antibody, or antigen binding fragment thereof, comprises a heavy chain variable region, a light chain variable region, a heavy chain constant region and a light chain constant region having an amino acid sequence that is disclosed herein.

### Methods of Identifying Antibodies

Provided herein are novel anti-CD45 antibodies that may be used, for example, to deplete CD45+ cells in a patient. These antibodies can be useful, e.g., in conditioning methods for stem cell transplantation or the transplantation of genetically-modified cells. In view of the disclosure provided herein, other anti-CD45 antibodies can be identified.

Methods for high throughput screening of antibody, or antibody fragment libraries capable of binding CD45 expressed by hematopoietic stem can be used to identify anti-CD45 antibodies useful for treating cancers, autoimmune diseases, and conditioning a patient (e.g., a human patient) in need of hematopoietic stem cell therapy as described herein. Such methods can be used to identify improved versions of the anti-CD45 antibodies described herein. Such methods include in vitro display techniques known in the art, such as phage display, bacterial display, yeast display, mammalian cell display, ribosome display, mRNA display, and cDNA display, among others.

The use of phage display to isolate antibodies, or antigen-binding fragments, that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). Human anti-CD45 antibodies can also be generated, for example, in the HuMAb-Mouse® or XenoMouse™. These techniques, among others, can be used to identify and improve the affinity of antibodies, antibody or fragments, capable of binding CD45 expressed by hematopoietic stem cells in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies capable of binding an antigen (e.g., CD45) expressed by hematopoietic stem cells. For example, using computational modeling techniques, one of skill in the art can screen libraries of antibodies, or antibody fragments, in silico for molecules capable of binding specific epitopes on an antigen expressed by hematopoietic stem cells (e.g., CD45), such as extracellular epitopes of the antigen.

Additional techniques can be used to identify antibodies, or antibody fragments, capable of binding CD45 expressed by hematopoietic stem cells and that are internalized by the cell, for instance, by receptor-mediated endocytosis. For example, the in vitro display techniques described above can be adapted to screen for antibodies, or antibody fragments, that bind CD45 and that are subsequently internalized. Phage display represents one such technique that can be used in conjunction with this screening paradigm. To identify an anti-CD45 antibody, or antibody fragment, that can be internalized by hematopoietic stem cells, one of skill in the art can use the phage display techniques described in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with CD45 for instance, by first incubating the phage library with blocking agents (such as, for instance, milk protein, bovine serum albumin, and/or IgG so as to remove phage encoding antibodies, or antibody fragments, that exhibit non-specific protein binding and phage that encode antibodies or fragments thereof that bind Fc domains, and then incubating the phage library with a population of cells, e.g., hematopoietic stem cells, which express CD45. The phage library can be incubated with the hematopoietic stem cells for a time sufficient to allow anti-CD45 antibodies, or antibody fragments, to bind the cognate cell-surface antigen and to subsequently be internalized by the hematopoietic stem cells (e.g., from 30 minutes to 6 hours at 4° C., such as 1 hour at 4° C.). Phage containing antibodies, or antibody fragments, that do not exhibit sufficient affinity for the CD45 so as to permit binding to, and internalization by, hematopoietic stem cells can subsequently be removed by washing the cells, for instance, with cold (4° C.) 0.1 M glycine buffer at pH 2.8. Phage bound to antibodies, or antibody fragments, that have been internalized by the hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or antibody fragments, inserted within the phage genome. The encoded antibodies, or antibody fragments, can subsequently be prepared de novo by chemical synthesis (for instance, of antibody fragments, such as scFv fragments) or by recombinant expression (for instance, of full-length antibodies).

The internalizing capacity of the prepared antibodies, or antibody fragments, can be assessed, for instance, using radionuclide internalization assays known in the art. For example, anti-CD45 antibodies, or antibody fragments, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or antibody fragments, using beads, such as polystyrene beads, containing electrophilic halogen reagents (e.g., Iodination Beads, Thermo Fisher Scientific, Inc., Cambridge, MA). Radiolabeled antibodies, fragments thereof, or ADCs, can be incubated with hematopoietic stem cells for a time sufficient to permit internalization (e.g., from 30 minutes to 6 hours at 4° C., such as 1 hour at 4° C.). The cells can then be washed to remove non-internalized antibodies or fragments thereof, (e.g., using cold (4° C.) 0.1 M glycine buffer at pH 2.8). Internalized antibodies, or antibody fragments, can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs.

Antibodies may be produced using recombinant methods and compositions, e.g., as described in U.S. Pat. No. 4,816,567. In one embodiment, isolated nucleic acid encoding an anti-CD45 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD45 antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

### Cytotoxins and Linkers

Anti-CD45 antibodies, or antigen-binding fragments thereof, described herein can be conjugated (linked) to a cytotoxin via a linker. In some embodiments, the cytotoxic molecule is conjugated to a cell internalizing antibody, or antigen-binding fragment thereof as disclosed herein such that following the cellular uptake of the antibody, or fragment thereof, the cytotoxin may access its intracellular target and mediate hematopoietic cell death. In certain embodiments, an anti-CD45 scFv comprising VH and VL variable regions described herein (or variable regions comprising light chain and heavy chain CDR sets described herein) are conjugated to a toxin to form an scFv toxin.

### Cytotoxins

Various cytotoxins can be conjugated to an anti-CD45 antibody via a linker for use in the therapies described herein. In particular, the anti-CD45 ADCs include an anti-CD45 antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

ADCs of the present disclosure therefore may be of the general formula

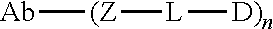

wherein an anti-CD45 antibody or antigen-binding fragment thereof (Ab) is conjugated (covalently linked) to linker (L), through a chemical moiety (Z), to a cytotoxic moiety (“drug,” D, or “Cy”), such as an indolinobenzodiazepine pseudo dimer.

Accordingly, the anti-CD45 antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 1. In some embodiments, n is about 2.

In one embodiment, an IGN pseudo dimer described herein (e.g., DGN549) is conjugated to an anti-CD45 antibody such that the ratio of toxin:antibody is about 2. In certain embodiments, the average number of toxins attached to the anti-CD45 antibody is 1.8 to 2.0. In certain embodiments, the average number of toxins attached to the anti-CD45 antibody is 1.7 to 2.1. In certain embodiments, the average number of toxins attached to the anti-CD45 antibody is 1.6 to 2.2. In one embodiment, an IgG1 anti-CD45 antibody is conjugated to two IGN pseudo dimers, wherein the anti-CD45 antibody comprises a heavy chain variable region comprising a CDR1 domain comprising the amino acid sequence as set forth in SEQ ID NO:42, a CDR2 domain comprising the amino acid sequence as set forth in SEQ ID NO:43, and a CDR3 domain comprising the amino acid sequence as set forth in SEQ ID NO:44; and comprises a light chain variable region comprising a CDR1 domain comprising the amino acid sequence as set forth in SEQ ID NO:46, a CDR2 domain comprising the amino acid sequence as set forth in SEQ ID NO:47; and a CDR3 domain comprising the amino acid sequence as set forth in SEQ ID NO:48. In a preferred embodiment, the toxin is conjugated to an anti-CD45 antibody via a cysteine residue in the Fc region of the antibody and via a linker, such as a protease-cleavable linker.

In a preferred embodiment, the anti-CD45 antibody of the ADC is Ab5, or an antibody having CDR and/or variable region sequences of Ab5.

The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-CD45 ADCs, they may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a DNA alkylating agent. In some embodiments, the DNA alkylating agent is an indolinobenzodiazepine, an indolinobenzodiazepine dimer, or a variant thereof, or another cytotoxic compound described herein or known in the art. In certain embodiments, the IGN is DGN549.

Additional details regarding cytotoxins that can be used in the anti-CD45 ADCs useful in the compositions and methods of the present disclosure are described below.

### Benzodiazepines

In some embodiments, the anti-CD45 antibodies, or antigen-binding fragments thereof, described herein can be conjugated to a cytotoxin that comprises a benzodiazepine moiety, such as an IGN, as described herein.

### Indolinobenzodiazepines (IGNs)

In a preferred embodiment, an antibody, or antigen-binding fragment thereof, that binds to CD45 as described herein can be conjugated to a cytotoxin that is an indolinobenzodiazepine (“IGN”) or a cytotoxin that comprises an IGN. In some embodiments, the IGN cytotoxin is an indolinobenzodiazepine dimer or an indolinobenzodiazepine pseudo dimer. In a preferred embodiment, the indolinobenzodiazepine is an indolinobenzodiazepine pseudo dimer.

Indolinobenzodiazepine dimers represent a relatively new chemical class of cytotoxins with high in vitro potency (low pM range IC₅₀ values) towards cancer cells. Similar to the PBD dimer SJG-136, IGN dimers bind to the minor groove of DNA, and covalently bind to guanine residues via the two imine functionalities in the dimer, resulting in crosslinking of the DNA. An IGN dimer (IGN 6; replacing the methylene groups of the PBD moiety with phenyl rings) demonstrated ˜10-fold higher potency in vitro as compared to SJG-136, possibly due to faster rate of adduct formation with DNA IGN (see, e.g., Miller et al., “A New Class of Antibody-Drug Conjugates with Potent DNA Alkylating Activity” Mol. Cancer Ther. 2016, 15(8), 1870-1878). In contrast, IGN pseudo dimers comprise a single reactive indolinobenzodiazepine imine; the second indolinobenzodiazepine in the dimeric cytotoxin is present in reduced (amine) form. Accordingly, IGN pseudo dimers alkylate DNA through the single imine moiety present in the dimer, and do not crosslink DNA.

In some embodiments, the cytotoxin is an IGN pseudo dimer having a structure of formula:

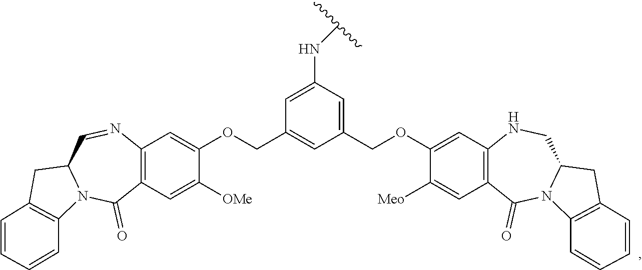

wherein the wavy line indicates the attachment point of the linker. The foregoing structure is also referenced as DGN549.

In some embodiments, the cytotoxin is a sulfonated IGN pseudo dimer having a structure of formula:

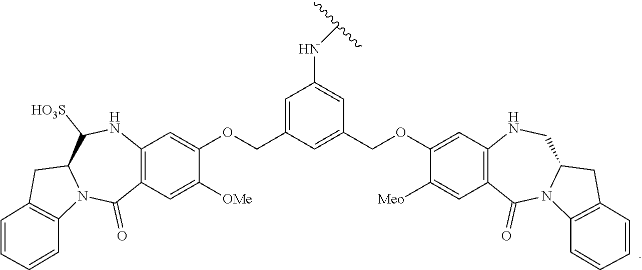

In some embodiments, the cytotoxin-linker conjugate, prior to conjugation to the antibody and including the reactive substituent Z′, taken together as Cy-L-Z′, has the structure:

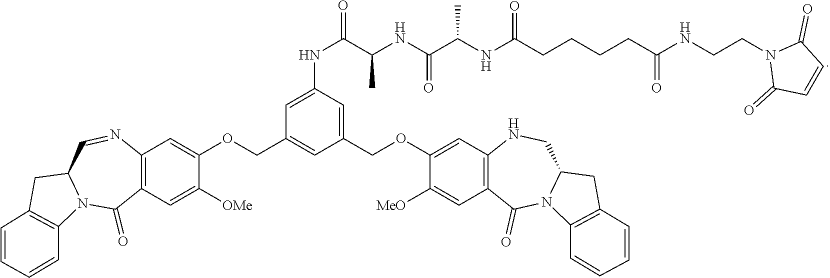 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

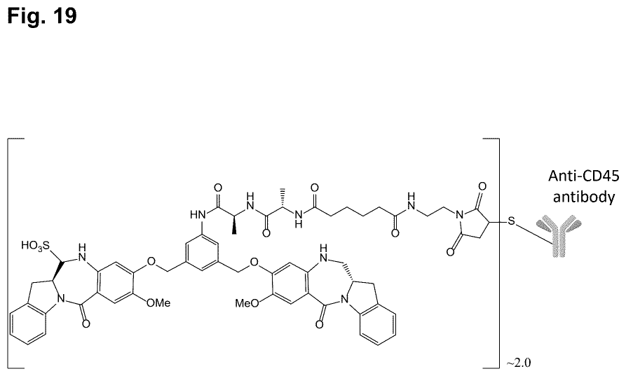

The foregoing cytotoxin-linker conjugate is referred to herein as DGN549-C (also referred to herein as DGN549) and is described in FIG. 19 (noting that the DAR of 2 in FIG. 19 is exemplary). DGN549 is present in the ADC IMGN632, which is disclosed in, for example, International Patent Application Publication No. WO2017004026, which is incorporated by reference herein. Conjugation of DGN549-C to an antibody is described in Bai et al. (2020) Bioconjugate Chem 31: 93-103.

In one aspect, an anti-CD45-IGN ADC disclosed herein comprises a sulfonated DGN549, wherein the cytotoxin is reversibly sulfonated at the imine moiety. Sulfonation of DGN549 can be achieved by exposure to high concentrations of bisulfite salt. This reversible sulfonation, which is maintained in the presence of excess bisulfite salts, enhances the water solubility of an IGN molecule (Miller et al. (2016) Mo Cancer Ther 15(8): 1870-1878). The sulfonation of the imine moiety of DGN549 is reversible in the absence of excess concentrations of the bisulfite salts. Miller et al. (2016) Mo Cancer Ther 15(8): 1870-1878 reported that a sulfonated IGN spontaneously reversed to the free imine ex vivo in human plasma.

In some embodiments, the cytotoxin-linker conjugate, prior to conjugation to the antibody and including the reactive substituent Z′, taken together as Cy-L-Z′, has the structure:

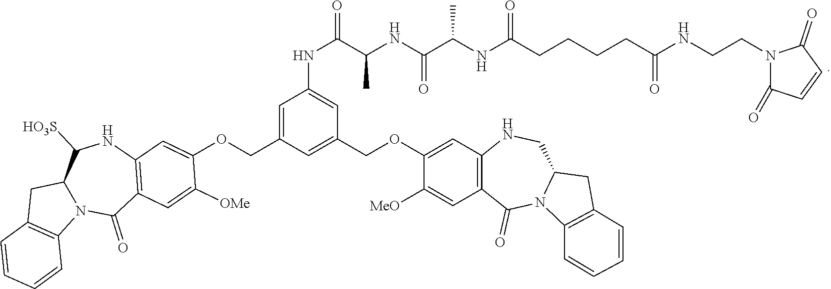

In a preferred embodiment, an anti-CD45 ADC used in the methods disclosed herein has one of the following formulas:

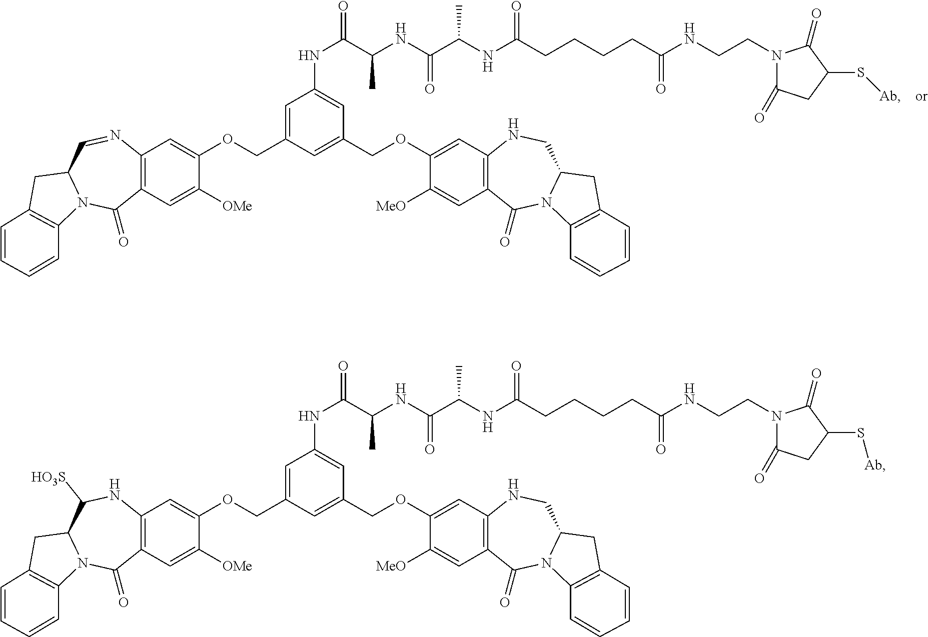

In some embodiments, the cytotoxin is an indolinobenzodiazepine pseudo dimer having a structure of formula:

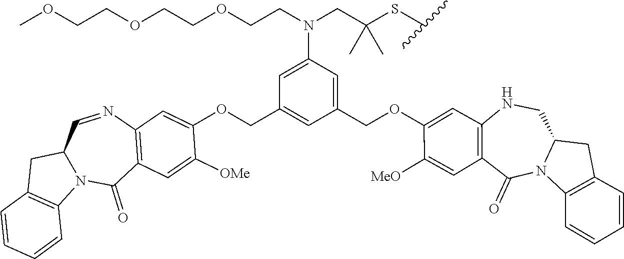 [SMILES: *SC(C)(C)CN(CCOCCOCCOC)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker. This IGN pseudo dimer cytotoxin is referred to herein as DGN462, disclosed in, for example, U.S. Patent Application Publication No. 20170080102, which is incorporated by reference herein.

In some embodiments, the cytotoxin-linker conjugate, prior to conjugation to the antibody and including the chemical moiety Z, taken together as Cy-L-Z, has the structure:

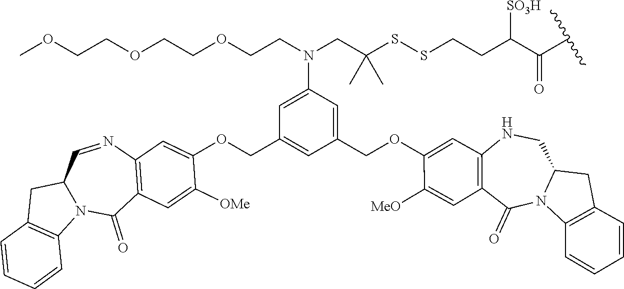 [SMILES: *C(=O)C(CCSSC(C)(C)CN(CCOCCOCCOC)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1)S(=O)(=O)O]

wherein the wavy line indicates the point of attachment to the antibody (e.g., an anti-CD45 antibody or fragment thereof). This cytotoxin-linker conjugate is present in the ADC IMGN779, disclosed in, for example, U.S. Patent Application Publication No. 20170080102, previously incorporated by reference herein.

### Linkers

The term “Linker” as used herein means a divalent chemical moiety comprising a covalent bond or a chain of atoms that covalently attaches an anti-CD45 antibody to a cytotoxin to form an anti-CD45 antibody drug conjugate (ADC), each as described herein. Suitable linkers have two reactive termini, one for conjugation to an antibody and the other for conjugation to a cytotoxin. The antibody conjugation reactive terminus of the linker (reactive moiety, Z′) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as reactive moiety Z′, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

A variety of linkers can be used to conjugate the antibodies, or antibody fragments, described to a cytotoxic molecule. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, or antibody fragments, described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ— units, wherein p is an integer from 1-6, independently selected for each occasion.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

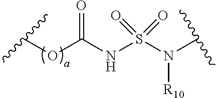

wherein a is 0 or 1; and
R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like.

In some embodiments, the linker L comprises the moiety *-L₁L₂-**, wherein:
L₁ is absent or is —(CH₂)ₘNR¹³C(═O)—, —(CH₂)ₘNR¹³—, —(CH₂)ₘX₃(CH₂)ₘ—,

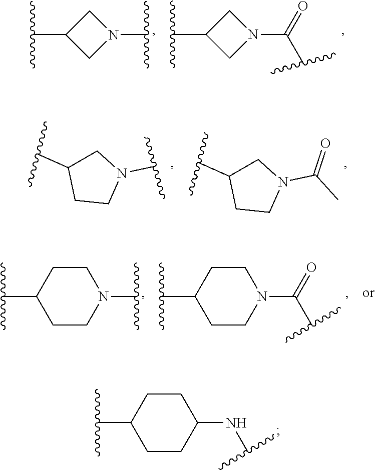

L₂ is absent or is —(CH₂)ₘ—, —NR¹³(CH₂)ₘ—, —(CH₂)ₘNR¹³C(═O)(CH₂)ₘ—, —X₄, —(CH₂)ₘNR¹³C(═O)X₄, —(CH₂)ₘNR¹³C(═O)—, —((CH₂)ₘO)ₙ(CH₂)ₘ—, —((CH₂)ₘO)ₙ(CH₂)ₘX₃(CH₂)ₘ—, NR¹³((CH₂)ₘO)ₙX₃(CH₂)ₘ—, —NR¹³((CH₂)ₘO)ₙ(CH₂)ₘX₃(CH₂)ₘ—, —X₁X₂C(═O)(CH₂)ₘ—, —(CH₂)ₘ(O(CH₂)ₘ)ₙ—, —(CH₂)ₘNR¹³(CH₂)ₘ—, —(CH₂)ₘNR¹³C(═O)(CH₂)ₘX₃(CH₂)ₘ—, —(CH₂)ₘC(═O)NR¹³(CH₂)ₘNR¹³C(═O)(CH₂)ₘ—, —(CH₂)ₘC(═O)—, —(CH₂)ₘNR¹³(CH₂)ₘC(═O)X₂X₁C(═O)—, —(CH₂)ₘX₃(CH₂)ₘC(═O)X₂X₁C(═O)—, —(CH₂)ₘC(═O)NR¹³(CH₂)ₘ—, —(CH₂)ₘC(═O)NR¹³(CH₂)ₘX₃(CH₂)ₘ—, —(CH₂)ₘX₃(CH₂)ₘNR¹³C(═O)(CH₂)ₘ—, —(CH₂)ₘX₃(CH₂)ₘC(═O)NR¹³(CH₂)ₘ—, —(CH₂)ₘO)ₙ(CH₂)ₘNR¹³C(═O)(CH₂)ₘ—, —(CH₂)ₘC(═O)NR¹³(CH₂)ₘ(O(CH₂)ₘ)ₙ—, —(CH₂)ₘ(O(CH₂)ₘ)ₙC(═O)—, —(CH₂)ₘNR¹³(CH₂)ₘC(═O)—, —(CH₂)ₘC(═O)NR¹³(CH₂)ₘNR¹³C(═O)—, —(CH₂)ₘ(O(CH₂)ₘ)ₙX₃(CH₂)ₘ—, —(CH₂)ₘX₃((CH₂)ₘO)ₙ(CH₂)ₘ—, —(CH₂)ₘX₃(CH₂)ₘC(═O)—, —(CH₂)ₘC(═O)NR¹³(CH₂)ₘO)ₙ(CH₂)ₘX₃(CH₂)ₘ—, —(CH₂)ₘX₃(CH₂)ₘ(O(CH₂)ₘ)ₙNR¹³C(═O)(CH₂)ₘ—, —(CH₂)ₘX₃(CH₂)ₘ(O(CH₂)ₘ)ₙC(═O)—, —(CH₂)ₘX₃(CH₂)ₘ(O(CH₂)ₘ)ₙ—, —(CH₂)ₘC(═O)NR¹³(CH₂)ₘC(═O)—, —(CH₂)ₘC(═O)NR¹³(CH₂)ₘ(O(CH₂)ₘ)ₙC(═O)—, —((CH₂)ₘO)ₙ(CH₂)ₘNR¹³C(═O)(CH₂)ₘ—, —(CH₂)ₘC(═O)NR¹³(CH₂)ₘC(═O)NR¹³(CH₂)ₘ—, —(CH₂)ₘNR¹³C(═O)(CH₂)ₘNR¹³C(═O)(CH₂) —(CH₂)ₘX₃(CH₂)ₘC(═O)NR¹³—, —(CH₂)ₘC(═O)NR¹³—, —(CH₂)ₘX₃—, —C(R¹³)₂(CH₂)ₘ—, —(CH₂)ₘC(R¹³)₂NR¹³—, —(CH₂)ₘC(═O)NR¹³(CH₂)ₘNR¹³—, —(CH₂)ₘC(═O)NR¹³(CH₂)ₘNR¹³C(═O)NR¹³—, —(CH₂)ₘC(═O)X₂X₁C(═O)—, —C(R¹³)₂(CH₂)ₘNR¹³C(═O)(CH₂)ₘ—, —(CH₂)ₘC(═O)NR¹³(CH₂)ₘC(R¹³)₂NR¹³—, —C(R¹³)₂(CH₂)ₘX₃(CH₂)ₘ—, —(CH₂)ₘX₃(CH₂)ₘC(R¹³)₂NR¹³—, —C(R¹³)₂(CH₂)ₘOC(═O)NR¹³(CH₂)ₘ—, —(CH₂)ₘNR¹³C(═O)O(CH₂)ₘC(R¹³)₂NR¹³—, —(CH₂)ₘX₃(CH₂)ₘNR¹³—, —(CH₂)ₘX₃(CH₂)ₘ(O(CH₂)ₘ)ₙNR¹³—, —(CH₂)ₘNR¹³—, —(CH₂)ₘC(═O)NR¹³(CH₂)ₘ(O(CH₂)ₘ)ₙNR¹³—, —(CH₂)ₘ(O(CH₂)ₘ)ₙNR¹³—, —(CH₂CH₂O)ₙ(CH₂)ₘ—, —(CH₂)ₘ(OCH₂CH₂)ₙ; —(CH₂)ₘO(CH₂)ₘ—, —(CH₂)ₘS(═O)₂—, —(CH₂)ₘC(═O)NR¹³(CH₂)ₘS(═O)₂—, —(CH₂)ₘX₃(CH₂)ₘS(═O)₂—, —(CH₂)ₘX₂X₁C(═O)—, —(CH₂)ₘ(O(CH₂)ₘ)ₙC(═O)X₂X₁C(═O)—, —(CH₂)ₘ(O(CH₂)ₘ)ₙX₂X₁C(═O)—, —(CH₂)ₘX₃(CH₂)ₘX₂X₁C(═O)—, —(CH₂)ₘX₃(CH₂)ₘ(O(CH₂)ₘ)ₙX₂X₁C(═O)—, —(CH₂)ₘX₃(CH₂)ₘC(═O)NR¹³(CH₂)ₘNR¹³C(═O)—, —(CH₂)ₘX₃(CH₂)ₘC(═O)NR¹³(CH₂)ₘC(═O)—, —(CH₂)ₘX₃(CH₂)ₘC(═O)NR¹³(CH₂)ₘ(O(CH₂)ₘ)ₙC(═O)—, —(CH₂)ₘC(═O)X₂X₁C(═O)NR¹³(CH₂)ₘ—, —(CH₂)ₘX₃(O(CH₂)ₘ)ₙC(═O)—, —(CH₂)ₘNR¹³C(═O)((CH₂)ₘO)ₙ(CH₂)ₘ—, —(CH₂)ₘ(O(CH₂)ₘ)ₙC(═O)NR¹³(CH₂)ₘ—, —(CH₂)ₘNR¹³C(═O)NR¹³(CH₂)ₘ— or —(CH₂)ₘX₃(CH₂)ₘNR¹³C(═O)—;
wherein
X₁ is

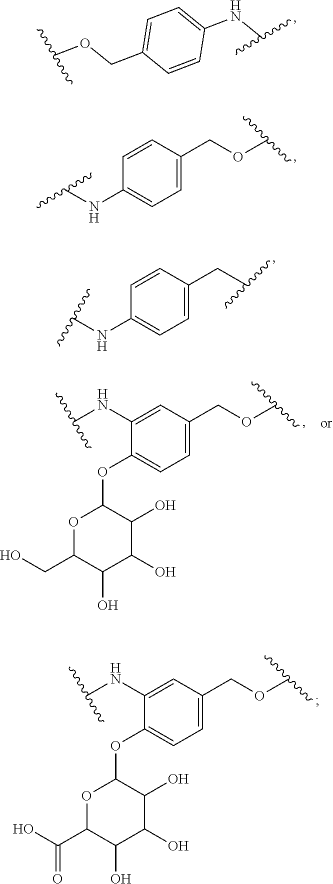 [SMILES: *Nc1ccc(CO*)cc1 ; *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

X₂ is

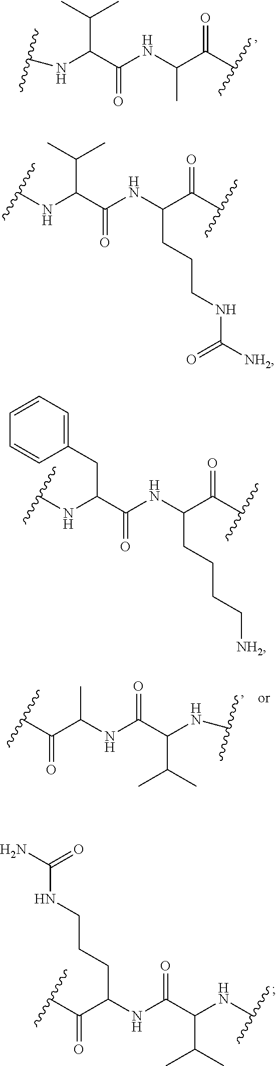

X₃ is

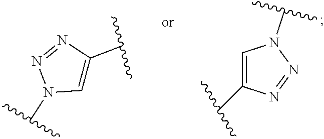 [SMILES: *c1cn(*)nn1 ; *c1cn(*)nn1]

and
X₄ is

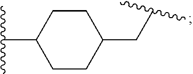

wherein
R¹³ is independently selected for each occasion from H and C₁-C₆ alkyl;
m is independently selected for each occasion from 1, 2, 3, 4, 5, 6, 7, 8, 9 and 10;
n is independently selected for each occasion from 1, 2, 3, 4, 5, 6, 7, 8, 9, 10, 11, 12, 13 and 14; and
wherein the single asterisk (*) indicates the attachment point to the cytotoxin (e.g., an amatoxin), and the double asterisk (**) indicates the attachment point to the reactive substituent Z′ or chemical moiety Z, with the proviso that L₁ and L₂ are not both absent.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker includes —((CH₂)ₙ where n is 6. In some embodiments, L-Z is

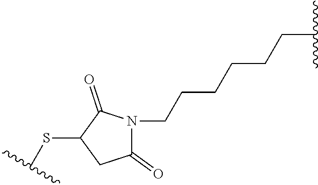

where S is a sulfur atom which represents the reactive substituent present within an antibody, or antigen-binding fragment thereof,

In some embodiments, the linker comprises a —(C═O)(CH₂)ₚ— unit, wherein p is an integer from 1-6.

In one specific embodiment, the linker comprises the structure

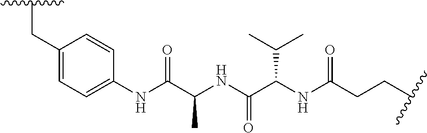 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z′. In another specific embodiment, the linker comprises the structure

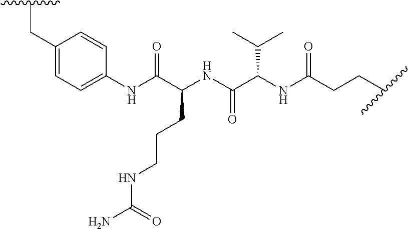 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z′. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein.

In certain embodiments, the linker of the ADC is maleimidocaproyl-Val-Ala-para-aminobenzyl (mc-Val-Ala-PAB).

In certain embodiments, the linker of the ADC is maleimidocaproyl-Val-Cit-para-aminobenzyl (mc-vc-PAB).

In some embodiments, the linker comprises

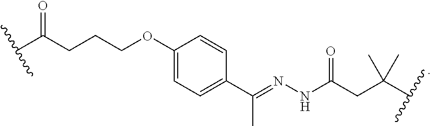 [SMILES: *C(=O)CCCOc1ccc(/C(C)=N/NC(=O)CC(*)(C)C)cc1]

In some embodiments, the linker comprises MCC (4-[N-maleimidomethyl]cyclohexane-1-carboxylate).

In some embodiments, the linker comprises a ((CH₂)ₘO)ₙ(CH₂)ₘ group where n and m are each independently selected from 1, 2, 3, 4, 5, 6, 7, 8, 9, and 10; and a heteroaryl group, wherein the heteroaryl group is a triazole. In some embodiments, the ((CH₂)ₘO)ₙ(CH₂)ₘ— group and triazole together comprise

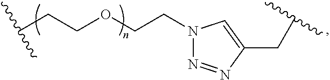

where n is from 1 to 10, and the wavy lines indicate attachment points to additional linker components, the chemical moiety Z, or the amatoxin. Other linkers that may be used in the methods and compositions described herein are described in US 2019/0144504, which is incorporated by reference herein.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

In certain embodiments, an intermediate, which is the precursor of the linker, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

Linkers useful in for conjugation to the antibodies or antigen-binding fragments as described herein include, without limitation, linkers containing chemical moieties Z formed by coupling reactions as depicted in Table 1, below. Curved lines designate points of attachment to the antibody or antigen-binding fragment, and the cytotoxic molecule, respectively.

TABLE 1
Exemplary chemical moieties Z formed by coupling reactions in the formation of
antibody-drug conjugates

| Exemplary | |
|---|---|
| Coupling Reactions | Chemical Moiety Z Formed by Coupling Reactions |
| [3 + 2] Cycloaddition | 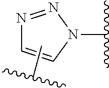 |
| [3 + 2] Cycloaddition | 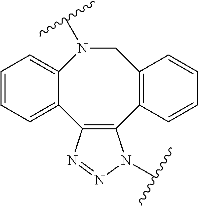 [SMILES: *N1Cc2ccccc2-c2c(nnn2*)-c2ccccc21] |
| [3 + 2] Cycloaddition, Esterification | 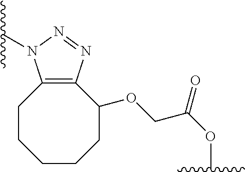 [SMILES: *OC(=O)COC1CCCCCc2c1nnn2*] |
| [3 + 2] Cycloaddition, Esterification | 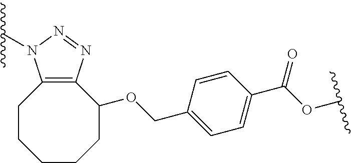 [SMILES: *OC(=O)c1ccc(COC2CCCCCc3c2nnn3*)cc1] |
| [3 + 2] Cycloaddition, Esterification | 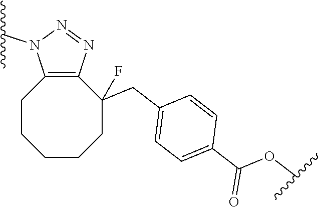 [SMILES: *OC(=O)c1ccc(CC2(F)CCCCCc3c2nnn3*)cc1] |
| [3 + 2] Cycloaddition, Esterification | 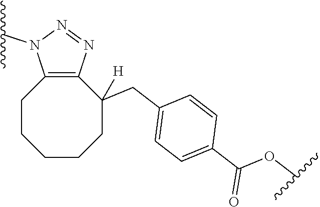 [SMILES: *OC(=O)c1ccc(CC2CCCCCc3c2nnn3*)cc1] |
| [3 + 2] Cycloaddition, Esterification | 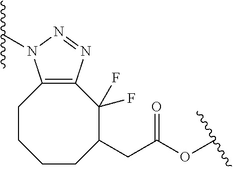 [SMILES: *OC(=O)CC1CCCCc2c(nnn2*)C1(F)F] |
| [3 + 2] Cycloaddition, Esterification | 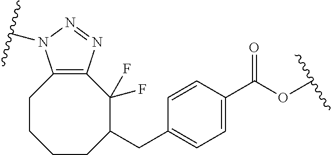 [SMILES: *OC(=O)c1ccc(CC2CCCCc3c(nnn3*)C2(F)F)cc1] |
| [3 + 2] Cycloaddition, Esterification | 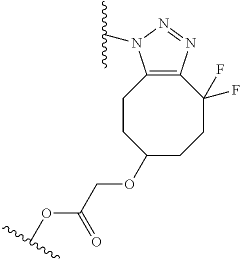 [SMILES: *OC(=O)COC1CCc2c(nnn2*)C(F)(F)CC1] |
| [3 + 2] Cycloaddition, Esterification | 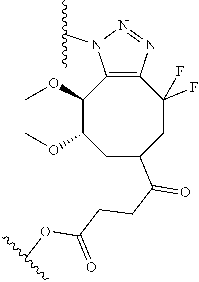 [SMILES: *OC(=O)CCC(=O)C1C[C@H](OC)[C@@H](OC)c2c(nnn2*)C(F)(F)C1] |
| [3 + 2] Cycloaddition, Esterification | 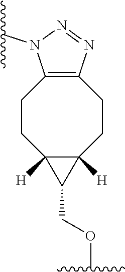 [SMILES: *OC[C@@H]1[C@@H]2CCc3c(nnn3*)CC[C@@H]21] |
| [3 + 2] Cycloaddition, Esterification | 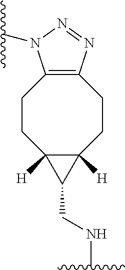 [SMILES: *NC[C@@H]1[C@@H]2CCc3c(nnn3*)CC[C@@H]21] |
| [3 + 2] Cycloaddition, Esterification | 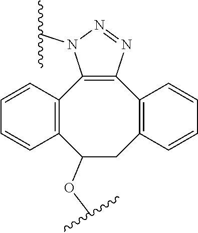 [SMILES: *OC1Cc2ccccc2-c2nnn(*)c2-c2ccccc21] |
| [3 + 2] Cycloaddition, Etherification | 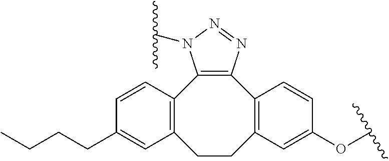 [SMILES: *Oc1ccc2c(c1)CCc1cc(CCCC)ccc1-c1c-2nnn1*] |
| [3 + 2] Cycloaddition | 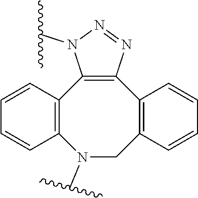 [SMILES: *N1Cc2ccccc2-c2nnn(*)c2-c2ccccc21] |
| Michael addition | 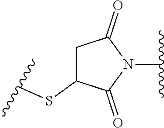 [SMILES: *SC1CC(=O)N(*)C1=O] |
| Michael addition | 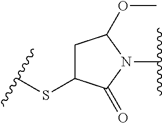 [SMILES: *SC1CC(OC)N(*)C1=O] |
| Imine condensation, Amidation | 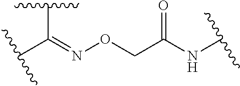 [SMILES: *NC(=O)CON=C(*)*] |
| Imine condensation | 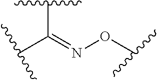 [SMILES: *ON=C(*)*] |
| Disulfide formation | 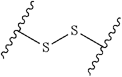 [SMILES: *SS*] |
| Thiol alkylation | 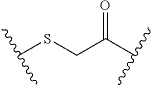 [SMILES: *SCC(*)=O] |
| Condensation, Michael addition | 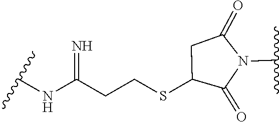 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive moiety Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

In some embodiments, Z′ is —NR¹³C(═O)CH═CH₂, —N₃, —SH, —S(═O)₂(CH═CH₂), —(CH₂)₂S(═O)₂(CH═CH₂), —NR¹³S(═O)₂(CH═CH₂), —NR¹³C(═O)CH₂R¹⁴, —NR¹³C(═O)CH₂Br, —NR¹³C(═O)CH₂I, —NHC(═O)CH₂Br, —NHC(═O)CH₂I, —ONH₂, —C(O)NHNH₂, —CO₂H, —NH₂, —NH(C═O), —NC(═S),

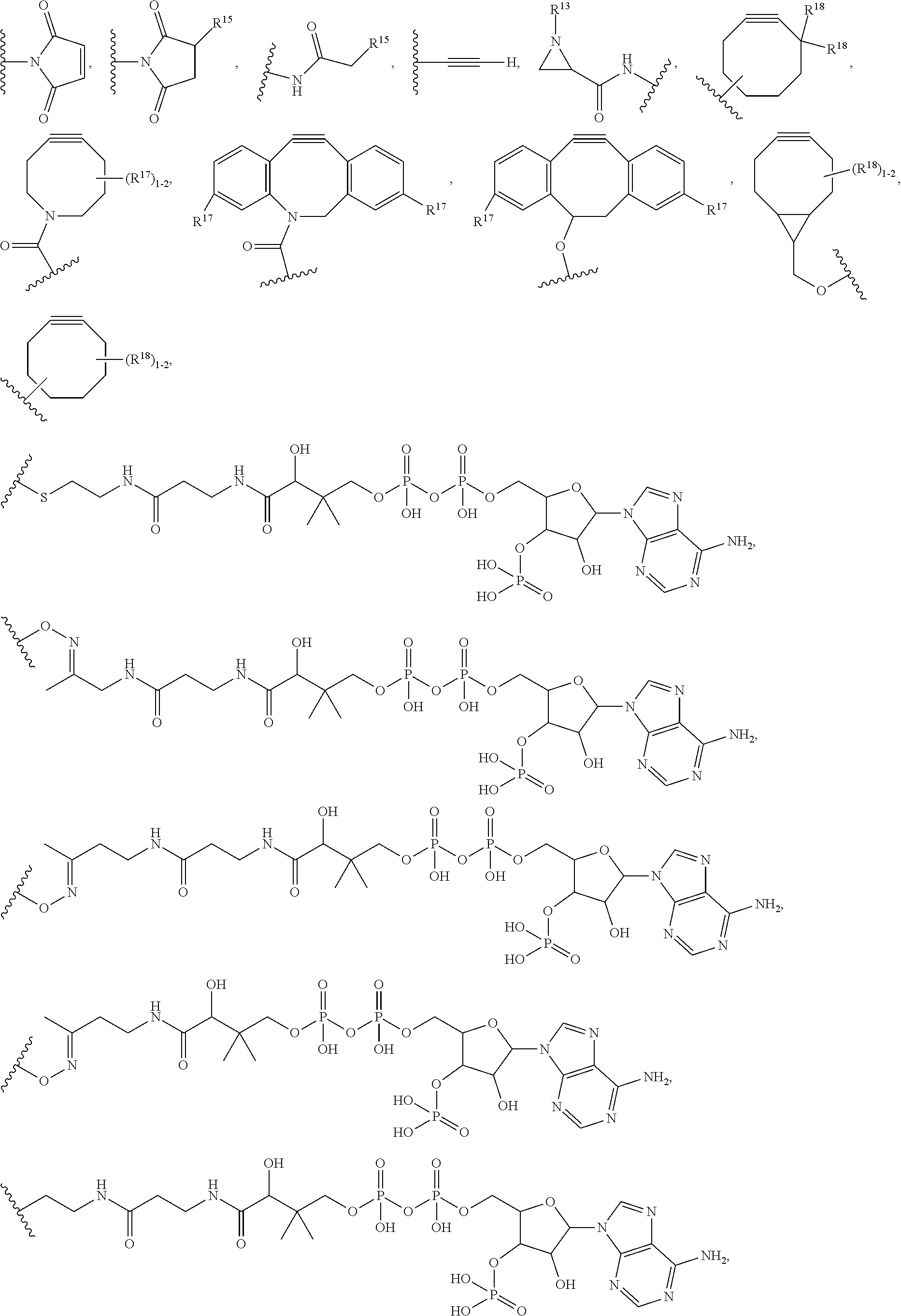

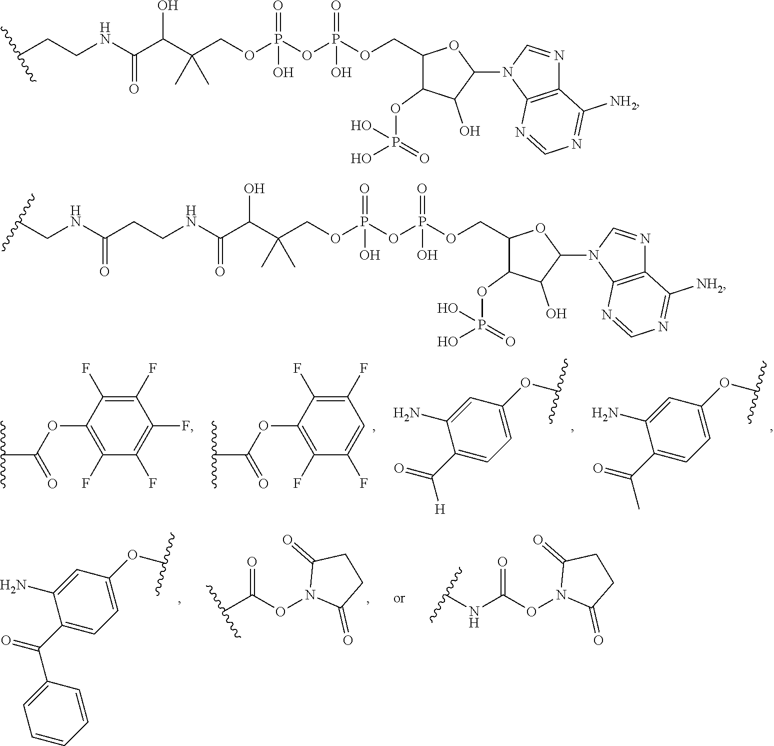

wherein
R¹³ is independently selected for each occasion from H and C₁-C₆ alkyl;
R¹⁴ is —S(CH₂)ₙCHR¹⁵NHC(═O)R¹³;
R¹⁵ is R¹³ or —C(═O)OR¹³;
R¹⁶ is independently selected for each occasion from H, C₁-C₆ alkyl, F, Cl, and —OH;
R¹⁷ is independently selected for each occasion from H, C₁-C₆ alkyl, F, Cl, —NH₂, —OCH₃, —OCH₂CH₃, —N(CH₃)₂, —ON, —NO₂ and —OH; and
R¹⁸ is independently selected for each occasion from H, C₁-C₆ alkyl, F, benzyloxy substituted with —C(═O)OH, benzyl substituted with —C(═O)OH, C₁-C₄ alkoxy substituted with —C(═O)OH, and C₁-C₄ alkyl substituted with —C(═O)OH.

Examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substituents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, and aldehyde, among others.

For instance, linkers suitable for the synthesis of ADCs include, without limitation, reactive substituents Z′ such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (het-ero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis(sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

In some embodiments, the chemical moiety Z is selected from Table 1. In some embodiments, the chemical moiety Z is

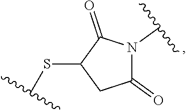

where S is a sulfur atom which represents the reactive substituent present within an antibody, or antigen-binding fragment thereof, that binds CD45 (e.g., from the —SH group of a cysteine residue).

In some embodiments, the linker-reactive substituent group structure L-Z′, prior to conjugation with the antibody or antigen binding fragment thereof, is:

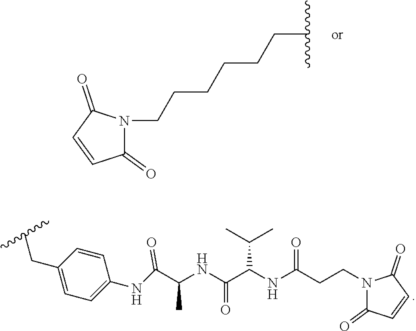

The foregoing linker moieties, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

### Preparation of Antibody-Drug Conjugates

In the ADCs as disclosed herein, an anti-CD45 antibody, or antigen binding fragment thereof, is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z′, followed by reaction with a reactive substituent of an antibody or antigen binding fragment thereof as described herein above. Additional methods for preparing ADC are described herein.

In another aspect, the anti-CD45 antibody, or antigen binding fragment thereof, has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-CD45 antibody, or antigen binding fragment thereof, can have one or more carbohydrate groups that can be chemically modified to have one or more sulfhydryl groups. The ADC is then formed by conjugation through the sulfhydryl group's sulfur atom as described herein above.

In yet another aspect, the anti-CD45 antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

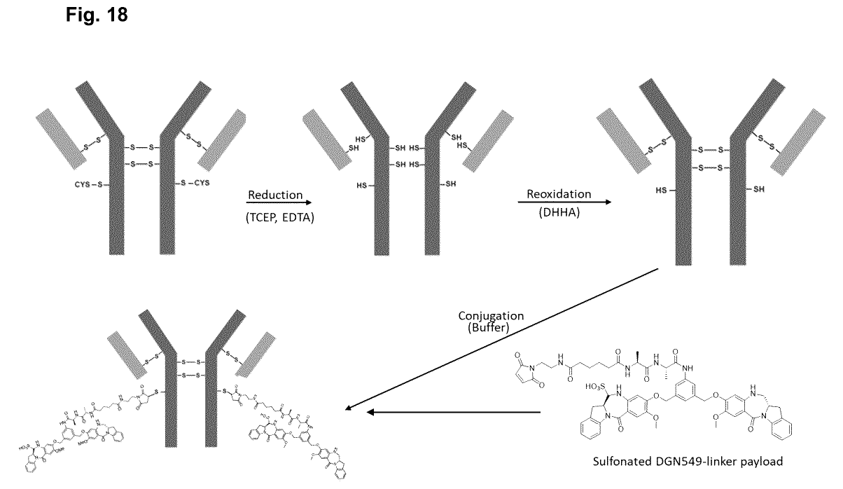

In one embodiment, an ADC of the disclosure comprising an anti-CD45 antibody and a DGN549 cytotoxin is produced through the conjugation of an anti-CD45 antibody to a DGN549 linker-payload via a succinimide linkage. In this embodiment, conjugation is performed via reaction of a maleimide at the linker terminus with an engineered cysteine residue (or pair of engineered cysteine residues) in the antibody. A schematic illustration according to a non-limiting embodiment is provided in FIG. 18. With reference to FIG. 18, the method generally comprises reduction of the anti-CD45 antibody with e.g., tris(2-carboxyethyl)phosphine (TCEP) followed by selective reoxidation of the interchain cysteines to re-form the native IgG1 disulfide bonds using, e.g., dehydroascorbic acid (DHAA). The engineered cysteines (at amino acid position 265 of the Fc region (EU numbering per Kabat) remain reduced and are then conjugated with the DGN549-linker payload conjugate (DGN549-C, which is DGN549 with a protease cleavable linker). In some embodiments, and as illustrated in FIG. 18, in order to enhance solubility of the cytotoxin linker conjugate, and ADC, the DGN549 cytotoxin is reversibly sulfonated by exposure to excess sodium bisulfite and is maintained in the sulfonated state by including excess bisulfite in a buffered conjugation medium. This general methodology is disclosed in, for example, U.S. Pat. No. 10,287,256 to Hilderbrand et al., and is incorporated by reference herein with respect to preparation of ADC's. The formula of sulfonated DGN549 anti-CD45 ADC is described in FIG. 19.

### Therapeutic Uses

CD45 is an important cell surface molecule broadly expressed throughout the hematopoietic and immune systems. Anti-CD45 ADCs (particularly an ADC comprising an anti-CD45 antibody conjugated via a protease cleavable linker to an IGN, e.g. DGN549), described herein are used as conditioning agents in human patients who are in need of a stem cell transplant, e.g., a hematopoietic stem cell (HSC) transplant. There is currently a need for compositions and methods for promoting the engraftment of stem cell transplants, e.g., exogenous hematopoietic stem cell grafts such that the multi-potency and hematopoietic functionality of these cells is preserved following transplantation. The compositions and methods disclosed herein provide a solution to this challenging problem.

Described herein are anti-CD45 ADCs that can be used to treat patients with conditions for which depletion of CD45+ cells is beneficial, including, but not limited to, leukemias and lymphomas, as well as patients hemoglobinopathy disorders, including sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S—C disease, Fanconi anemia, aplastic anemia, or Wiskott-Aldrich syndrome.

By targeting CD45 expressing cells with anti-CD45 ADCs described herein, generally both hematopoietic stem cells (HSCs) and leukocytes can be depleted (CD45 is a pan leukocyte marker). Thus, in certain embodiments, provided herein is a method for providing an immune reset in a subject in need thereof. For example, by administering an anti-CD45 ADC described herein to a patient having a disease associated with disease causing leukocytes, e.g., an autoimmune disease, the disease causing leukocytes can be eliminated (along with the HSCs) and the patient can then build a new immune system from subsequently transplanted HSCs.

An additional benefit of the CD45 specific ADCs described herein is that, as opposed to the non-targeted highly toxic chemotherapies commonly used in conditioning and treatment, red blood cells should be unaffected in the patient given that red blood cells do not generally express CD45.

Thus, disclosed herein are methods of treating a variety of disorders, such as diseases of a cell type in the hematopoietic lineage, cancers, autoimmune diseases, metabolic disorders, and stem cell disorders, among others. In certain embodiments, an anti-CD45 ADC described herein is used as a conditioning agent so as to deplete the CD45+ cells in the human subject prior to a stem cell transplant for treatment.

Thus, the compositions and methods described herein may (i) directly deplete a population of CD45 expressing cells that give rise to a pathology, such as a population of cancer cells (e.g., leukemia cells) and autoimmune cells (e.g., autoreactive T-cells), and/or (ii) deplete a population of endogenous hematopoietic stem cells so as to promote the engraftment of transplanted hematopoietic stem cells by providing a niche to which the transplanted cells may home. The foregoing activities can be achieved by administration of an anti-CD45 ADC capable of binding an endogenous disease-causing cell that expressed CD45 or a hematopoietic stem cell. In the case of direct treatment of a disease, this administration can cause a reduction in the quantity of the cells that give rise to the pathology of interest. In the case of preparing a patient for hematopoietic stem cell transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. In certain embodiments, ADCs, capable of binding CD45 can be administered to a patient to effect both of the foregoing activities. ADCs that bind CD45 antigen expressed by immune cells, e.g., hematopoietic stem cells, can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted hematopoietic stem cells.

In one embodiment an anti-CD45 ADC described herein is administered to a human patient for conditioning where the patient has acute myelogenous leukemia. A human patient may be administered a dose of about 0.1 mg/kg of an anti-CD45 ADC as a conditioning agent prior to a stem cell transplantation for treatment of acute myelogenous leukemia. A human patient may be administered a dose of about 0.11 mg/kg of an anti-CD45 ADC as a conditioning agent prior to a stem cell transplantation for treatment of acute myelogenous leukemia. A human patient may be administered a dose of about 0.15 mg/kg of an anti-CD45 ADC as a conditioning agent prior to a stem cell transplantation for treatment of acute myelogenous leukemia. A human patient may be administered a dose of about 0.17 mg/kg of an anti-CD45 ADC as a conditioning agent prior to a stem cell transplantation for treatment of acute myelogenous leukemia. A human patient may be administered a dose of about 0.2 mg/kg of an anti-CD45 ADC as a conditioning agent prior to a stem cell transplantation for treatment of acute myelogenous leukemia. A human patient may be administered a dose of about 0.22 mg/kg of an anti-CD45 ADC as a conditioning agent prior to a stem cell transplantation for treatment of acute myelogenous leukemia. A human patient may be administered a dose of about 0.3 mg/kg of an anti-CD45 ADC as a conditioning agent prior to a stem cell transplantation for treatment of acute myelogenous leukemia. A human patient may be administered a dose of about 0.37 mg/kg of an anti-CD45 ADC as a conditioning agent prior to a stem cell transplantation for treatment of acute myelogenous leukemia.

In one embodiment an anti-CD45 ADC described herein is administered to a human patient for conditioning where the patient has a myelodysplastic syndrome. A human patient may be administered a dose of about 0.1 mg/kg of an anti-CD45 ADC as a conditioning agent prior to a stem cell transplantation for treatment of a myelodysplastic syndrome. A human patient may be administered a dose of about 0.11 mg/kg of an anti-CD45 ADC as a conditioning agent prior to a stem cell transplantation for treatment a myelodysplastic syndrome. A human patient may be administered a dose of about 0.15 mg/kg of an anti-CD45 ADC as a conditioning agent prior to a stem cell transplantation for treatment of a myelodysplastic syndrome. A human patient may be administered a dose of about 0.17 mg/kg of an anti-CD45 ADC as a conditioning agent prior to a stem cell transplantation for treatment of a myelodysplastic syndrome. A human patient may be administered a dose of about 0.2 mg/kg of an anti-CD45 ADC as a conditioning agent prior to a stem cell transplantation for treatment of a myelodysplastic syndrome. A human patient may be administered a dose of about 0.22 mg/kg of an anti-CD45 ADC as a conditioning agent prior to a stem cell transplantation for treatment of a myelodysplastic syndrome. A human patient may be administered a dose of about 0.3 mg/kg of an anti-CD45 ADC as a conditioning agent prior to a stem cell transplantation for treatment of a myelodysplastic syndrome. A human patient may be administered a dose of about 0.37 mg/kg of an anti-CD45 ADC as a conditioning agent prior to a stem cell transplantation for treatment of a myelodysplastic syndrome.

In certain embodiments, and anti-CD45 ADC described herein is administered to a human patient having poor prognosis acute myeloid leukemia (AML) or high risk myelodysplastic syndrome (MDS).

Following conditioning with an anti-CD45 ADC, hematopoietic stem cell transplant therapy can be administered to a subject in need of treatment so as to populate or repopulate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population. The compositions and methods described herein can thus be used to treat a non-malignant hemoglobinopathy (e.g., a hemoglobinopathy selected from the group consisting of sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S—C disease, Fanconi anemia, aplastic anemia, and Wiskott-Aldrich syndrome).

Transplanted cells can be autologous or allogeneic. HSC transplants can be from bone marrow, peripheral blood or cord blood.

Additionally or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma. In some embodiments, the hematological cancer is a relapsed and/or refractory acute myeloid leukemia (AML). In some embodiments, the hematological cancer is a T cell lymphoma (e.g., a recurrent T cell lymphoma).

Additionally or alternatively, the compositions and methods described herein can be used to administer genetically-modified stem cells to a patient suffering from a condition that results from a defective gene (e.g., a mutation). For instance, the genome of living cells (e.g., stem cells) can be modified for therapeutic purposes. In particular, a therapeutic effect can be achieved by correcting a defective gene prior to engraftment of the genetically-modified stem cells back into the patient. In some embodiments, HSCs may be extracted from a patient suffering from a disorder caused by a defective gene and purified using methods known in the art. The isolated cells can be treated ex vivo using known methods in the art and its genome can be modified as desired e.g., edited to correct the defective target gene into a functional gene. The genetically-modified stem cells are subsequently administered back to the patient who has been conditioned using the compositions and methods described herein. In some embodiments, the stem cells may be allogeneic to the patient to whom they are administered.

Additionally or alternatively, the compositions and methods described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The antibodies, or antigen-binding fragments thereof, and conjugates described herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the compositions and methods described herein may be used to induce transplant tolerance in a solid organ transplant recipient (e.g., a kidney transplant, lung transplant, liver transplant, and heart transplant, among others). The compositions and methods described herein are well-suited for use in connection the induction of solid organ transplant tolerance, for instance, because a low percentage temporary or stable donor engraftment is sufficient to induce long-term tolerance of the transplanted organ.

In some embodiments, the transplant is allogeneic. In some embodiments, the transplant is autologous.

In some embodiments, the transplant is a bone marrow transplant, a peripheral blood transplant, or a cord blood transplant.

In some embodiments, the transplant includes hematopoietic cells (e.g., hematopoietic stem cells).

In some embodiments, the transplant includes gene-edited stem cells.

In any of the embodiments described herein, the transplant may be any solid organ or skin transplant. In some embodiments, the transplant is selected from the group consisting of kidney transplant, heart transplant, liver transplant, pancreas transplant, lung transplant, intestine transplant and skin transplant.

In addition, the compositions and methods described herein can be used to treat cancers directly, such as cancers characterized by cells that are CD45+. For instance, the compositions and methods described herein can be used to treat leukemia, such as in patients that exhibit CD45+ leukemic cells. By depleting CD45+ cancerous cells, such as leukemic cells, the compositions and methods described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma. In some embodiments, the cancer is a relapsed and/or refractory acute myeloid leukemia (AML). In some embodiments, the cancer is a recurrent T cell lymphoma.

In addition, the compositions and methods described herein can be used to treat autoimmune disorders. For instance, an antibody, or antigen-binding fragment thereof, can be administered to a subject, such as a human patient suffering from an autoimmune disorder, so as to kill a CD45+ immune cell. For example, a CD45+ immune cell may be an autoreactive lymphocyte, such as a T-cell that expresses a T-cell receptor that specifically binds, and mounts an immune response against, a self antigen. By depleting self-reactive, CD45+, the compositions and methods described herein can be used to treat autoimmune pathologies, such as those described below. Additionally or alternatively, the compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the compositions and methods described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

The ADCs described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate. In one embodiment, an anti-CD45 ADC described herein is intravenously administered to a human subject for conditioning prior to an HSC transplant.

The effective dose of an anti-CD45 ADC described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the antibody, or antigen-binding fragment thereof.

The dose of anti-CD45 ADC may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the antibody, or antigen-binding fragment thereof can be administered to the patient at a time that optimally promotes engraftment of the exogenous hematopoietic stem cells, for instance, from 1 hour to 1 week (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administration of the exogenous hematopoietic stem cell transplant.

In one embodiment, an anti-CD45 ADC described herein is administered about 10 days prior to the cell transplant.

In one embodiment, an HSC transplant if administered to a patient in need thereof 14 days following conditioning with an anti-CD45 ADC described herein. Alternatively, an HSC transplant is administered 14 days after a single dose administration of an anti-CD45 ADC described herein, but no later than 28 days following administration of the anti-CD45 ADC. Thus, in certain embodiments, there are 14 to 28 days between administration of the single dose (e.g., 0.066 mg/kg, 0.11 mg/kg, 0.15 mg/kg, 0.17 mg/kg, 0.22 mg/kg, 0.3 mg/kg, or 0.37 mg/kg) of the anti-CD45 ADC and delivery of the HSC transplant to the human patient in need thereof.

Using the methods disclosed herein, a physician of skill in the art can administer to a human patient in need of hematopoietic stem cell transplant therapy an ADC, capable of binding CD45 expressed by hematopoietic stem cells. In this fashion, a population of endogenous hematopoietic stem cells can be depleted prior to administration of an exogenous hematopoietic stem cell graft so as to promote engraftment of the hematopoietic stem cell graft. The antibody may be covalently conjugated to a toxin, such as an indolinobenzodiazepine, e.g., DGN-549. The anti-CD45 ADC can subsequently be administered to the patient, for example, by intravenous administration, prior to transplantation of exogenous hematopoietic stem cells (such as autologous, syngeneic, or allogeneic hematopoietic stem cells) or genetically-modified HSCs to the patient.

The anti-CD45 antibody, antigen-binding fragment thereof, or ADC can be administered in an amount sufficient to reduce the quantity of the target CD45 expressing cells. In one embodiment, the anti-CD45 ADC is administered at a dose of 0.1 mg/kg to 0.4 mg/kg, e.g., 0.1 mg/kg, 0.15 mg/kg, 0.2 mg/kg, 0.25 mg/kg, 0.3 mg/kg, 0.35 mg/kg, 0.4 mg/kg, or another dose described herein. For example, the anti-CD45 ADC can be administered in an amount sufficient to reduce the quantity of endogenous CD45+ cells in the bone marrow and/or in the peripheral blood by, for example, about 10%, 20%, 30%, 40%, 50%, 60%, 70%, 80%, 90%, 95%, or more. In some embodiments, the anti-CD45 antibody, antigen-binding fragment thereof, or ADC can be administered in an amount sufficient to reduce the quantity of endogenous hematopoietic stem cells, for example, by 10%, 20%, 30%, 40%, 50%, 60%, 70%, 80%, 90%, 95%, or more prior to hematopoietic stem cell transplant therapy. The reduction in hematopoietic stem cell count can be monitored using conventional techniques known in the art, such as by FACS analysis of cells expressing characteristic hematopoietic stem cell surface antigens in a blood sample withdrawn from the patient at varying intervals during conditioning therapy. For instance, a physician of skill in the art can withdraw a blood sample from the patient at various time points during conditioning therapy and determine the extent of endogenous hematopoietic stem cell reduction by conducting a FACS analysis to elucidate the relative concentrations of hematopoietic stem cells in the sample using antibodies that bind to hematopoietic stem cell marker antigens. According to some embodiments, when the concentration of hematopoietic stem cells has reached a minimum value in response to conditioning therapy with an anti-CD45 ADC, the physician may conclude the conditioning therapy, and may begin preparing the patient for hematopoietic stem cell transplant therapy.

The anti-CD45 ADC can be administered to the patient at a dose of 0.1 mg/kg to 0.4 mg/kg prior to administration of a hematopoietic stem cell transplant to the human patient. In one embodiment, the antibody, antigen-binding fragment thereof, or drug-antibody conjugate can be administered to the patient at a dosage of about 0.1 mg/kg to about 0.3 mg/kg. In one embodiment, the anti-CD45 ADC as described herein is administered to the patient at a dosage of about 0.15 mg/kg to about 0.3 mg/kg. In one embodiment, the anti-CD45 ADC as described herein is administered to the patient at a dosage of about 0.15 mg/kg to about 0.25 mg/kg. In one embodiment, the anti-CD45 ADC as described herein is administered to the patient at a dosage of about 0.2 mg/kg to about 0.3 mg/kg. In one embodiment, the anti-CD45 ADC as described herein is administered to the patient at a dosage of about 0.25 mg/kg to about 0.3 mg/kg. In some embodiments, the anti-CD45 ADC as described herein is administered to the patient at a dosage of about 0.066 mg/kg, 0.1 mg/kg, 0.11 mg/kg, 0.15 mg/kg, 0.17 mg/kg, 0.2 mg/kg, 0.22 mg/kg, 0.3 mg/kg, 0.37 mg/kg, or 0.4 mg/kg. In other embodiments, a dose of 0.5 mg/kg, 0.6 mg/kg, 0.7 mg/kg, 0.8 mg/kg, 0.9 mg/kg, or 1.0 mg/kg is administered.

In other embodiments, the anti-CD45 ADC as described herein is administered to the patient at a dosage of about 0.001 mg/kg to about 0.4 mg/kg, about 0.01 mg/kg to about 0.4 mg/kg, about 0.1 mg/kg to about 0.4 mg/kg, about 0.1 mg/kg to about 0.35 mg/kg, about 0.1 mg/kg to about 0.3 mg/kg, about 0.1 mg/kg to about 0.25, about 0.1 mg/kg to about 0.2 mg/kg, or about 0.1 mg/kg to about 0.15.

In certain embodiments, an anti-CD45 antibody, antigen-binding fragment thereof, or ADC described herein can be administered to the patient at a time that optimally promotes engraftment of the exogenous hematopoietic stem cells, for instance, from about 1 hour to about 1 week (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administration of the exogenous hematopoietic stem cell transplant.

The anti-CD45 ADC described herein may be used as a monotherapy conditioning agent. A monotherapy conditioning agent is an agent used in the absence of other myeloablative agents, e.g., busulfan. A human patient may be administered a dose of 0.1-0.4 mg/kg of an anti-CD45 ADC described herein as a monotherapy conditioning agent, where the anti-CD45 ADC alone conditions the patient and is not reliant on a combination of myeloablative agents to achieve conditioning prior to a transplant, e.g., an HSC transplant.

In one embodiment, an anti-CD45 ADC described herein is used as a monotherapy conditioning agent prior to an allogeneic or autologous hematopoietic stem cell (HSC) transplantation.

Following the conclusion of conditioning therapy, the patient may then receive an infusion (e.g., an intravenous infusion) of exogenous hematopoietic stem cells, such as from the same physician that performed the conditioning therapy or from a different physician. The physician may administer the patient an infusion of autologous, syngeneic, or allogeneic hematopoietic stem cells, for instance, at a dosage of from about 1×10³ to about 1×10⁹ hematopoietic stem cells/kg. The physician may monitor the engraftment of the hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes) following administration of the transplant. This analysis may be conducted, for example, from about 1 hour to about 6 months, or more, following hematopoietic stem cell transplant therapy (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). A finding that the concentration of hematopoietic stem cells or cells of the hematopoietic lineage has increased (e.g., by about 1%, 2%, 3%, 4%, 5%, 6%, 7%, 8%, 9%, 10%, 20%, 30%, 40%, 50%, 60%, 70%, 80%, 90%, 100%, 200%, 500%, or more) following the transplant therapy relative to the concentration of the corresponding cell type prior to transplant therapy provides one indication that treatment with the anti-CD45 ADC has successfully promoted engraftment of the transplanted hematopoietic stem cell graft. The foregoing may also be used in therapies relating to CD45 cell; depletion, e.g., HSC and immune cell depletion for treatment of an autoimmune disease, or for treatment of a hematological cancer.

Engraftment of hematopoietic stem cell transplants due to the administration of an anti-CD45 ADCs, can manifest in a variety of empirical measurements. For instance, engraftment of transplanted hematopoietic stem cells can be evaluated by assessing the quantity of competitive repopulating units (CRU) present within the bone marrow of a patient following administration of an anti-CD45 ADC, and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample. In some embodiments, the conditioning methods described herein are effective to achieve polyclonal engraftment.

The anti-CD45 ADC can, in certain embodiments, be administered to the patient in an aqueous solution containing one or more pharmaceutically acceptable excipients, such as a viscosity-modifying agent. The aqueous solution may be sterilized using techniques described herein or known in the art.

ADCs described herein can be administered to a patient (e.g., a human patient suffering from cancer, an autoimmune disease, or in need of hematopoietic stem cell transplant therapy) in a variety of dosage forms. For instance, antibodies, antigen-binding fragments thereof, or ADCs described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art.

A pharmaceutical formulation comprising an anti-CD45 ADC as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, or gelatin; hydrophilic polymers such as polyvinylpyrrolidone; amino acids such as glycine, glutamine, asparagine, arginine, or lysine; monosaccharides, disaccharides, and other carbohydrates including mannose, or dextrins; chelating agents such as EDTA; mannitol, trehalose or sorbitol; salt-forming counter-ions such as sodium; metal complexes (e.g. Zn-protein complexes); and/or non-ionic surfactants such as polyethylene glycol (PEG).

### Methods of Stem Cell Gene Therapy

Methods of the present disclosure include administering a population of genetically-modified stem cells to a patient suffering from a condition that results from a defective gene (e.g., a mutation). Generally, the present methods relate to stem cell gene therapy, in which the genome of living cells (e.g., stem cells) is modified for therapeutic purposes. In particular, a therapeutic effect can be achieved by correcting a defective gene (i.e., introducing a therapeutic edit), as described herein. By way of example, hematopoietic stem cell (HSCs) may be extracted from a patient suffering from a disorder caused by the defective gene (e.g., a sickle cell patient with a defective HBB gene) and purified by selecting for CD34 expressing cells (CD34+). In the case of sickle cell disease, the presence of a mutation in the β-hemoglobin gene (HBB) may result in a conformational change in the HbS protein and lead to sickle shaped red blood cells. In this instance, an example of a therapeutic edit is a genetic modification that corrects the mutation in HBB, thereby having a therapeutic effect, e.g., production of HbS protein having the correct conformation, normal red blood cell morphology, etc. The isolated cells can be treated ex vivo using known methods in the art, and its genome can be modified as desired, e.g., edited (therapeutic edit) to correct the defective target gene into a functional gene. Such modified stem cells comprising a therapeutic edit are subsequently administered back to the patient. The transplanted stem cells take root in the patient's bone marrow, replicating and creating cells that mature and create normally functioning protein, thereby resolving the problem.

As a further example, in some hemoglobinopathies, the β-hemoglobin produced is mutated, reduced, or absent, leading to an imbalance of α-hemoglobin: β-hemoglobin in the cell and therefore misregulated amounts of adult hemoglobin (HbA) in the subject. In this instance, a therapeutic edit may increase production of γ-hemoglobin (e.g., derepress HBG1 and/or HBG2 expression), resulting in increased levels of fetal hemoglobin (HbF).

Examples of therapeutic edits associated with hemoglobinopathies such as sickle cell disease and β-thalassemia are known in the art, for example in PCT Publication No. WO2017160890A1, WO2019118516A1, WO2016135558A2, WO2017182881A2, each of which is incorporated by reference in its entirety.

It will be evident to one of ordinary skill in the art how to assess the effect of a therapeutic edit and determine whether a genetic modification has a therapeutic effect (e.g., is a therapeutic edit).

In some embodiments, the therapeutic edit confers a change in the coding sequence of a protein thereby changing an amino acid in the protein product of the gene. In some embodiments, the therapeutic edit confers a change in a non-coding sequence of a gene, thereby altering the expression of the protein product without changing its sequence. In some embodiments, the therapeutic edit is located within a non-coding sequence of a gene. In some embodiments, the therapeutic edit is in a transcriptional control element operably linked to a gene associated a disease or disorder. It will be understood by those of skill in the art that transcriptional control elements include, but are not limited to, promoters, enhancers, operators, insulators, silencers and other cis- and trans-regulatory elements that control gene expression. In some embodiments, the therapeutic edit is in a portion of the gene and transcriptional control element. In some embodiments, the therapeutic edit is in a genomic region associated with the disease or disorder.

Methods of isolating stem cells from a source and further treatment of the cells ex vivo (e.g., expansion and genome modification) are well known and available in the art. In some embodiments, the stem cells are allogeneic to the mammal to which they are administered. In some embodiments, the stem cells are autologous to the mammal to which they are administered.

In some embodiments, the stem cells are isolated from bone marrow. In some embodiments, the stem cells are isolated from peripheral blood, e.g., mobilized peripheral blood. In some embodiments, the mobilized peripheral blood is isolated from a subject who has been administered a G-CSF. In some embodiments, the mobilized peripheral blood is isolated from a subject who has been administered a mobilization agent other than G-CSF, for example, Plerixafor® (AMD3100). In some embodiments, the stem cells are isolated from umbilical cord blood.

In some embodiments, the isolated stem cells comprise or consist of CD34+ cells. In some embodiments, the cells are substantially free of CD34− cells. In some embodiments, the cells comprise or consist of CD34+/CD90+ stem cells. In some embodiments, the cells comprise or consist of CD34+/CD90− cells. In some embodiments, the cells are a population comprising one or more of the cell types described above or described herein.

In some embodiments, any one or more of known genetically-modified stem cells can be used in the present methods, including, e.g., Strimvelis™ (autologous CD34+ enriched cell fraction that contains CD34+ cells transduced with retroviral vector that encodes for the human ADA cDNA sequence).

The genetically-modified HSC described herein may be used in genetically-modified stem cell therapy, or stem cell gene therapy, which refers to the in vitro gene editing (e.g., by CRISPR/Cas system or by viral transduction) of cells to form genetically-modified cells prior to introducing into a patient. Therefore, the genetically-modified stem cells described herein are used in methods of gene therapy because they contain the altered or corrected gene, and/or contain an exogenous gene. In particular, the genetically-modified stem cells described herein are useful in methods of gene therapy because all or most progeny from the modified stem cells will contain the altered or corrected gene. The modified hematopoietic cells can therefore be used for treatment of a mammalian subject, such as a human subject, suffering from a condition including but not limited to, inherited disorders, cancer, and certain viral infections.

As described herein, the genetically-modified stem cells are administered (i.e., transplanted) to a patient that has been conditioned using the ADCs and methods as described herein to ensure or improve engraftment.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

Studies in the examples below were performed using an anti-CD45 ADC comprising anti-CD45 Ab5 conjugated to DGN-549 via cysteines at amino acid 265 (EU numbering by Kabat) in the Fc region of the antibody. The linker conjugating the antibody to the toxin was a protease-cleavable linker (see linker described in FIG. 19).

### Example 1. Anti-CD45-ADC Depletes HSCs and Peripheral Cells In Vitro

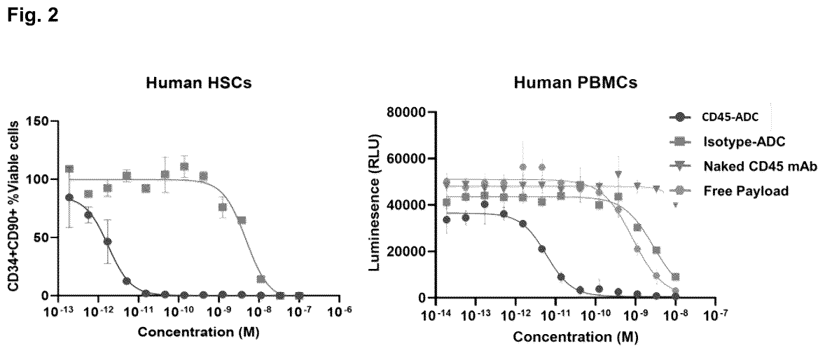

Primary human HSCs and peripheral blood mononuclear cells (PBMCs) from independent donors were treated with anti-CD45-ADC in vitro (FIG. 2). The anti-CD45-ADC exhibited an IC₉₀ of 6.6±5.9 pM in human stem cells, 5.9±1.2 pM in PBMCs, and 8.5±4.3 in an REH Leukemia cell line (Table 2)

TABLE 2
Cytotoxicity of CD45-ADC

| | Human HSCs | Human | REH Leukemia |
|---|---|---|---|
| | (CD34 + CD90+) | PBMCs | Cell Line |
| Cytotoxicity (IC90) | 6.6 ± 5.9 | 5.8 ± 1.2 | 8.5 ± 4.3 |
| pM | | | |

### Example 2. Anti-CD45-ADC Depletes HSCs and Immune Cells in Humanized Mice

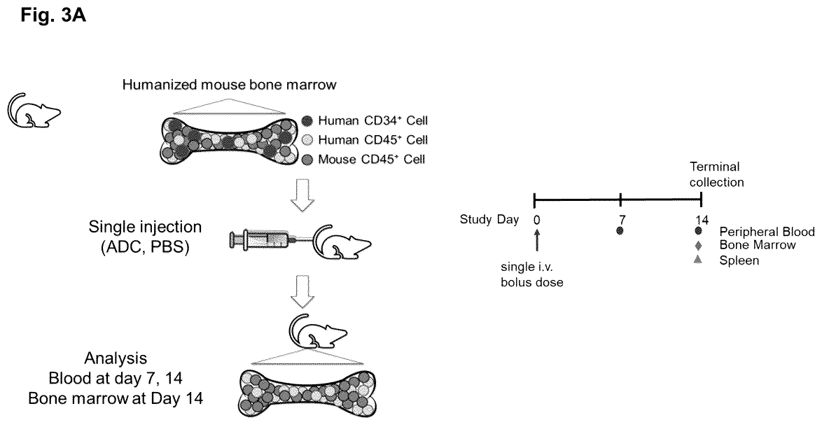

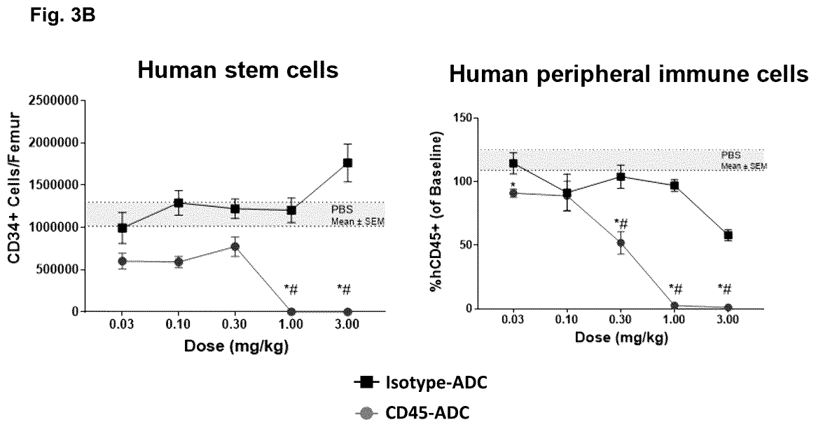

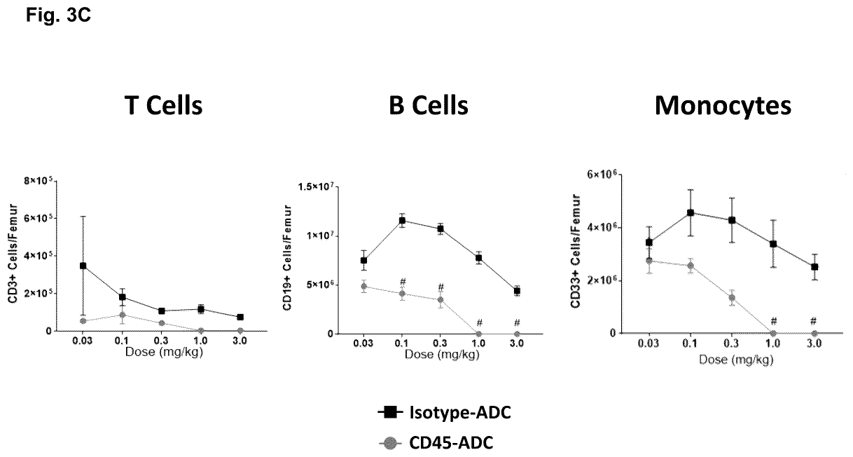

Humanized mice were treated with a single dose of an anti-CD45-ADC at a dose of 1 mg/kg. Peripheral blood samples were assessed at days 7 and 14 post-treatment and bone marrow was assessed at day 14 post-treatment (FIG. 3A). This treatment achieved >95% depletion of human HSCs and peripheral cells (FIG. 3B). Similarly, treatment achieved >95% depletion of immune cell types in bone marrow (FIG. 3C). A control non-targeting isotype-ADC had minimal effect on depletion (FIG. 3B-C).

In summary, a single dose of CD45-ADC depleted stem and immune cells in the bone marrow and peripheral blood of humanized NSG mice in a dose-dependent manner.

### Example 3. Anti-CD45-ADC Promotes Disease-Free Survival in PDX Mouse Models

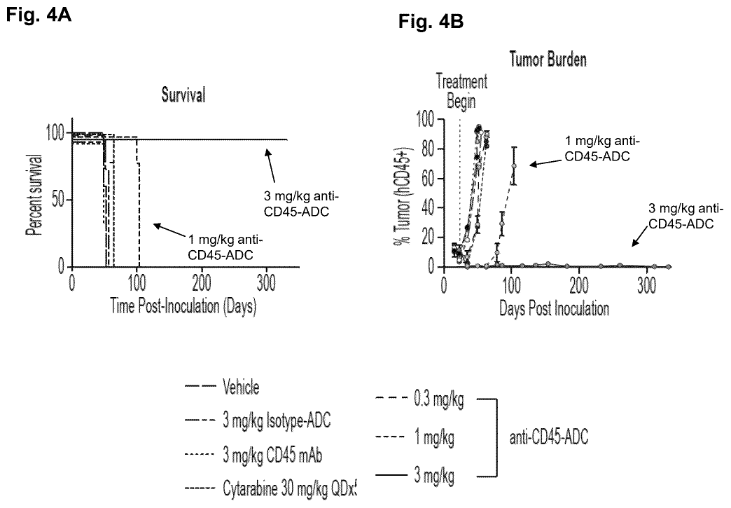

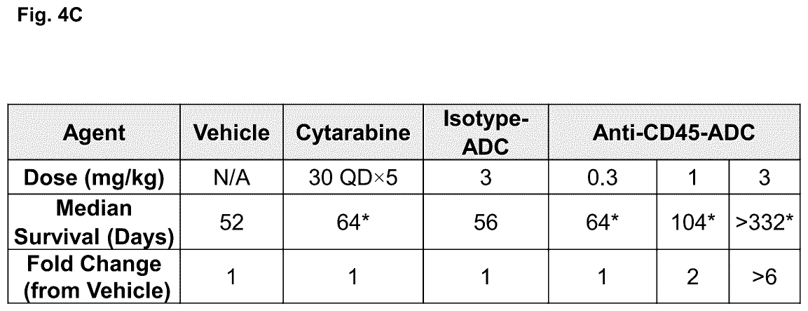

Anti-malignancy activity of an anti-CD45-ADC was assessed using a patient-derived xenograft (PDX) model of relapsed/refractory acute myeloid leukemia (AML). A single dose of anti-CD45-ADC (Ab5-DGN549) was administered to a group of mice when tumor levels reached ˜10% in peripheral blood, around day 24. Control groups received either PBS, isotype-ADC, CD45 mAb, or ARA-C at 30 mg/kg QDx5. Long-term, disease-free survival was monitored. Mice receiving the anti-CD45-ADC exhibited a median survival >332 days at the 3 mg/kg dose, as compared to control groups which had a median survival of 52 days (PBS), 56 days (Isotype-ADC at 3 mg/kg), 49 days (CD45 mAb at 3 mg/kg), and 64 days (ARA-C) (FIG. 4A-C).

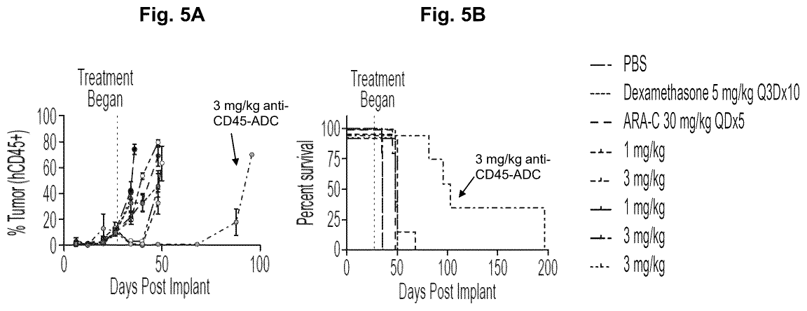

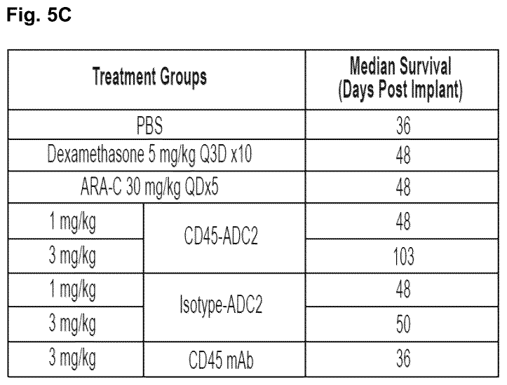

Anti-CD45-ADC was further tested in a PDX model of recurrent angioimmunoblastic T-cell lymphoma. A single dose of anti-CD45-ADC (Ab5-DGN549) was administered to a group of mice when tumor levels reached ˜10% in peripheral blood, around day 27. Control groups received either PBS, isotype-ADC, CD45 mAb, ARA-C at 30 mg/kg QDx5, or Dexamethasone at 5 mg/kg Q3Dx10. Long-term disease survival was monitored. Mice receiving the anti-CD45-ADC exhibited a median survival of 103 days at the 3 mg/kg dose, as compared to control groups which had a median survival of 36 days (PBS), 50 days (Isotype-ADC at 3 mg/kg), 36 days (CD45 mAb at 3 mg/kg), and 48 days (both ARA-C and Dexamethasone) (FIG. 5A-C).

In summary, a single dose of CD45-ADC extended median survival in a R/R AML PDX model and a recurrent T-cell lymphoma PDX model.

### Example 4. Anti-CD45-ADC Depletes HSC and Immune Cells in Non-Human Primates

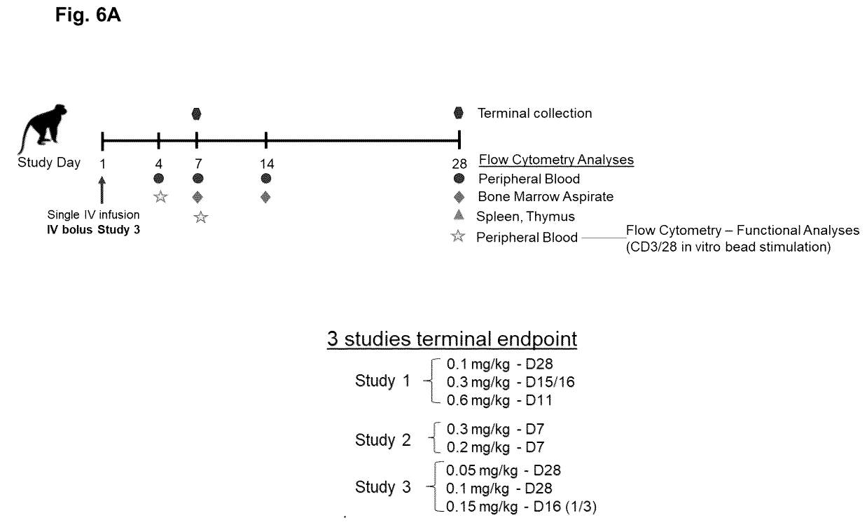

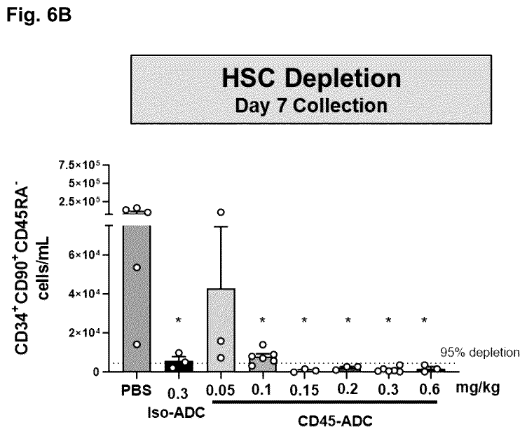

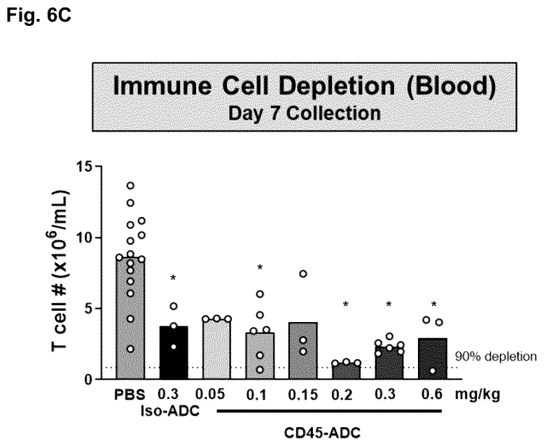

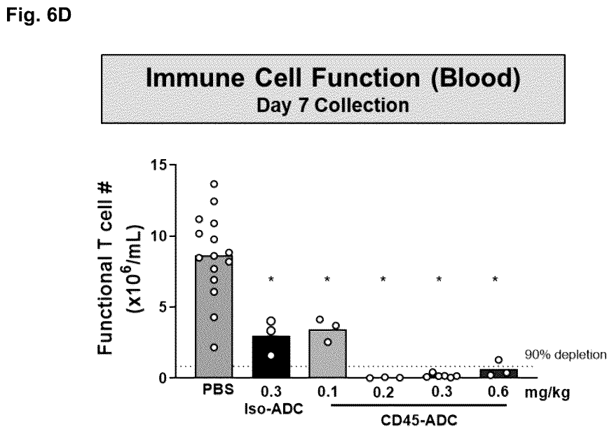

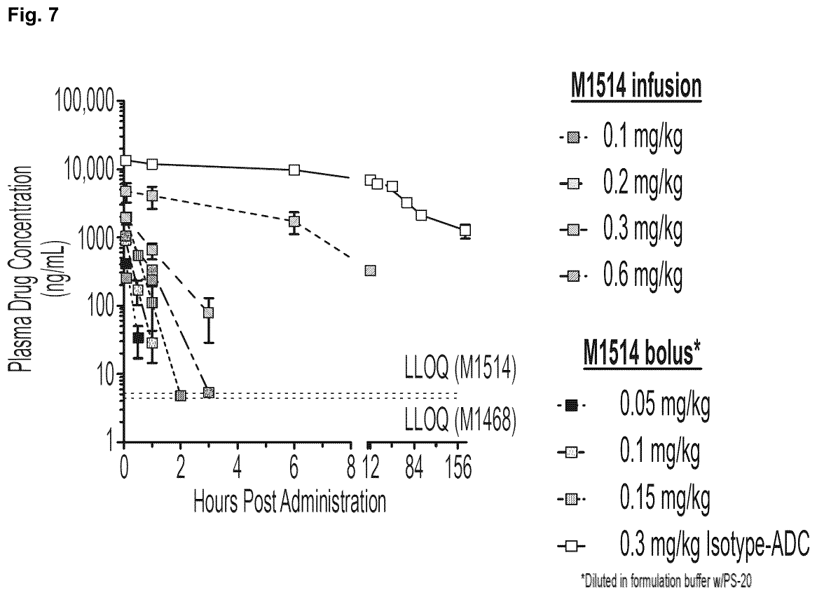

Cynomolgus monkeys were treated with a single dose of anti-CD45-ADC (Ab5-DGN549) at various doses ranging from 0.05 to 0.6 mg/kg. Control groups received either PBS or isotype-ADC. Samples were collected at days 4, 7, 14, and 28 for flow cytometry analysis (FIG. 6A). At 7 days post-administration, monkeys receiving anti-CD45-ADC at concentrations >0.15 mg/kg exhibited greater than 95% depletion of HSCs (FIG. 6B). Immune cell depletion across treatment groups is shown in FIG. 6C. Immune cell function in peripheral blood similarly showed a >90% depletion at doses >0.2 mg/kg (FIG. 6D). PK analysis of anti-CD45-ADC administered as either an infusion or a bolus demonstrated rapid in vivo clearance at various doses irrespective of administration route in cynomologus monkeys (FIG. 7).

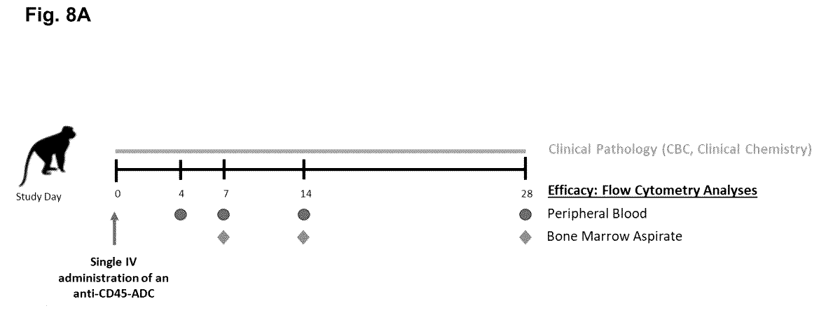

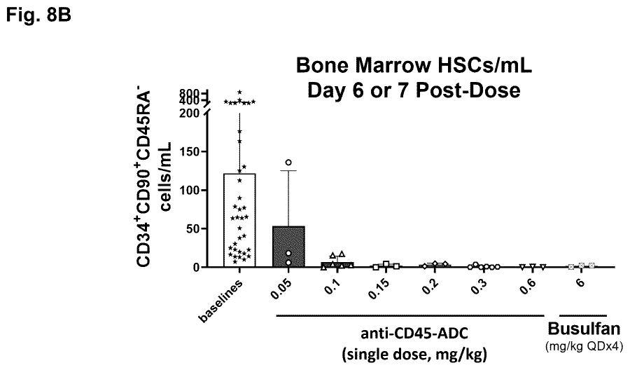

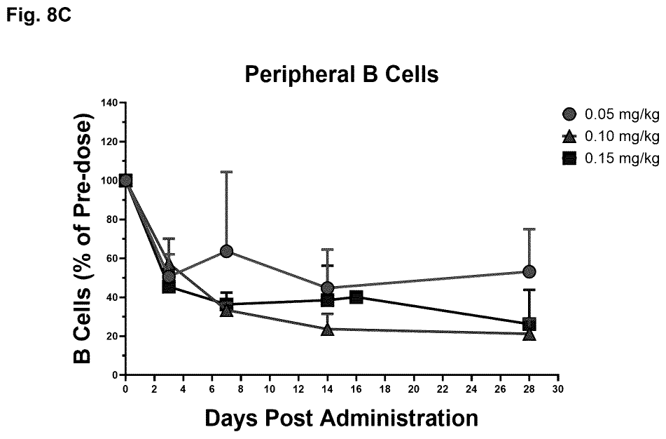

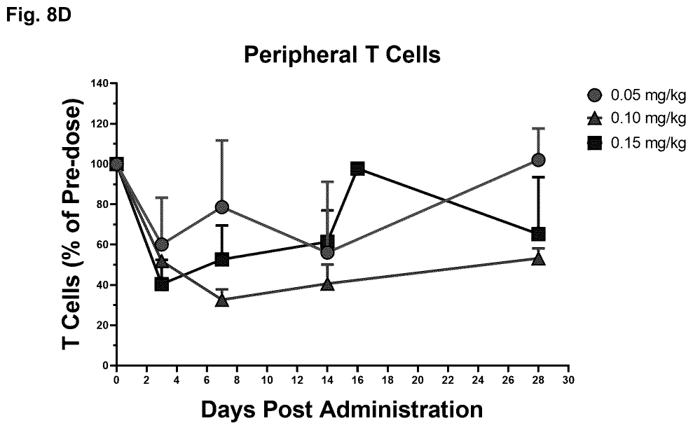

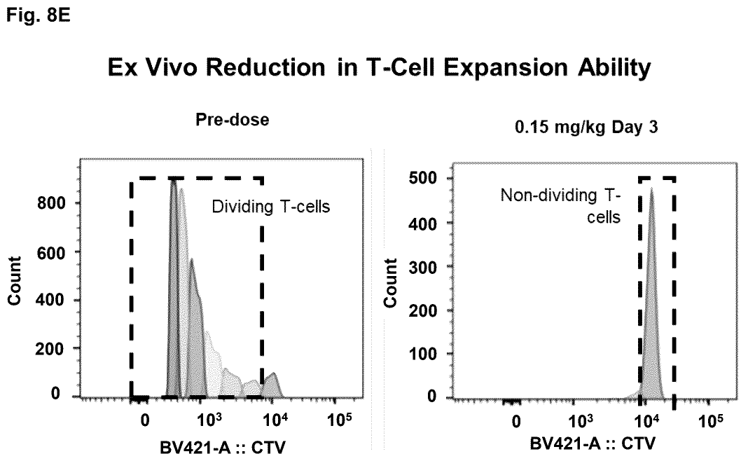

An additional experiment using Cynomolgus monkeys was performed, with an identical setup as described above (FIG. 8A). The anti-CD45-ADC was administered as a single dose at concentrations ranging from 0.05 to 0.6 mg/kg. Busulfan (administered at 6 mg/kg, QDx4) was utilized as a control. All doses tested depleted HSCs in bone marrow at days 6 or 7 post-dose relative to baseline. The anti-CD45-ADC at doses >0.1 mg/kg resulted in comparable reduction of HSCs as compared to the myeloablative Busulfan (FIG. 8B). The anti-CD45-ADC resulted in a reduction of peripheral B cells following administration, with levels of peripheral B cells staying at approximately consistent levels after ˜day 14 post-dose (FIG. 8C). While peripheral T cells were initially reduced following administration of the anti-CD45-ADC, these levels recovered over time. Monkeys treated with the lowest dose tested (0.05 mg/kg) had their peripheral T cells return to pre-dose levels (FIG. 8D). The anti-CD45-ADC was further shown to reduce the ability for T cells to divide ex vivo on day 3 (FIG. 8E).

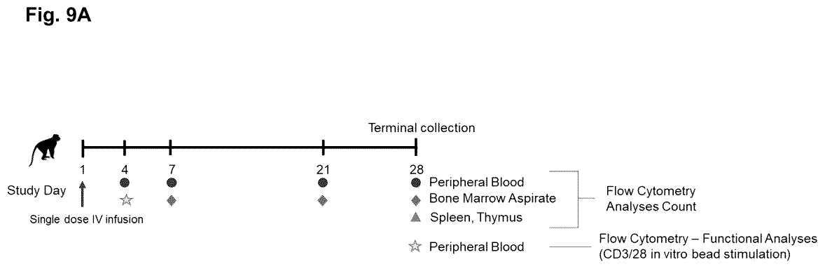

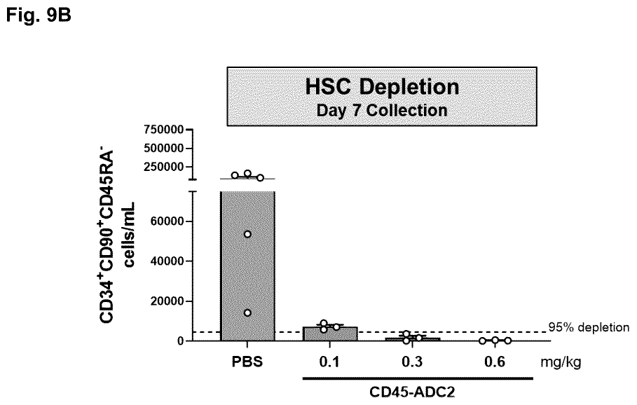

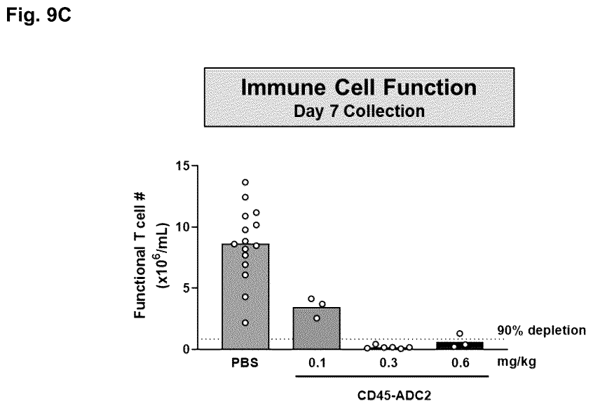

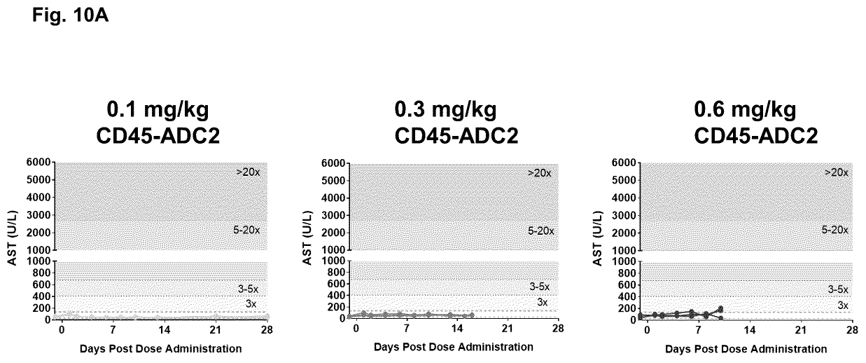

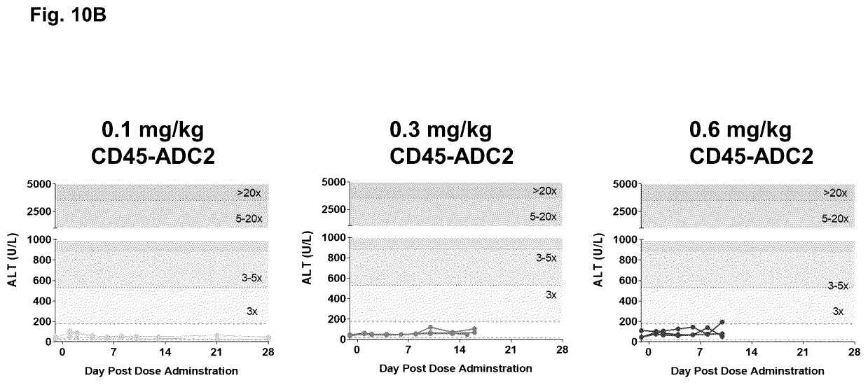

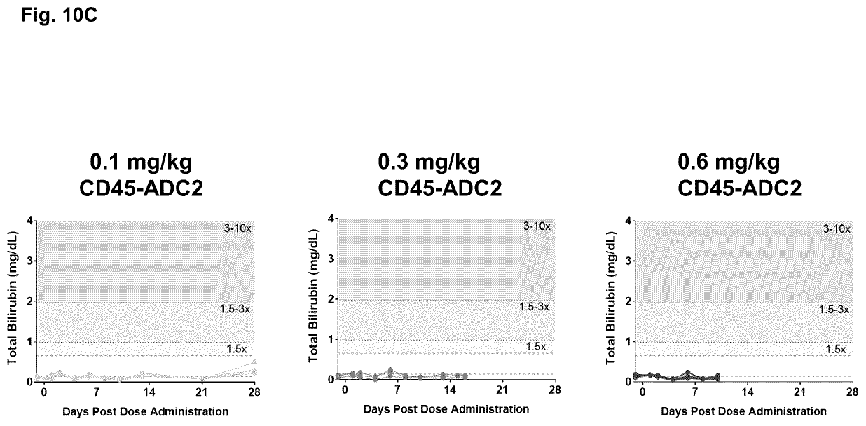

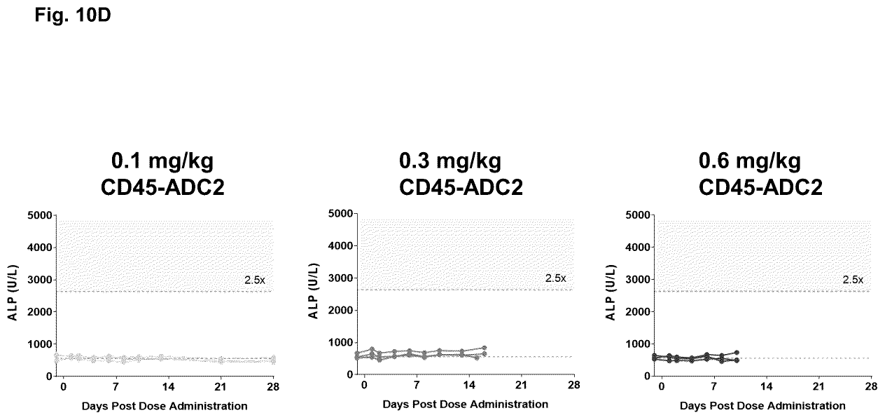

Rhesus macaques were treated with a single dose of anti-CD45-ADC at 0.1, 0.3, or 0.6 mg/kg. Control groups received PBS. Samples were collected at days 4, 7, 21, and 28 for flow cytometry analysis (FIG. 9A). At 7 days post-administration, macaques receiving anti-CD45-ADC at concentrations >0.3 mg/kg exhibited greater than 95% depletion of HSCs (FIG. 9B). Immune cell function in peripheral blood similarly showed a >90% depletion at doses >0.3 mg/kg (FIG. 9B). Clinical pathology was further assessed in rhesus macaques treated with a single IV infusion of anti-CD45-ADC at 0.1, 0.3, and 0.6 mg/kg. Macaques showed no changes in AST (FIG. 10A), ALT (FIG. 10B), TBIL (FIG. 10C), ALP (FIG. 10D) at any dose tested.

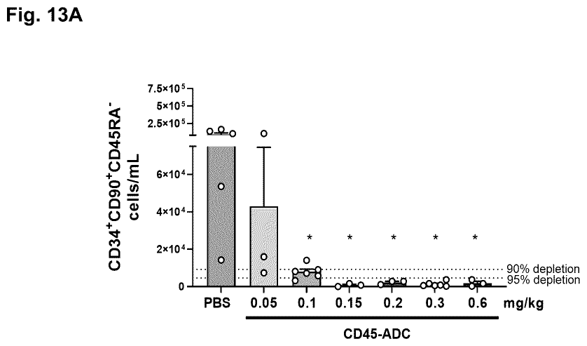

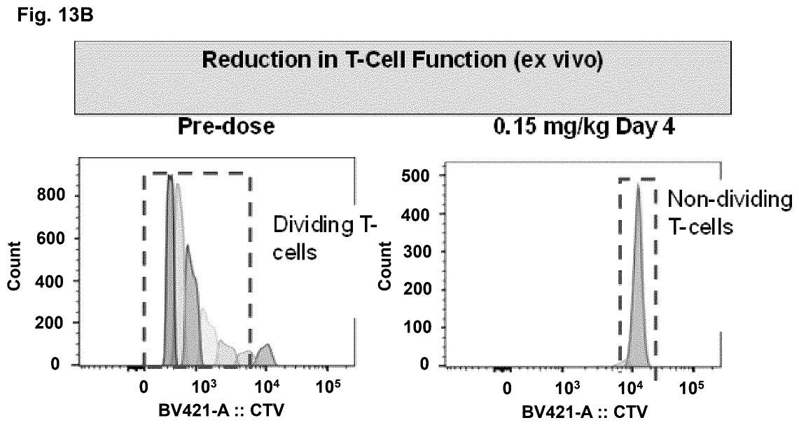

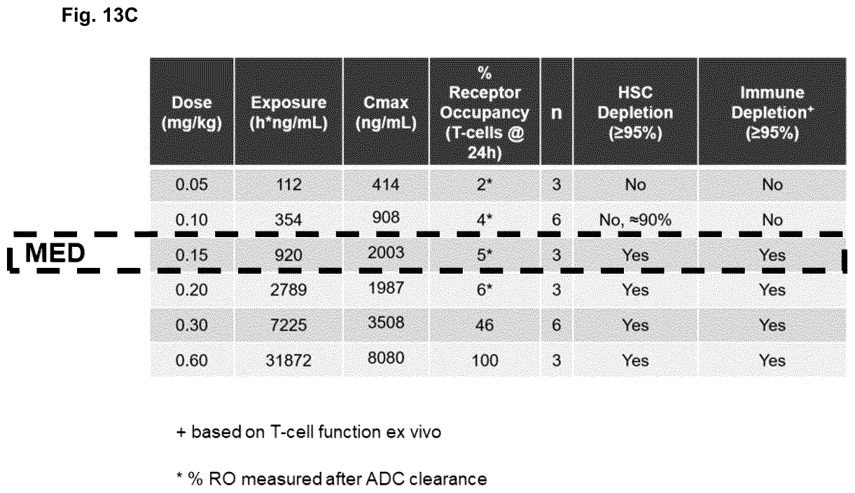

As shown in FIGS. 13A-13C, the minimum efficacious dose (MED) for NHPs treated with an anti-CD45 ADC, which resulted in >95% depletion of HSCs and immune function, was determined to be 0.15 mg/kg. 0.15 mg/kg CD45-DGN549 with AUC of 920 h*ng/mL achieves 99% HSC depletion and 97% reduction in functional T-cells (measured by inability to divide ex vivo). Low receptor occupancy was observed at MED (˜5% RO).

### Example 5. Anti-CD45-ADC Enables Engraftment of Gene-Modified Cells in Non-Human Primates

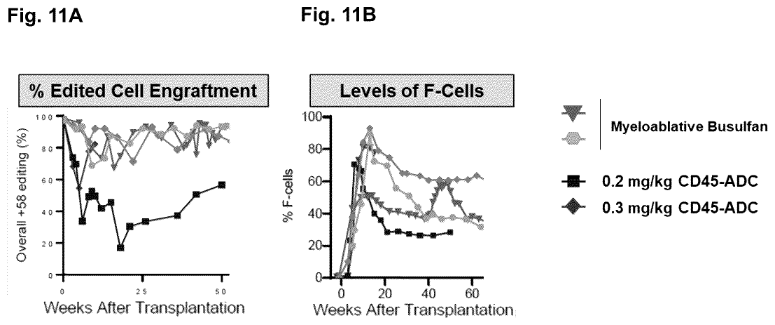

Rhesus macaques were treated with a single dose of anti-CD45-ADC (Ab5-DGN549) at either 0.2 or 0.3 mg/kg followed by engraftment of erythroid-specific BCL11A enhancer-edited CD34+ cells. Conditioning with the anti-CD45-ADC at 0.2 mg/kg resulted in ˜60% engraftment of the edited cells at week 50, while the 0.3 mg/kg dose resulted in ˜85% engraftment by week 10 (FIG. 11A). Both doses resulted in robust induction of levels of F-cells (FIG. 11B). Engraftment levels following conditioning at the 0.3 mg/kg dose were comparable to that of the myeloablative Busulfan (FIG. 11A).

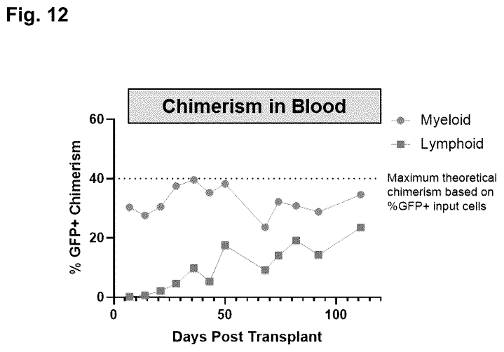

Using an immunogenic GFP+ model, chimerism in blood was assessed following conditioning with anti-CD45-ADC. At day 111, macaques exhibited ˜35% myeloid chimerism and ˜24% lymphoid chimerism, as compared to the ˜40% theoretical maximum chimerism based on transplant input (FIG. 12). Engraftment was performed using gene modified HSCs that contained tracer DNA in order to study the lineage of the cells.

### Example 6. Anti-CD45 Antibody-Drug Conjugate Conditioning Effectively Enables

### Engraftment of Gene Modified Cells in Non-Human Primates

To enable simultaneous HSC and immune cell depletion for HSC transplantation (HSCT) conditioning, an ADC targeting CD45 was developed, which targets a hematopoietic-specific receptor expressed broadly on HSCs and most mature immune cells. The ADC was engineered to enable rapid in vivo clearance prior to HSCT. The anti-CD45-ADC enabled potent in vitro killing of primary human HSCs as described above in Table 2.

In humanized mice, a single dose of anti-CD45-ADC (1 mg/kg, n=5/group) achieved >95% depletion of human HSCs and immune cells in bone marrow (p<0.05) and peripheral blood (p<0.05). A matched dose of non-targeting isotype-ADC had minimal effect. The anti-malignancy activity was assessed in a patient-derived xenograft (PDX) model of relapsed/refractory acute myeloid leukemia (AML). A single dose of anti-CD45-ADC (Ab5-DGN549) resulted in long-term disease-free survival in all animals (median >330 days) while control groups (vehicle, isotype-ADC, or standard of care (SOC)) had a median survival of only 52-64 days (p<0.005, n=5-8 mice/group).

In cynomolgus macaques, a single dose of >0.15 mg/kg anti-CD45-ADC (Ab5-DGN549) resulted in ˜99% bone marrow HSC depletion and broad peripheral immune depletion (n=3-6 animals/dose group) 7 days post administration. Pharmacokinetic analysis demonstrated that the anti-CD45-ADC had a rapid in vivo clearance (t_{1/2}<1.2 hour at 0.2 mg/kg) profile suitable for transplant.

For transplant studies, rhesus macaque CD34+ HSPCs were electroporated with 3×NLS SpCas9 protein and two RNAs targeting the +55 and +58 BCL11A erythroid enhancers. The edited cells showed editing of 93.15-98.65%, with γ-globin expression of 82-89% following erythroid differentiation compared to controls with 10-31% γ-globin (data not shown).

Conditioning and transplant in rhesus macaques were then explored in a gene therapy hemoglobinopathy model where a single dose of 0.2 or 0.3 mg/kg anti-CD45-ADC enabled efficient engraftment of erythroid-specific BCL11A enhancer-edited CD34+ cells, with 60-80% edited cell engraftment and robust hemoglobin F (HbF) induction as measured by HbF-positive cells. The engraftment level following 0.3 mg/kg anti-CD45-ADC conditioning was comparable to myeloablative Busulfan.

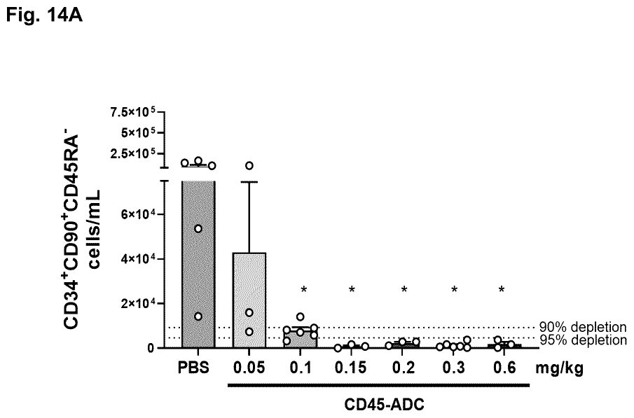

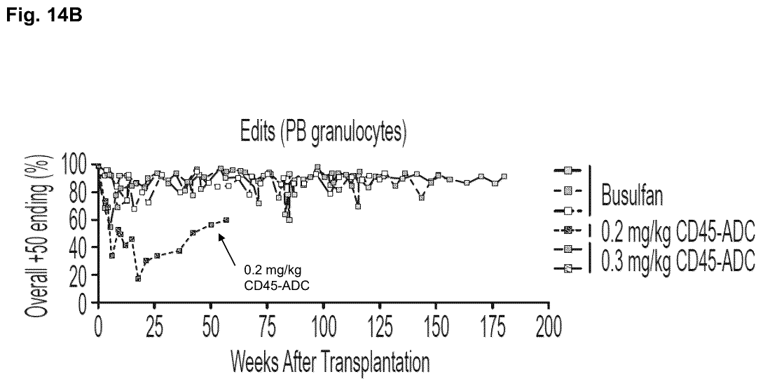

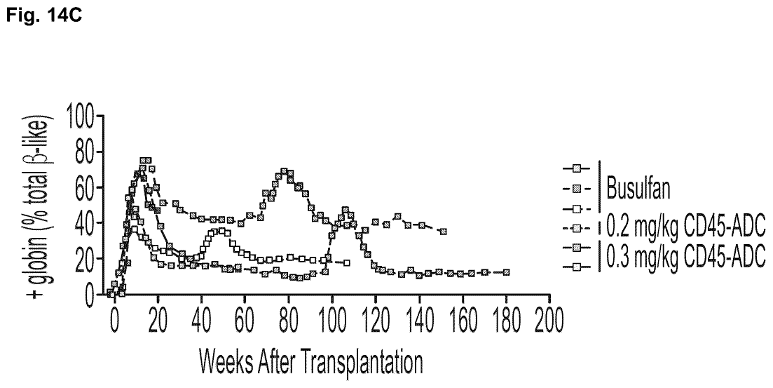

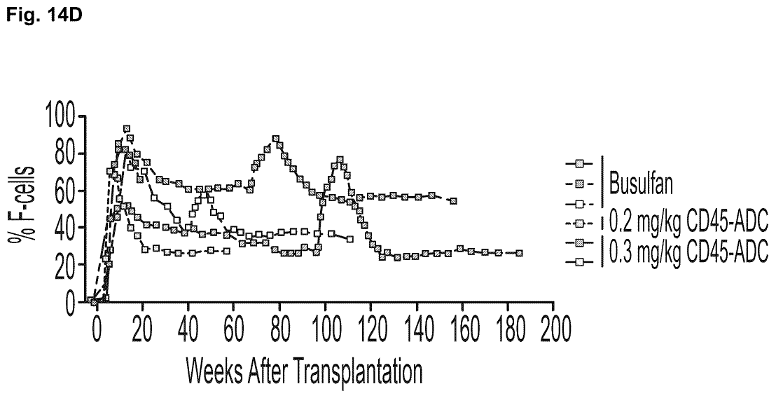

As shown in FIG. 14, anti-CD45-ADC demonstrated robust depletion of bone marrow HSCs in cynomolgus macaques (FIG. 14A) and allowed engraftment of erythroid-specific BCL11A enhancer-edited CD34+ cells (FIG. 14B). Engrafted cells showed robust HbF induction as measured by HbF positive cells (FIGS. 14C, 14D).

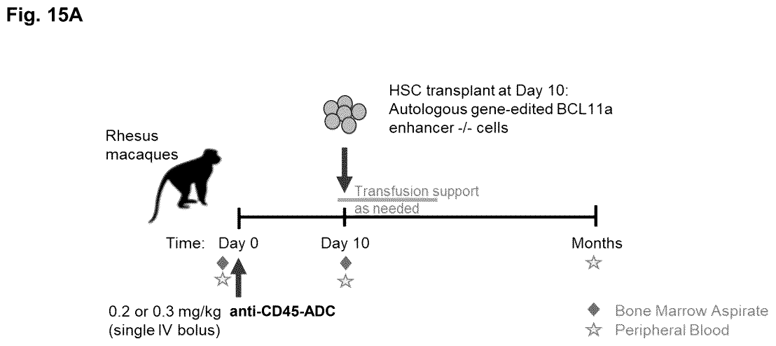

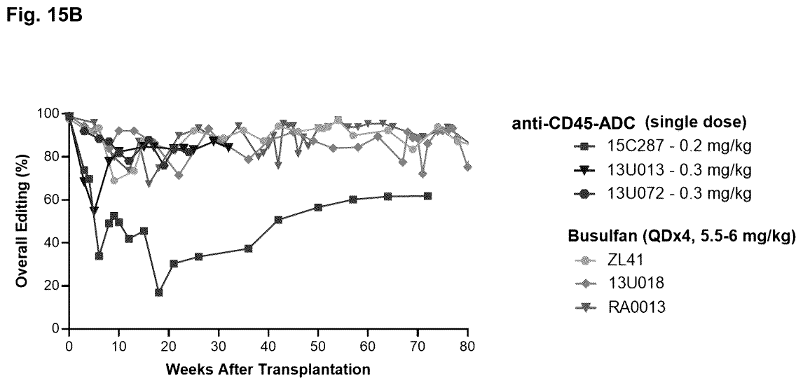

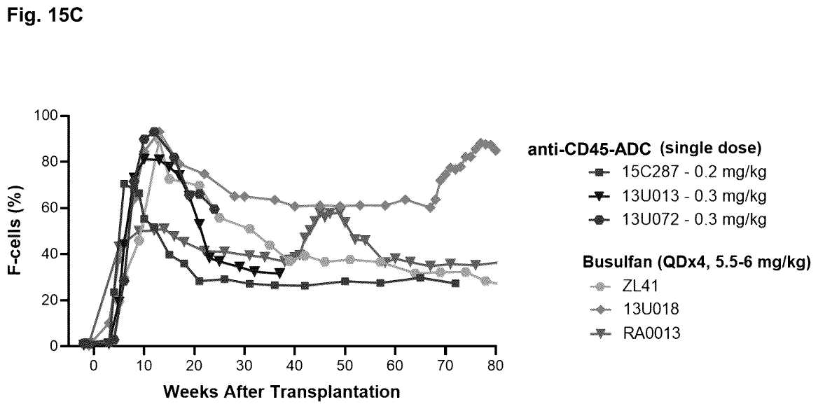

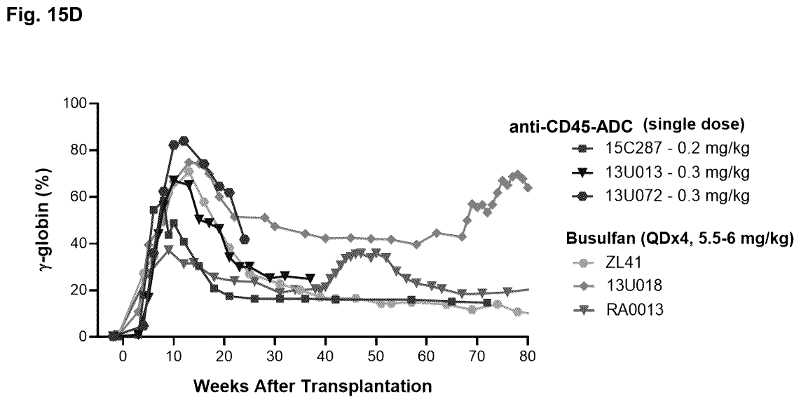

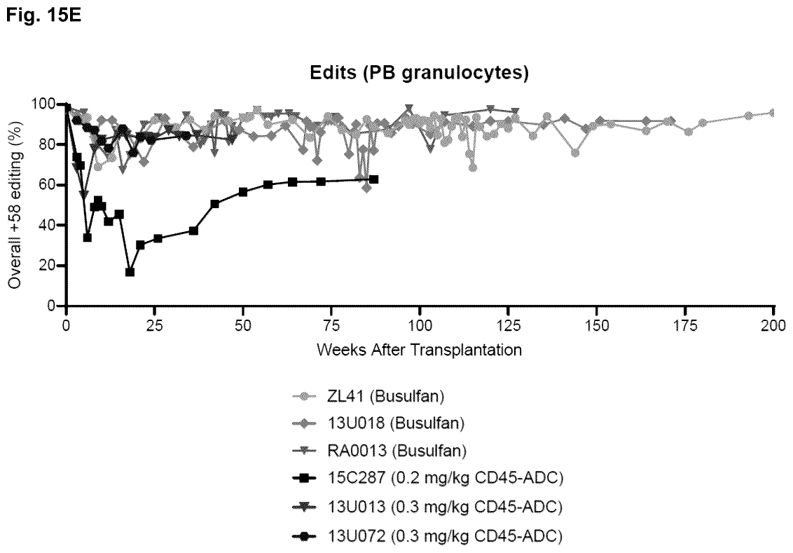

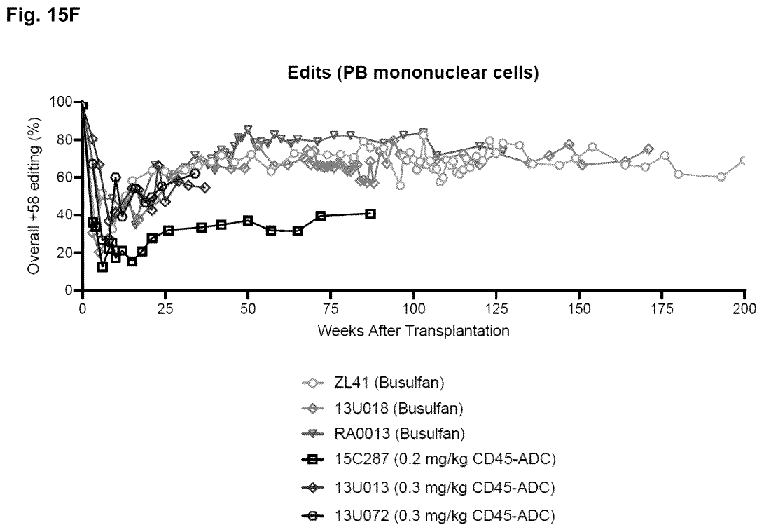

A similar experiment was performed in Rhesus macaques. Rhesus macaques were treated with either 0.2 or 0.3 mg/kg of an anti-CD45-ADC as a single IV bolus. At day 10, the macaques underwent an HSC transplant with autologous gene-edited BCL11a enhancer −/− cells, and were monitored over time (FIG. 15A). A cohort of macaques were treated with Busulfan (5.5-6 mg/kg QDx4) as a control.

The anti-CD45-ADC enabled engraftment of edited cells at all doses tested, with the 0.3 mg/kg dose providing comparable results as to the myeloablative Busulfan in both peripheral blood granulocytes (FIGS. 15B and 15E) and peripheral blood mononuclear cells (FIG. 15F). Macaques exhibited robust fetal hemoglobin induction following treatment with the anti-CD45-ADC as measured by both the percentage of F-cells (FIG. 15C) and γ-globin (FIG. 15D).

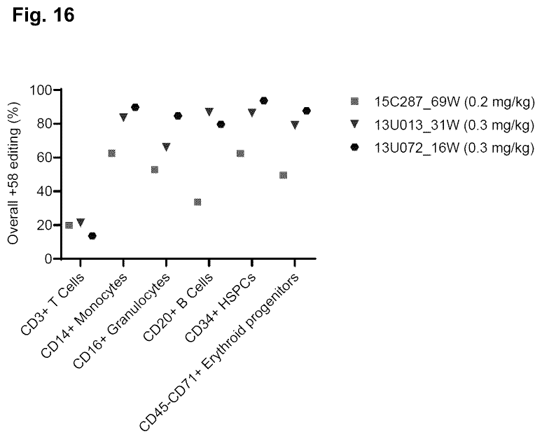

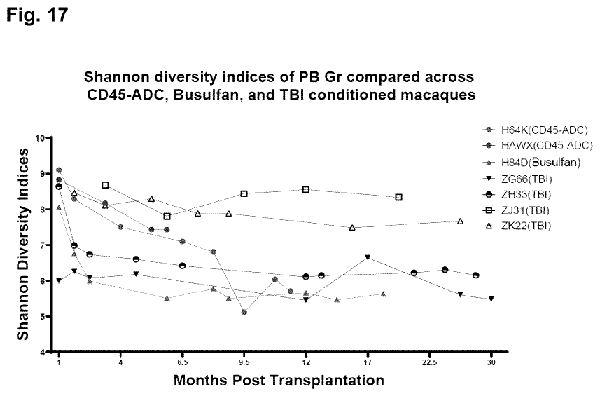

Indel frequencies in bone marrow subsets for each treatment condition are shown in FIG. 16. At 0.2 mg/kg anti-CD45 ADC (n=1), editing reached 62% of target alleles among short-lived granulocytes, reflecting the HSC compartment, with a follow up of 72 weeks to date. At 0.3 mg/kg anti-CD45 ADC (n=2), granulocyte editing reached 82-84% and remained stable through 24 and 32 weeks to date. γ-globin expression and F-cell levels reached 67-82% and 80-93% in these animals respectively, at week 10-13, comparable to control animals receiving dual edited cells after conditioning with myeloablative busulfan at 5.5 mg/kg×4 days (n=3). Editing in other lineages including monocytes, NK cells, B cells, erythroid precursors and CD34+ HPSCs ranged from 79-93% for the animals given 0.3 mg/kg anti-CD45 ADC and 34-63% for the animal given 0.2 mg/kg anti-CD45 ADC. At 16-69 week post-transplantation, T cell editing was 14-21% following anti-CD45 ADC conditioning (8-31% for busulfan treated animals at 25-34 weeks). Two additional rhesus macaques were conditioned with 0.2 mg/kg anti-CD45 ADC and infused with autologous HSPCs transduced with a barcoded lentiviral vector. At four to eight months after infusion, the animals were highly polyclonal, similar to TBI or busulfan conditioned animals (FIG. 17).

In summary, anti-CD45 ADC-based conditioning is myeloablative and achieved polyclonal early engraftment at levels equivalent to TBI or myeloablative busulfan. This approach allowed robust engraftment with BCL11A enhancer edited HSCs and achieved potentially therapeutic levels of HbF.

### CONCLUSION

Targeting CD45-expressing hematopoietic cells with a novel anti-CD45-ADC potently depleted HSCs and immune cells in vitro and in vivo, with rapid clearance, and enabled robust gene-modified cell engraftment in a clinically relevant NHP transplant model. Anti-CD45-ADC also significantly extended survival in a patient-derived xenograft model of AML refractory to SOC. Overall, targeted conditioning using anti-CD45-ADC could improve the risk-benefit profile of HSCT, expanding the patient population able to receive these potentially curative therapies.

Sequences referenced throughout this disclosure are provided in Table 3.

TABLE 3
SEQUENCE TABLE

| SEQ ID | | |
|---|---|---|
| NO | Description | Sequence |
| 1-40 | Sequences described in sequence listing | |
| 41 | Ab5 Heavy Chain | EVQLVESGGGLVQPGGSLRLSCAASGFTFEAYS |
| | Variable Region | MNWVRQAPGKGLEWVSYISLSGATIHYADSVKG |
| | (CDRs bolded) | RFTISRDNAKNSLYLQMNSLRAEDTAVYYCARG |
| | | GQYYYTSSDYGEVAFDIWGQGTMVTVSS |
| 42 | Ab5-HC CDR1 | FTFEAYSMN |
| 43 | Ab5-HC CDR2 | YISLSGATIHYADSVKG |
| 44 | Ab5-HC CDR3 | ARGGQYYYTSSDYGEVAFDI |
| 45 | Ab5 Light Chain | DIVMTQSPLSLPVTPGEPASISCRSSQSLVSSGY |
| | Variable Region | NYLDWYLQKPGQSPQLLIYFGSSRASGVPDRFS |
| | (CDRs bolded) | GSGSGTDFTLKISRVEAEDVGVYYCMQRRRTPW |
| | | SFGGGTKVEIK |
| 46 | Ab5-LC CDR1 | RSSQSLVSSGYNYLD |
| 47 | Ab5-LC CDR2 | FGSSRAS |
| 48 | Ab5-LC CDR3 | MQRRRTPWS |
| 49 | Ab5 Heavy Chain | EVQLVESGGGLVQPGGSLRLSCAASGFTFEAYS |
| | (CDRs in bold; | MNWVRQAPGKGLEWVSYISLSGATIHYADSVKG |
| | Constant region | RFTISRDNAKNSLYLQMNSLRAEDTAVYYCARG |
| | underlined; | GQYYYTSSDYGEVAFDIWGQGTMVTVSSASTKG |
| | D265C.LALA.H435A) | PSVFPLAPSSKSTSGGTAALGCLVKDYFPEPVTV |
| | | SWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTP |
| | | EVTCVVVCVSHEDPEVKFNWYVDGVEVHNAKTK |
| | | PREEQYNSTYRVVSVLTVLHQDWLNGKEYKCKV |
| | | SNKALPAPIEKTISKAKGQPREPQVYTLPPSRDEL |
| | | TKNQVSLTCLVKGFYPSDIAVEWESNGQPENNY |
| | | KTTPPVLDSDGSFFLYSKLTVDKSRWQQGNVFS |
| | | CSVMHEALHNAYTQKSLSLSPGK |
| 50 | Ab5 Light Chain | DIVMTQSPLSLPVTPGEPASISCRSSQSLVSSGY |
| | (CDRs in bold; | NYLDWYLQKPGQSPQLLIYFGSSRASGVPDRFS |
| | Constant region | GSGSGTDFTLKISRVEAEDVGVYYCMQRRRTPW |
| | underlined) | SFGGGTKVEIKRTVAAPSVFIFPPSDEQLKSGTA |
| | | SVVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| 51-101 | Sequences described in sequence listing | |
| 102 | IgG Heavy chain | ASTKGPSVFPLAPSSKSTSGGTAALGCLVK |
| | constant region of | DYFPEPVTVSWNSGALTSGVHTFPAVLQSS |
| | WT | GLYSLSSVVTVPSSSLGTQTYICNVNHKPSN |
| | | TKVDKKVEPKSCDKTHTCPPCPAPELLGGP |
| | | SVFLFPPKPKDTLMISRTPEVTCVVVDVSHE |
| | | DPEVKFNWYVDGVEVHNAKTKPREEQYNS |
| | | TYRVVSVLTVLHQDWLNGKEYKCKVSNKAL |
| | | PAPIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 103 | IgG Heavy chain | ASTKGPSVFPLAPSSKSTSGGTAALGCLVK |
| | constant region | DYFPEPVTVSWNSGALTSGVHTFPAVLQSS |
| | (D265C)* | GLYSLSSVVTVPSSSLGTQTYICNVNHKPSN |
| | | TKVDKKVEPKSCDKTHTCPPCPAPELLGGP |
| | | SVFLFPPKPKDTLMISRTPEVTCVVVCVSHE |
| | | DPEVKFNWYVDGVEVHNAKTKPREEQYNS |
| | | TYRVVSVLTVLHQDWLNGKEYKCKVSNKAL |
| | | PAPIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 104 | IgG Heavy chain | ASTKGPSVFPLAPSSKSTSGGTAALGCLVK |
| | constant region | DYFPEPVTVSWNSGALTSGVHTFPAVLQSS |
| | (L234A/L235A/ | GLYSLSSVVTVPSSSLGTQTYICNVNHKPSN |
| | D265C)* | TKVDKKVEPKSCDKTHTCPPCPAPEAAGGP |
| | | SVFLFPPKPKDTLMISRTPEVTCVVVCVSHE |
| | | DPEVKFNWYVDGVEVHNAKTKPREEQYNS |
| | | TYRVVSVLTVLHQDWLNGKEYKCKVSNKAL |
| | | PAPIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 105 | IgG Heavy chain | ASTKGPSVFPLAPSSKSTSGGTAALGCLVK |
| | constant region | DYFPEPVTVSWNSGALTSGVHTFPAVLQSS |
| | (H435A/D265C)* | GLYSLSSVVTVPSSSLGTQTYICNVNHKPSN |
| | | TKVDKKVEPKSCDKTHTCPPCPAPELLGGP |
| | | SVFLFPPKPKDTLMISRTPEVTCVVVCVSHE |
| | | DPEVKFNWYVDGVEVHNAKTKPREEQYNS |
| | | TYRVVSVLTVLHQDWLNGKEYKCKVSNKAL |
| | | PAPIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNAYTQKSLSLSPGK |
| 106 | IgG Heavy chain | ASTKGPSVFPLAPSSKSTSGGTAALGCLVK |
| | constant region | DYFPEPVTVSWNSGALTSGVHTFPAVLQSS |
| | (L234A/L235A/ | GLYSLSSVVTVPSSSLGTQTYICNVNHKPSN |
| | H435A/D265C)* | TKVDKKVEPKSCDKTHTCPPCPAPEAAGGP |
| | | SVFLFPPKPKDTLMISRTPEVTCVVVCVSHE |
| | | DPEVKFNWYVDGVEVHNAKTKPREEQYNS |
| | | TYRVVSVLTVLHQDWLNGKEYKCKVSNKAL |
| | | PAPIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNAYTQKSLSLSPGK |
| 107 | Human CD45RA | MTMYLWLKLLAFGFAFLDTEVFVTGQSPTPSPTG |
| | Isoform (Uniprot | LTTAKMPSVPLSSDPLPTHTTAFSPASTFEREND |
| | Accession No: | FSETTTSLSPDNTSTQVSPDSLDNASAFNTTDAY |
| | P08575-8) | LNASETTTLSPSGSAVISTTTIATTPSKPTCDEKY |
| | | ANITVDYLYNKETKLFTAKLNVNENVECGNNTCT |
| | | NNEVHNLTECKNASVSISHNSCTAPDKTLILDVPP |
| | | GVEKFQLHDCTQVEKADTTICLKWKNIETFTCDT |
| | | QNITYRFQCGNMIFDNKEIKLENLEPEHEYKCDS |
| | | EILYNNHKFTNASKIIKTDFGSPGEPQIIFCRSEAA |
| | | HQGVITWNPPQRSFHNFTLCYIKETEKDCLNLDK |
| | | NLIKYDLQNLKPYTKYVLSLHAYIIAKVQRNGSAA |
| | | MCHFTTKSAPPSQVWNMTVSMTSDNSMHVKCR |
| | | PPRDRNGPHERYHLEVEAGNTLVRNESHKNCDF |
| | | RVKDLQYSTDYTFKAYFHNGDYPGEPFILHHSTS |
| | | YNSKALIAFLAFLIIVTSIALLVVLYKIYDLHKKRSC |
| | | NLDEQQELVERDDEKQLMNVEPIHADILLETYKR |
| | | KIADEGRLFLAEFQSIPRVFSKFPIKEARKPFNQN |
| | | KNRYVDILPYDYNRVELSEINGDAGSNYINASYID |
| | | GFKEPRKYIAAQGPRDETVDDFWRMIWEQKATV |
| | | IVMVTRCEEGNRNKCAEYWPSMEEGTRAFGDV |
| | | VVKINQHKRCPDYIIQKLNIVNKKEKATGREVTHI |
| | | QFTSWPDHGVPEDPHLLLKLRRRVNAFSNFFSG |
| | | PIVVHCSAGVGRTGTYIGIDAMLEGLEAENKVDV |
| | | YGYVVKLRRQRCLMVQVEAQYILIHQALVEYNQF |
| | | GETEVNLSELHPYLHNMKKRDPPSEPSPLEAEF |
| | | QRLPSYRSWRTQHIGNQEENKSKNRNSNVIPYD |
| | | YNRVPLKHELEMSKESEHDSDESSDDDSDSEEP |
| | | SKYINASFIMSYWKPEVMIAAQGPLKETIGDFWQ |
| | | MIFQRKVKVIVMLTELKHGDQEICAQYWGEGKQT |
| | | YGDIEVDLKDTDKSSTYTLRVFELRHSKRKDSRT |
| | | VYQYQYTNWSVEQLPAEPKELISMIQVVKQKLPQ |
| | | KNSSEGNKHHKSTPLLIHCRDGSQQTGIFCALLN |
| | | LLESAETEEVVDIFQVVKALRKARPGMVSTFEQY |
| | | QFLYDVIASTYPAQNGQVKKNNHQEDKIEFDNEV |
| | | DKVKQDANCVNPLGAPEKLPEAKEQAEGSEPTS |
| | | GTEGPEHSVNGPASPALNQGS |
| 108 | Human CD45RO | MTMYLWLKLLAFGFAFLDTEVFVTGQSPTPSPTD |
| | Isoform (NCBI | AYLNASETTTLSPSGSAVISTTTIATTPSKPTCDE |
| | Accession No: | KYANITVDYLYNKETKLFTAKLNVNENVECGNNT |
| | NP_563578.2) | CTNNEVHNLTECKNASVSISHNSCTAPDKTLILDV |
| | | PPGVEKFQLHDCTQVEKADTTICLKWKNIETFTC |
| | | DTQNITYRFQCGNMIFDNKEIKLENLEPEHEYKC |
| | | DSEILYNNHKFTNASKIIKTDFGSPGEPQIIFCRSE |
| | | AAHQGVITWNPPQRSFHNFTLCYIKETEKDCLNL |
| | | DKNLIKYDLQNLKPYTKYVLSLHAYIIAKVQRNGS |
| | | AAMCHFTTKSAPPSQVWNMTVSMTSDNSMHVK |
| | | CRPPRDRNGPHERYHLEVEAGNTLVRNESHKN |
| | | CDFRVKDLQYSTDYTFKAYFHNGDYPGEPFILHH |
| | | STSYNSKALIAFLAFLIIVTSIALLVVLYKIYDLHKKR |
| | | SCNLDEQQELVERDDEKQLMNVEPIHADILLETY |
| | | KRKIADEGRLFLAEFQSIPRVFSKFPIKEARKPFN |
| | | QNKNRYVDILPYDYNRVELSEINGDAGSNYINAS |
| | | YIDGFKEPRKYIAAQGPRDETVDDFWRMIWEQK |
| | | ATVIVMVTRCEEGNRNKCAEYWPSMEEGTRAFG |
| | | DVVVKINQHKRCPDYIIQKLNIVNKKEKATGREVT |
| | | HIQFTSWPDHGVPEDPHLLLKLRRRVNAFSNFFS |
| | | GPIVVHCSAGVGRTGTYIGIDAMLEGLEAENKVD |
| | | VYGYVVKLRRQRCLMVQVEAQYILIHQALVEYNQ |
| | | FGETEVNLSELHPYLHNMKKRDPPSEPSPLEAEF |
| | | QRLPSYRSWRTQHIGNQEENKSKNRNSNVIPYD |
| | | YNRVPLKHELEMSKESEHDSDESSDDDSDSEEP |
| | | SKYINASFIMSYWKPEVMIAAQGPLKETIGDFWQ |
| | | MIFQRKVKVIVMLTELKHGDQEICAQYWGEGKQT |
| | | YGDIEVDLKDTDKSSTYTLRVFELRHSKRKDSRT |
| | | VYQYQYTNWSVEQLPAEPKELISMIQVVKQKLPQ |
| | | KNSSEGNKHHKSTPLLIHCRDGSQQTGIFCALLN |
| | | LLESAETEEVVDIFQVVKALRKARPGMVSTFEQY |
| | | QFLYDVIASTYPAQNGQVKKNNHQEDKIEFDNEV |
| | | DKVKQDANCVNPLGAPEKLPEAKEQAEGSEPTS |
| | | GTEGPEHSVNGPASPALNQGS |
| 109 | Human CD45RB | MTMYLWLKLLAFGFAFLDTEVFVTGQSPTPSPTG |
| | Isoform (NCBI | VSSVQTPHLPTHADSQTPSAGTDTQTFSGSAAN |
| | Accession No: | AKLNPTPGSNAISDAYLNASETTTLSPSGSAVIST |
| | XP_006711537.1) | TTIATTPSKPTCDEKYANITVDYLYNKETKLFTAKL |
| | | NVNENVECGNNTCTNNEVHNLTECKNASVSISH |
| | | NSCTAPDKTLILDVPPGVEKFQLHDCTQVEKADT |
| | | TICLKWKNIETFTCDTQNITYRFQCGNMIFDNKEI |
| | | KLENLEPEHEYKCDSEILYNNHKFTNASKIIKTDF |
| | | GSPGEPQIIFCRSEAAHQGVITWNPPQRSFHNFT |
| | | LCYIKETEKDCLNLDKNLIKYDLQNLKPYTKYVLS |
| | | LHAYIIAKVQRNGSAAMCHFTTKSAPPSQVWNM |
| | | TVSMTSDNSMHVKCRPPRDRNGPHERYHLEVE |
| | | AGNTLVRNESHKNCDFRVKDLQYSTDYTFKAYF |
| | | HNGDYPGEPFILHHSTSYNSKALIAFLAFLIIVTSIA |
| | | LLVVLYKIYDLHKKRSCNLDEQQELVERDDEKQL |
| | | MNVEPIHADILLETYKRKIADEGRLFLAEFQSIPRV |
| | | FSKFPIKEARKPFNQNKNRYVDILPYDYNRVELS |
| | | EINGDAGSNYINASYIDGFKEPRKYIAAQGPRDET |
| | | VDDFWRMIWEQKATVIVMVTRCEEGNRNKCAEY |
| | | WPSMEEGTRAFGDVVVKINQHKRCPDYIIQKLNI |
| | | VNKKEKATGREVTHIQFTSWPDHGVPEDPHLLLK |
| | | LRRRVNAFSNFFSGPIVVHCSAGVGRTGTYIGID |
| | | AMLEGLEAENKVDVYGYVVKLRRQRCLMVQVEA |
| | | QYILIHQALVEYNQFGETEVNLSELHPYLHNMKK |
| | | RDPPSEPSPLEAEFQRLPSYRSWRTQHIGNQEE |
| | | NKSKNRNSNVIPYDYNRVPLKHELEMSKESEHD |
| | | SDESSDDDSDSEEPSKYINASFIMSYWKPEVMIA |
| | | AQGPLKETIGDFWQMIFQRKVKVIVMLTELKHGD |
| | | QEICAQYWGEGKQTYGDIEVDLKDTDKSSTYTLR |
| | | VFELRHSKRKDSRTVYQYQYTNWSVEQLPAEPK |
| | | ELISMIQVVKQKLPQKNSSEGNKHHKSTPLLIHCR |
| | | DGSQQTGIFCALLNLLESAETEEVVDIFQVVKALR |
| | | KARPGMVSTFEQYQFLYDVIASTYPAQNGQVKK |
| | | NNHQEDKIEFDNEVDKVKQDANCVNPLGAPEKL |
| | | PEAKEQAEGSEPTSGTEGPEHSVNGPASPALNQ |
| | | GS |
| 110 | Human CD45RAB | MTMYLWLKLLAFGFAFLDTEVFVTGQSPTPSPTG |
| | Isoform (NCBI | LTTAKMPSVPLSSDPLPTHTTAFSPASTFEREND |
| | Accession No: | FSETTTSLSPDNTSTQVSPDSLDNASAFNTTGVS |
| | XP_006711535.1) | SVQTPHLPTHADSQTPSAGTDTQTFSGSAANAK |
| | | LNPTPGSNAISDAYLNASETTTLSPSGSAVISTTTI |
| | | ATTPSKPTCDEKYANITVDYLYNKETKLFTAKLNV |
| | | NENVECGNNTCTNNEVHNLTECKNASVSISHNS |
| | | CTAPDKTLILDVPPGVEKFQLHDCTQVEKADTTIC |
| | | LKWKNIETFTCDTQNITYRFQCGNMIFDNKEIKLE |
| | | NLEPEHEYKCDSEILYNNHKFTNASKIIKTDFGSP |
| | | GEPQIIFCRSEAAHQGVITWNPPQRSFHNFTLCYI |
| | | KETEKDCLNLDKNLIKYDLQNLKPYTKYVLSLHAY |
| | | IIAKVQRNGSAAMCHFTTKSAPPSQVWNMTVSM |
| | | TSDNSMHVKCRPPRDRNGPHERYHLEVEAGNT |
| | | LVRNESHKNCDFRVKDLQYSTDYTFKAYFHNGD |
| | | YPGEPFILHHSTSYNSKALIAFLAFLIIVTSIALLVVL |
| | | YKIYDLHKKRSCNLDEQQELVERDDEKQLMNVE |
| | | PIHADILLETYKRKIADEGRLFLAEFQSIPRVFSKF |
| | | PIKEARKPFNQNKNRYVDILPYDYNRVELSEINGD |
| | | AGSNYINASYIDGFKEPRKYIAAQGPRDETVDDF |
| | | WRMIWEQKATVIVMVTRCEEGNRNKCAEYWPS |
| | | MEEGTRAFGDVVVKINQHKRCPDYIIQKLNIVNKK |
| | | EKATGREVTHIQFTSWPDHGVPEDPHLLLKLRRR |
| | | VNAFSNFFSGPIVVHCSAGVGRTGTYIGIDAMLE |
| | | GLEAENKVDVYGYVVKLRRQRCLMVQVEAQYILI |
| | | HQALVEYNQFGETEVNLSELHPYLHNMKKRDPP |
| | | SEPSPLEAEFQRLPSYRSWRTQHIGNQEENKSK |
| | | NRNSNVIPYDYNRVPLKHELEMSKESEHDSDES |
| | | SDDDSDSEEPSKYINASFIMSYWKPEVMIAAQGP |
| | | LKETIGDFWQMIFQRKVKVIVMLTELKHGDQEICA |
| | | QYWGEGKQTYGDIEVDLKDTDKSSTYTLRVFEL |
| | | RHSKRKDSRTVYQYQYTNWSVEQLPAEPKELIS |
| | | MIQVVKQKLPQKNSSEGNKHHKSTPLLIHCRDGS |
| | | QQTGIFCALLNLLESAETEEVVDIFQVVKALRKAR |
| | | PGMVSTFEQYQFLYDVIASTYPAQNGQVKKNNH |
| | | QEDKIEFDNEVDKVKQDANCVNPLGAPEKLPEA |
| | | KEQAEGSEPTSGTEGPEHSVNGPASPALNQGS |
| 111 | Human CD45RBC | MTMYLWLKLLAFGFAFLDTEVFVTGQSPTPSPTG |
| | Isoform (NCBI | VSSVQTPHLPTHADSQTPSAGTDTQTFSGSAAN |
| | Accession No: | AKLNPTPGSNAISDVPGERSTASTFPTDPVSPLT |
| | XP_006711536.1) | TTLSLAHHSSAALPARTSNTTITANTSDAYLNASE |
| | | TTTLSPSGSAVISTTTIATTPSKPTCDEKYANITVD |
| | | YLYNKETKLFTAKLNVNENVECGNNTCTNNEVH |
| | | NLTECKNASVSISHNSCTAPDKTLILDVPPGVEKF |
| | | QLHDCTQVEKADTTICLKWKNIETFTCDTQNITYR |
| | | FQCGNMIFDNKEIKLENLEPEHEYKCDSEILYNNH |
| | | KFTNASKIIKTDFGSPGEPQIIFCRSEAAHQGVIT |
| | | WNPPQRSFHNFTLCYIKETEKDCLNLDKNLIKYD |
| | | LQNLKPYTKYVLSLHAYIIAKVQRNGSAAMCHFTT |
| | | KSAPPSQVWNMTVSMTSDNSMHVKCRPPRDRN |
| | | GPHERYHLEVEAGNTLVRNESHKNCDFRVKDLQ |
| | | YSTDYTFKAYFHNGDYPGEPFILHHSTSYNSKALI |
| | | AFLAFLIIVTSIALLVVLYKIYDLHKKRSCNLDEQQE |
| | | LVERDDEKQLMNVEPIHADILLETYKRKIADEGRL |
| | | FLAEFQSIPRVFSKFPIKEARKPFNQNKNRYVDIL |
| | | PYDYNRVELSEINGDAGSNYINASYIDGFKEPRK |
| | | YIAAQGPRDETVDDFWRMIWEQKATVIVMVTRC |
| | | EEGNRNKCAEYWPSMEEGTRAFGDVVVKINQH |
| | | KRCPDYIIQKLNIVNKKEKATGREVTHIQFTSWPD |
| | | HGVPEDPHLLLKLRRRVNAFSNFFSGPIVVHCSA |
| | | GVGRTGTYIGIDAMLEGLEAENKVDVYGYVVKLR |
| | | RQRCLMVQVEAQYILIHQALVEYNQFGETEVNLS |
| | | ELHPYLHNMKKRDPPSEPSPLEAEFQRLPSYRS |
| | | WRTQHIGNQEENKSKNRNSNVIPYDYNRVPLKH |
| | | ELEMSKESEHDSDESSDDDSDSEEPSKYINASFI |
| | | MSYWKPEVMIAAQGPLKETIGDFWQMIFQRKVK |
| | | VIVMLTELKHGDQEICAQYWGEGKQTYGDIEVDL |
| | | KDTDKSSTYTLRVFELRHSKRKDSRTVYQYQYT |
| | | NWSVEQLPAEPKELISMIQVVKQKLPQKNSSEGN |
| | | KHHKSTPLLIHCRDGSQQTGIFCALLNLLESAETE |
| | | EVVDIFQVVKALRKARPGMVSTFEQYQFLYDVIA |
| | | STYPAQNGQVKKNNHQEDKIEFDNEVDKVKQDA |
| | | NCVNPLGAPEKLPEAKEQAEGSEPTSGTEGPEH |
| | | SVNGPASPALNQGS |
| 112 | Human CD45RABC | MTMYLWLKLLAFGFAFLDTEVFVTGQSPTPSPTG |
| | Isoform (NCBI | LTTAKMPSVPLSSDPLPTHTTAFSPASTFEREND |
| | Accession No. | FSETTTSLSPDNTSTQVSPDSLDNASAFNTTGVS |
| | NP_002829.3) | SVQTPHLPTHADSQTPSAGTDTQTFSGSAANAK |
| | | LNPTPGSNAISDVPGERSTASTFPTDPVSPLTTTL |
| | | SLAHHSSAALPARTSNTTITANTSDAYLNASETTT |
| | | LSPSGSAVISTTTIATTPSKPTCDEKYANITVDYLY |
| | | NKETKLFTAKLNVNENVECGNNTCTNNEVHNLT |
| | | ECKNASVSISHNSCTAPDKTLILDVPPGVEKFQLH |
| | | DCTQVEKADTTICLKWKNIETFTCDTQNITYRFQC |
| | | GNMIFDNKEIKLENLEPEHEYKCDSEILYNNHKFT |
| | | NASKIIKTDFGSPGEPQIIFCRSEAAHQGVITWNP |
| | | PQRSFHNFTLCYIKETEKDCLNLDKNLIKYDLQNL |
| | | KPYTKYVLSLHAYIIAKVQRNGSAAMCHFTTKSA |
| | | PPSQVWNMTVSMTSDNSMHVKCRPPRDRNGP |
| | | HERYHLEVEAGNTLVRNESHKNCDFRVKDLQYS |
| | | TDYTFKAYFHNGDYPGEPFILHHSTSYNSKALIAF |
| | | LAFLIIVTSIALLVVLYKIYDLHKKRSCNLDEQQELV |
| | | ERDDEKQLMNVEPIHADILLETYKRKIADEGRLFL |
| | | AEFQSIPRVFSKFPIKEARKPFNQNKNRYVDILPY |
| | | DYNRVELSEINGDAGSNYINASYIDGFKEPRKYIA |
| | | AQGPRDETVDDFWRMIWEQKATVIVMVTRCEEG |
| | | NRNKCAEYWPSMEEGTRAFGDVVVKINQHKRC |
| | | PDYIIQKLNIVNKKEKATGREVTHIQFTSWPDHGV |
| | | PEDPHLLLKLRRRVNAFSNFFSGPIVVHCSAGVG |
| | | RTGTYIGIDAMLEGLEAENKVDVYGYVVKLRRQR |
| | | CLMVQVEAQYILIHQALVEYNQFGETEVNLSELH |
| | | PYLHNMKKRDPPSEPSPLEAEFQRLPSYRSWRT |
| | | QHIGNQEENKSKNRNSNVIPYDYNRVPLKHELE |
| | | MSKESEHDSDESSDDDSDSEEPSKYINASFIMSY |
| | | WKPEVMIAAQGPLKETIGDFWQMIFQRKVKVIVM |
| | | LTELKHGDQEICAQYWGEGKQTYGDIEVDLKDT |
| | | DKSSTYTLRVFELRHSKRKDSRTVYQYQYTNWS |
| | | VEQLPAEPKELISMIQVVKQKLPQKNSSEGNKHH |
| | | KSTPLLIHCRDGSQQTGIFCALLNLLESAETEEVV |
| | | DIFQVVKALRKARPGMVSTFEQYQFLYDVIASTY |
| | | PAQNGQVKKNNHQEDKIEFDNEVDKVKQDANCV |
| | | NPLGAPEKLPEAKEQAEGSEPTSGTEGPEHSVN |
| | | GPASPALNQGS |
| 113 | Human CD45RABC | QSPTPSPTGLTTAKMPSVPLSSDPLPTHTTAFSP |
| | Antigen (Fragment of | ASTFERENDFSETTTSLSPDNTSTQVSPDSLDNA |
| | Human CD45RABC | SAFNTTGVSSVQTPHLPTHADSQTPSAGTDTQT |
| | Isoform) | FSGSAANAKLNPTPGSNAISDVPGERSTASTFPT |
| | | DPVSPLTTTLSLAHHSSAALPARTSNTTITANTSD |
| | | AYLNASETTTLSPSGSAVISTTTIATTPSKPTCDE |
| | | KYANITVDYLYNKETKLFTAKLNVNENVECGNNT |
| | | CTNNEVHNLTECKNASVSISHNSCTAPDKTLILDV |
| | | PPGVEKFQLHDCTQVEKADTTICLKWKNIETFTC |
| | | DTQNITYRFQCGNMIFDNKEIKLENLEPEHEYKC |
| | | DSEILYNNHKFTNASKIIKTDFGSPGEPQIIFCRSE |
| | | AAHQGVITWNPPQRSFHNFTLCYIKETEKDCLNL |
| | | DKNLIKYDLQNLKPYTKYVLSLHAYIIAKVQRNGS |
| | | AAMCHFTTKSAPPSQVWNMTVSMTSDNSMHVK |
| | | CRPPRDRNGPHERYHLEVEAGNTLVRNESHKN |
| | | CDFRVKDLQYSTDYTFKAYFHNGDYPGEPFILHH |
| | | STSYNSK |
| 114-132 | Sequences described in sequence listing | |
| 133 | Ab5 Heavy Chain | GAAGTGCAGCTTGTGGAGTCCGGTGGCGGAC |
| | Variable Region | TGGTCCAGCCGGGCGGATCTCTGAGACTTTCG |
| | (Nucleic Acid) | TGTGCCGCCTCGGGATTCACCTTCGAAGCGTA |
| | | TTCCATGAACTGGGTCAGACAGGCCCCCGGAA |
| | | AGGGCCTGGAATGGGTGTCGTACATTAGCCTG |
| | | TCGGGGGCCACCATCCATTACGCCGATAGCGT |
| | | GAAGGGCCGGTTCACAATCTCCCGGGACAAC |
| | | GCCAAGAACTCCCTCTACCTCCAAATGAACAG |
| | | CCTGCGCGCTGAGGACACTGCTGTGTACTATT |
| | | GCGCGAGGGGTGGCCAGTACTACTACACCTCA |
| | | AGCGACTACGGCGAAGTGGCATTCGATATCTG |
| | | GGGACAGGGGACCATGGTCACCGTCAGCTCC |
| 134 | Ab5 Light Chain | GATATCGTGATGACACAGTCCCCTCTGTCCCT |
| | Variable Region | CCCTGTGACCCCCGGAGAACCAGCCTCTATTT |
| | (Nucleic Acid) | CCTGCCGGTCCTCCCAATCCCTGGTGTCCTCG |
| | | GGTTATAACTACCTGGATTGGTACTTGCAAAAG |
| | | CCCGGACAGAGCCCCCAGCTGCTCATCTACTT |
| | | CGGAAGCTCACGCGCGAGCGGGGTGCCGGAT |
| | | AGGTTTTCGGGATCCGGAAGCGGCACCGACTT |
| | | CACGCTGAAGATCTCGAGAGTCGAGGCCGAG |
| | | GACGTGGGCGTGTACTACTGTATGCAGCGGC |
| | | GGCGCACCCCCTGGTCCTTCGGGGGGGGAAC |
| | | TAAGGTCGAGATCAAG |
| 135-161 | Sequences described in sequence listing | |
| 162 | Ab5 Heavy Chain | ggtaccgccaccatggatctgctgcacaagaacatgaagcacctg |
| | DNA nucleotide | tggttctttctgctgctggtggccgctcctagatgggtgctgtctgaagtg |
| | sequence (coding | cagctggtggaatctggcggaggattggttcagcctggcggctctctg |
| | region underlined; | agactgtcttgtgctgcctctggcttcaccttcgaggcctactccatgaa |
| | restriction site in | ctgggtccgacaggctcctggcaaaggactggaatgggtgtcctac |
| | bold; Kozak sequence | atctctctgtctggcgccaccatccactacgccgattctgtgaagggc |
| | in italics) | agattcaccatcagccgggacaacgccaagaactccctgtacctgc |
| | | agatgaacagcctgagagccgaggacaccgccgtgtactattgtgc |
| | | tagaggcggccagtactactacacctcctctgattacggcgaggtgg |
| | | ccttcgatatctggggccagggaacaatggtcaccgtgtcctctgctt |
| | | ccaccaagggaccttccgtgtttcctctggctccttccagcaagtctac |
| | | ctctggtggaaccgctgctctgggctgcctggtcaaggattactttcct |
| | | gagcctgtgaccgtgtcttggaactctggtgctctgacctccggcgtg |
| | | cacacatttccagctgtgctgcagtcctccggcctgtactctctgtcctc |
| | | tgtcgtgaccgtgccttctagctctctgggcacccagacctacatctgc |
| | | aacgtgaaccacaagccttccaacaccaaggtggacaagaaggt |
| | | ggaacccaagtcctgcgacaagacccacacctgtcctccatgtcct |
| | | gctccagaagctgctggcggaccctctgtgttcctgtttccacctaagc |
| | | ctaaggacaccctgatgatctctcggacccctgaagtgacctgcgtg |
| | | gtcgtgtgtgtgtctcacgaggacccagaagtgaagttcaattggtac |
| | | gtggacggcgtggaagtgcacaatgccaagaccaagcctagaga |
| | | ggaacagtacaactccacctacagagtggtgtccgtgctgaccgtg |
| | | ctgcaccaggattggctgaacggcaaagagtacaagtgcaaggtg |
| | | tccaacaaggccctgcctgctcctatcgaaaagaccatctccaagg |
| | | ctaagggccagcctcgggaacctcaggtttacacactgcctccatct |
| | | cgggacgagctgaccaagaatcaggtgtccctgacctgcctcgtga |
| | | agggcttctacccttccgatatcgccgtggaatgggagtctaacggc |
| | | cagccagagaacaactacaagacaacccctcctgtgctggactcc |
| | | gacggctcattcttcctgtactccaagctgacagtggacaagtctcgg |
| | | tagcagcagggcaacgtgttctcctgttctgtgatgcacgaggccctg |
| | | cacaacgcctacacacagaagtccctgtctctgtcccctggcaagtg |
| | | atagaagctt |
| 163 | Ab5 Light Chain DNA | ggatccgccaccatggatctgctgcacaagaacatgaagcacctg |
| | nucleotide sequence | tagttctttctgctgctggtggccgctcctagatgggtgctgtctgacatc |
| | (coding region | gtgatgacccagtctccactgagcctgcctgttacacctggcgagcct |
| | underlined; restriction | gccagcatctcctgcagatottcccagtctctggtgtcctccggctaca |
| | site in bold; Kozak | actacctggactggtatctgcagaagcccggccagtctcctcagctg |
| | sequence in italics) | ctgatctactttggctcctccagagcctctggcgtgcccgatagattttct |
| | | ggctctagctccggcaccgacttcaccctgaagatctctagagtgga |
| | | agccgaggacgtgggcgtgtactactgtatgcagcggagaagaac |
| | | cccttggtctttcggcggaggcaccaaggtggaaatcaagagaacc |
| | | gtggctgccccttccgtgttcatcttcccaccatctgacgagcagctga |
| | | agtctggcaccgcttctgtcgtgtgcctgctgaacaacttctaccctcg |
| | | ggaagccaaggtgcagtggaaggtggacaatgccctgcagtccg |
| | | gcaactcccaagagtctgtgaccgagcaggactccaaggactcta |
| | | cctacagcctgtcctccacactgaccctgtctaaggccgactacgag |
| | | aagcacaaggtgtacgcctgtgaagtgacccaccagggactgtcta |
| | | gccccgtgaccaagtctttcaacagaggcgagtgctgatagctcga |
| | | g |

### OTHER EMBODIMENTS

All publications, patents, and patent applications mentioned in this specification are incorporated herein by reference to the same extent as if each independent publication or patent application was specifically and individually indicated to be incorporated by reference.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. (canceled)

2. A method of conditioning a human subject in need of a hematopoietic stem cell (HSC) transplant, the method comprising administering to the human subject a single dose of at least about 0.15-0.4 mg/kg of an antibody-drug conjugate (ADC), such that the human subject is conditioned for an HSC transplant, wherein the ADC comprises an indolinobenzodiazepine (IGN) cytotoxin conjugated via a linker to an anti-CD45 antibody comprising a heavy chain variable region comprising a three complimentary determining regions CDR1, CDR2, and CDR3 within the heavy chain variable region, wherein the heavy chain variable region comprises the amino acid sequence of SEQ ID NO:41; and comprising a light chain variable region comprising three complimentary determining regions CDR1, CDR2, and CDR3 within the light chain variable region, wherein the light chain variable region comprises the amino acid sequence of of SEQ ID NO: 45.

3. The method of claim 2, further comprising administering an HSC transplant to the human subject.

4. The method of claim 3, wherein the HSC transplant is administered about 10 days after the single dose of the ADC.

5. The method of claim 2, wherein the human subject has a disorder treatable with a transplant of genetically-modified cells.

6. The method of claim 5, wherein the disorder treatable with the transplant of genetically-modified cells is a hemoglobinopathy disorder.

7.-12. (canceled)

13. The method of claim 2, wherein the ADC is administered to the human subject at a single dose of 0.3 mg/kg.

14.-16. (canceled)

17. The method of claim 2, wherein the anti-CD45 antibody comprises a heavy chain variable region comprising the CDR1, CDR2, and CDR3 sequences, wherein the amino acid sequences of CDR1, CDR2, and CDR3 comprises SEQ ID NOs: 42, 43, and 44, respectively; and a light chain variable region comprising the CDR1, CDR2, and CDR3 sequences, wherein the amino acid sequences of CDR1, CDR2, and CDR3 comprises SEQ ID NOs: 46, 47, and 48, respectively.

18. The method of claim 2, wherein the anti-CD45 antibody is an IgG h isotype.

19. The method of claim 2, wherein the anti-CD45 antibody comprises a heavy chain amino acid sequence as set forth in SEQ ID NO:49, and a light chain amino acid sequence as set forth in SEQ ID NO:50.

20. (canceled)

21. The method of claim 2, wherein the IGN is an IGN dimer or an IGN pseudo dimer.

22. The method of claim 21, wherein the IGN pseudo dimer is represented by: 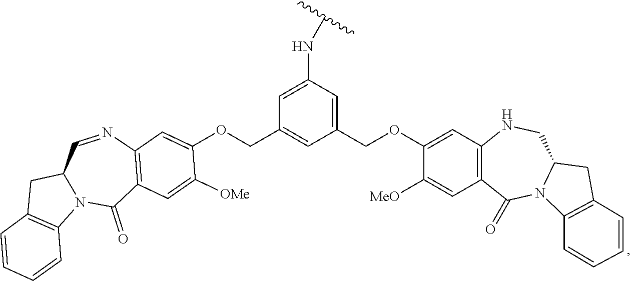 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]
wherein the wavy line indicates the point of covalent attachment to the linker of the ADC, or
wherein the IGN pseudo dimer is represented by: 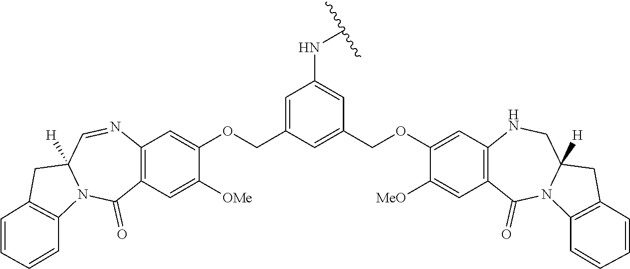 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]
wherein the wavy line indicates the point of covalent attachment to the linker of the ADC.

23-25. (canceled)

26. The method of claim 2, wherein the cytotoxin-linker conjugate, prior to conjugation to the antibody or antigen binding portion thereof, and including a reactive substituent Z′, taken together as Cy-L-Z′, has a structure:

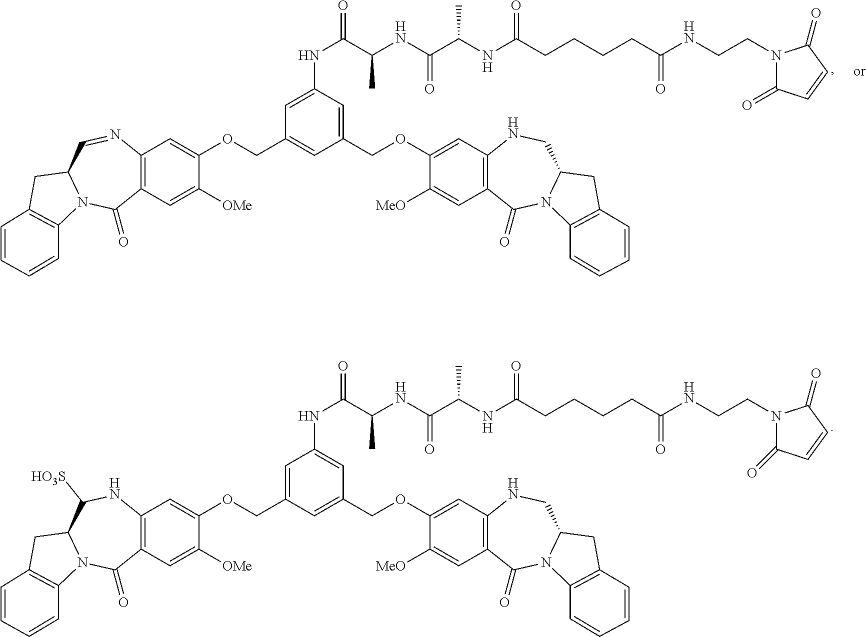

27. The method of claim 2, wherein the ADC has a formula: 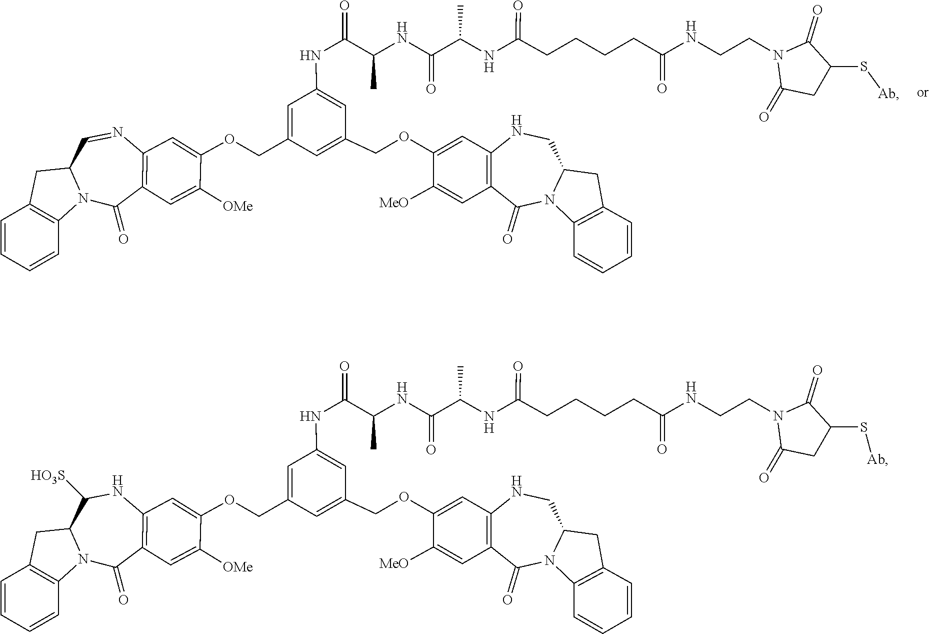
wherein Ab is the anti-CD45 antibody.

28. A method of conditioning a human subject in need thereof, the method comprising administering to the human subject a dose of 0.15 mg/kg to 0.4 mg/kg of an antibody-drug conjugate (ADC) comprising a cytotoxin conjugated to an anti-CD45 antibody (Ab) via a linker, such that the human subject is conditioned for a stem cell transplant, wherein the ADC has a formula 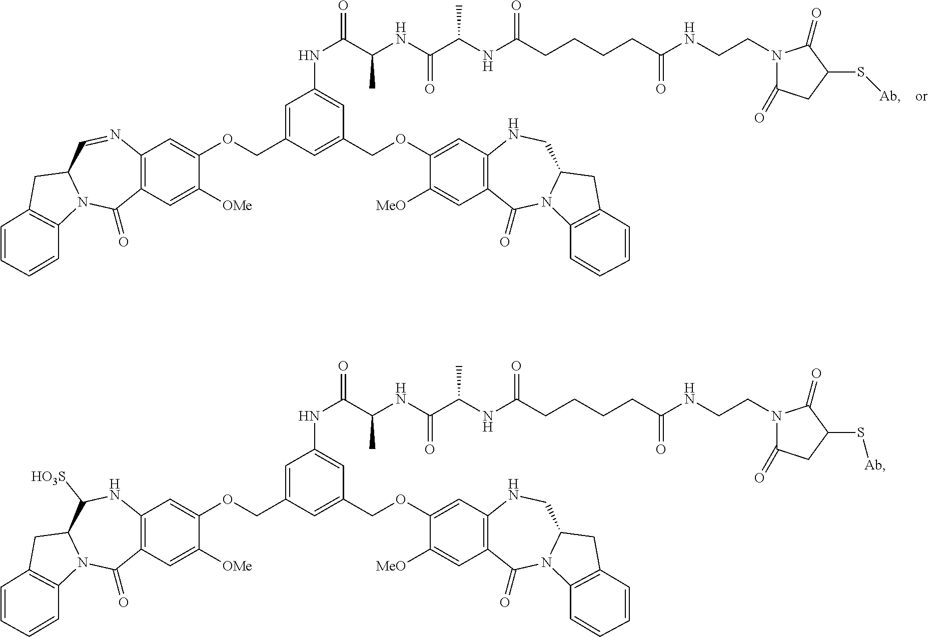
wherein the anti-CD45 antibody comprises a heavy chain variable region comprising three complimentary determining regions CDR1, CDR2, and CDR3 within the heavy chain variable region, wherein the heavy chain variable region comprises the amino acid sequence of SEQ ID NO:41; comprises a light chain variable region comprising a CDR1 domain comprising three complimentary determining regions CDR1, CDR2, and CDR3 within the light chain variable region, wherein the light chain variable region comprises the amino acid sequence of SEQ ID NO: 45; and is an IgG1 isotype.

29. The method of claim 28, wherein the anti-CD45 antibody comprises a heavy chain comprising an amino acid sequence of SEQ ID NO: 49, and a light chain comprising an amino acid sequence of SEQ ID NO: 50.

30.-32. (canceled)

33. The method of claim 28, wherein the ADC is administered to the human subject at a dose of 0.3 mg/kg.

34.-36. (canceled)

37. The method of claim 28, wherein a single dose of the ADC is administered to the human subject.

38. The method of claim 28, wherein the ADC is a monotherapy conditioning agent.

39. The method of claim 28, wherein the human patient has a hematological cancer.

40. (canceled)

41. The method of claim 28, further comprising administering an HSC transplant to the human patient.

42. (canceled)
